SEQUENCE LISTING (1 sequence)

SEQ ID NO: 1 - RNA, 10653 nt, Parabacteroides distasonis
  source 1..10653: mol_type = rRNA
  modified_base 9: mod_base = OTHER; note = thymine
  modified_base 11..12: mod_base = OTHER; note = thymine
  modified_base 24..25: mod_base = OTHER; note = thymine
  modified_base 30: mod_base = OTHER; note = thymine
  modified_base 36: mod_base = OTHER; note = thymine
  modified_base 40..41: mod_base = OTHER; note = thymine
  modified_base 43..44: mod_base = OTHER; note = thymine
  modified_base 50..51: mod_base = OTHER; note = thymine
  modified_base 60: mod_base = OTHER; note = thymine
  modified_base 67..70: mod_base = OTHER; note = thymine
  modified_base 75: mod_base = OTHER; note = thymine
  modified_base 83: mod_base = OTHER; note = thymine
  modified_base 86: mod_base = OTHER; note = thymine
  modified_base 92..93: mod_base = OTHER; note = thymine
  modified_base 100..101: mod_base = OTHER; note = thymine
  modified_base 106: mod_base = OTHER; note = thymine
  modified_base 116: mod_base = OTHER; note = thymine
  modified_base 121: mod_base = OTHER; note = thymine
  modified_base 125..126: mod_base = OTHER; note = thymine
  modified_base 136: mod_base = OTHER; note = thymine
  modified_base 138: mod_base = OTHER; note = thymine
  modified_base 140: mod_base = OTHER; note = thymine
  modified_base 157: mod_base = OTHER; note = thymine
  modified_base 159..160: mod_base = OTHER; note = thymine
  modified_base 171: mod_base = OTHER; note = thymine
  modified_base 175: mod_base = OTHER; note = thymine
  modified_base 178: mod_base = OTHER; note = thymine
  modified_base 185..186: mod_base = OTHER; note = thymine
  modified_base 189: mod_base = OTHER; note = thymine
  modified_base 196: mod_base = OTHER; note = thymine
  modified_base 202..203: mod_base = OTHER; note = thymine
  modified_base 211: mod_base = OTHER; note = thymine
  modified_base 216..217: mod_base = OTHER; note = thymine
  modified_base 224: mod_base = OTHER; note = thymine
  modified_base 229: mod_base = OTHER; note = thymine
  modified_base 236: mod_base = OTHER; note = thymine
  modified_base 243: mod_base = OTHER; note = thymine
  modified_base 246..248: mod_base = OTHER; note = thymine
  modified_base 254..255: mod_base = OTHER; note = thymine
  modified_base 259: mod_base = OTHER; note = thymine
  modified_base 263: mod_base = OTHER; note = thymine
  modified_base 265: mod_base = OTHER; note = thymine
  modified_base 273: mod_base = OTHER; note = thymine
  modified_base 278: mod_base = OTHER; note = thymine
  modified_base 282..284: mod_base = OTHER; note = thymine
  modified_base 286: mod_base = OTHER; note = thymine
  modified_base 295..296: mod_base = OTHER; note = thymine
  modified_base 298: mod_base = OTHER; note = thymine
  modified_base 305: mod_base = OTHER; note = thymine
  modified_base 307: mod_base = OTHER; note = thymine
  modified_base 309: mod_base = OTHER; note = thymine
  modified_base 319: mod_base = OTHER; note = thymine
  modified_base 321: mod_base = OTHER; note = thymine
  modified_base 330: mod_base = OTHER; note = thymine
  modified_base 333: mod_base = OTHER; note = thymine
  modified_base 336..338: mod_base = OTHER; note = thymine
  modified_base 343: mod_base = OTHER; note = thymine
  modified_base 346: mod_base = OTHER; note = thymine
  modified_base 354..355: mod_base = OTHER; note = thymine
  modified_base 358: mod_base = OTHER; note = thymine
  modified_base 365..366: mod_base = OTHER; note = thymine
  modified_base 371: mod_base = OTHER; note = thymine
  modified_base 377: mod_base = OTHER; note = thymine
  modified_base 388: mod_base = OTHER; note = thymine
  modified_base 391: mod_base = OTHER; note = thymine
  modified_base 396..398: mod_base = OTHER; note = thymine
  modified_base 402: mod_base = OTHER; note = thymine
  modified_base 404..405: mod_base = OTHER; note = thymine
  modified_base 408: mod_base = OTHER; note = thymine
  modified_base 412: mod_base = OTHER; note = thymine
  modified_base 415: mod_base = OTHER; note = thymine
  modified_base 424..425: mod_base = OTHER; note = thymine
  modified_base 430: mod_base = OTHER; note = thymine
  modified_base 433..434: mod_base = OTHER; note = thymine
  modified_base 436: mod_base = OTHER; note = thymine
  modified_base 442..443: mod_base = OTHER; note = thymine
  modified_base 455: mod_base = OTHER; note = thymine
  modified_base 468: mod_base = OTHER; note = thymine
  modified_base 480: mod_base = OTHER; note = thymine
  modified_base 489: mod_base = OTHER; note = thymine
  modified_base 500: mod_base = OTHER; note = thymine
  modified_base 502..503: mod_base = OTHER; note = thymine
  modified_base 506: mod_base = OTHER; note = thymine
  modified_base 515: mod_base = OTHER; note = thymine
  modified_base 517..519: mod_base = OTHER; note = thymine
  modified_base 525: mod_base = OTHER; note = thymine
  modified_base 529: mod_base = OTHER; note = thymine
  modified_base 535: mod_base = OTHER; note = thymine
  modified_base 539..540: mod_base = OTHER; note = thymine
  modified_base 551: mod_base = OTHER; note = thymine
  modified_base 556..557: mod_base = OTHER; note = thymine
  modified_base 560: mod_base = OTHER; note = thymine
  modified_base 565: mod_base = OTHER; note = thymine
  modified_base 567: mod_base = OTHER; note = thymine
  modified_base 570: mod_base = OTHER; note = thymine
  modified_base 575..576: mod_base = OTHER; note = thymine
  modified_base 585: mod_base = OTHER; note = thymine
  modified_base 587..588: mod_base = OTHER; note = thymine
  modified_base 591: mod_base = OTHER; note = thymine
  modified_base 596..597: mod_base = OTHER; note = thymine
  modified_base 599: mod_base = OTHER; note = thymine
  modified_base 611: mod_base = OTHER; note = thymine
  modified_base 615..616: mod_base = OTHER; note = thymine
  modified_base 619..621: mod_base = OTHER; note = thymine
  modified_base 625..627: mod_base = OTHER; note = thymine
  modified_base 633..634: mod_base = OTHER; note = thymine
  modified_base 642: mod_base = OTHER; note = thymine
  modified_base 645: mod_base = OTHER; note = thymine
  modified_base 653: mod_base = OTHER; note = thymine
  modified_base 657: mod_base = OTHER; note = thymine
  modified_base 661..662: mod_base = OTHER; note = thymine
  modified_base 668..670: mod_base = OTHER; note = thymine
  modified_base 674: mod_base = OTHER; note = thymine
  modified_base 676: mod_base = OTHER; note = thymine
  modified_base 682..683: mod_base = OTHER; note = thymine
  modified_base 688: mod_base = OTHER; note = thymine
  modified_base 690: mod_base = OTHER; note = thymine
  modified_base 692: mod_base = OTHER; note = thymine
  modified_base 703: mod_base = OTHER; note = thymine
  modified_base 706: mod_base = OTHER; note = thymine
  modified_base 709: mod_base = OTHER; note = thymine
  modified_base 712: mod_base = OTHER; note = thymine
  modified_base 715..717: mod_base = OTHER; note = thymine
  modified_base 720: mod_base = OTHER; note = thymine
  modified_base 724: mod_base = OTHER; note = thymine
  modified_base 729: mod_base = OTHER; note = thymine
  modified_base 737: mod_base = OTHER; note = thymine
  modified_base 739: mod_base = OTHER; note = thymine
  modified_base 741: mod_base = OTHER; note = thymine
  modified_base 744: mod_base = OTHER; note = thymine
  modified_base 747: mod_base = OTHER; note = thymine
  modified_base 749..751: mod_base = OTHER; note = thymine
  modified_base 754: mod_base = OTHER; note = thymine
  modified_base 763..765: mod_base = OTHER; note = thymine
  modified_base 768: mod_base = OTHER; note = thymine
  modified_base 772: mod_base = OTHER; note = thymine
  modified_base 778: mod_base = OTHER; note = thymine
  modified_base 782: mod_base = OTHER; note = thymine
  modified_base 785: mod_base = OTHER; note = thymine
  modified_base 789..790: mod_base = OTHER; note = thymine
  modified_base 798: mod_base = OTHER; note = thymine
  modified_base 802..803: mod_base = OTHER; note = thymine
  modified_base 809: mod_base = OTHER; note = thymine
  modified_base 815..816: mod_base = OTHER; note = thymine
  modified_base 818: mod_base = OTHER; note = thymine
  modified_base 822: mod_base = OTHER; note = thymine
  modified_base 825: mod_base = OTHER; note = thymine
  modified_base 827: mod_base = OTHER; note = thymine
  modified_base 829: mod_base = OTHER; note = thymine
  modified_base 831: mod_base = OTHER; note = thymine
  modified_base 835..837: mod_base = OTHER; note = thymine
  modified_base 844: mod_base = OTHER; note = thymine
  modified_base 855..856: mod_base = OTHER; note = thymine
  modified_base 862: mod_base = OTHER; note = thymine
  modified_base 866: mod_base = OTHER; note = thymine
  modified_base 873: mod_base = OTHER; note = thymine
  modified_base 876: mod_base = OTHER; note = thymine
  modified_base 889..891: mod_base = OTHER; note = thymine
  modified_base 901..902: mod_base = OTHER; note = thymine
  modified_base 904: mod_base = OTHER; note = thymine
  modified_base 906: mod_base = OTHER; note = thymine
  modified_base 909..910: mod_base = OTHER; note = thymine
  modified_base 922..924: mod_base = OTHER; note = thymine
  modified_base 931: mod_base = OTHER; note = thymine
  modified_base 935..936: mod_base = OTHER; note = thymine
  modified_base 948: mod_base = OTHER; note = thymine
  modified_base 957..959: mod_base = OTHER; note = thymine
  modified_base 968: mod_base = OTHER; note = thymine
  modified_base 972: mod_base = OTHER; note = thymine
  modified_base 978: mod_base = OTHER; note = thymine
  modified_base 984: mod_base = OTHER; note = thymine
  modified_base 989: mod_base = OTHER; note = thymine
  modified_base 992: mod_base = OTHER; note = thymine
  modified_base 996: mod_base = OTHER; note = thymine
  modified_base 998..999: mod_base = OTHER; note = thymine
  modified_base 1008: mod_base = OTHER; note = thymine
  modified_base 1011: mod_base = OTHER; note = thymine
  modified_base 1021..1022: mod_base = OTHER; note = thymine
  modified_base 1029: mod_base = OTHER; note = thymine
  modified_base 1032..1033: mod_base = OTHER; note = thymine
  modified_base 1035..1036: mod_base = OTHER; note = thymine
  modified_base 1039: mod_base = OTHER; note = thymine
  modified_base 1044: mod_base = OTHER; note = thymine
  modified_base 1048: mod_base = OTHER; note = thymine
  modified_base 1064: mod_base = OTHER; note = thymine
  modified_base 1072..1074: mod_base = OTHER; note = thymine
  modified_base 1076..1077: mod_base = OTHER; note = thymine
  modified_base 1081..1082: mod_base = OTHER; note = thymine
  modified_base 1084: mod_base = OTHER; note = thymine
  modified_base 1093..1095: mod_base = OTHER; note = thymine
  modified_base 1100: mod_base = OTHER; note = thymine
  modified_base 1104: mod_base = OTHER; note = thymine
  modified_base 1111..1112: mod_base = OTHER; note = thymine
  modified_base 1115: mod_base = OTHER; note = thymine
  modified_base 1119: mod_base = OTHER; note = thymine
  modified_base 1128..1129: mod_base = OTHER; note = thymine
  modified_base 1133: mod_base = OTHER; note = thymine
  modified_base 1136..1137: mod_base = OTHER; note = thymine
  modified_base 1143..1144: mod_base = OTHER; note = thymine
  modified_base 1152..1153: mod_base = OTHER; note = thymine
  modified_base 1160: mod_base = OTHER; note = thymine
  modified_base 1162..1163: mod_base = OTHER; note = thymine
  modified_base 1166: mod_base = OTHER; note = thymine
  modified_base 1170: mod_base = OTHER; note = thymine
  modified_base 1173: mod_base = OTHER; note = thymine
  modified_base 1177: mod_base = OTHER; note = thymine
  modified_base 1182: mod_base = OTHER; note = thymine
  modified_base 1188..1190: mod_base = OTHER; note = thymine
  modified_base 1193: mod_base = OTHER; note = thymine
  modified_base 1197: mod_base = OTHER; note = thymine
  modified_base 1199: mod_base = OTHER; note = thymine
  modified_base 1201: mod_base = OTHER; note = thymine
  modified_base 1205: mod_base = OTHER; note = thymine
  modified_base 1211: mod_base = OTHER; note = thymine
  modified_base 1213: mod_base = OTHER; note = thymine
  modified_base 1222..1223: mod_base = OTHER; note = thymine
  modified_base 1226: mod_base = OTHER; note = thymine
  modified_base 1228: mod_base = OTHER; note = thymine
  modified_base 1238: mod_base = OTHER; note = thymine
  modified_base 1240: mod_base = OTHER; note = thymine
  modified_base 1244: mod_base = OTHER; note = thymine
  modified_base 1247: mod_base = OTHER; note = thymine
  modified_base 1251..1253: mod_base = OTHER; note = thymine
  modified_base 1256: mod_base = OTHER; note = thymine
  modified_base 1263..1264: mod_base = OTHER; note = thymine
  modified_base 1276: mod_base = OTHER; note = thymine
  modified_base 1280: mod_base = OTHER; note = thymine
  modified_base 1283: mod_base = OTHER; note = thymine
  modified_base 1292: mod_base = OTHER; note = thymine
  modified_base 1296: mod_base = OTHER; note = thymine
  modified_base 1298: mod_base = OTHER; note = thymine
  modified_base 1300: mod_base = OTHER; note = thymine
  modified_base 1302: mod_base = OTHER; note = thymine
  modified_base 1309: mod_base = OTHER; note = thymine
  modified_base 1313: mod_base = OTHER; note = thymine
  modified_base 1315..1317: mod_base = OTHER; note = thymine
  modified_base 1324: mod_base = OTHER; note = thymine
  modified_base 1326: mod_base = OTHER; note = thymine
  modified_base 1332: mod_base = OTHER; note = thymine
  modified_base 1343: mod_base = OTHER; note = thymine
  modified_base 1346..1347: mod_base = OTHER; note = thymine
  modified_base 1350: mod_base = OTHER; note = thymine
  modified_base 1355: mod_base = OTHER; note = thymine
  modified_base 1357..1358: mod_base = OTHER; note = thymine
  modified_base 1361: mod_base = OTHER; note = thymine
  modified_base 1367..1369: mod_base = OTHER; note = thymine
  modified_base 1377..1378: mod_base = OTHER; note = thymine
  modified_base 1380: mod_base = OTHER; note = thymine
  modified_base 1386: mod_base = OTHER; note = thymine
  modified_base 1388: mod_base = OTHER; note = thymine
  modified_base 1391: mod_base = OTHER; note = thymine
  modified_base 1399..1400: mod_base = OTHER; note = thymine
  modified_base 1404: mod_base = OTHER; note = thymine
  modified_base 1410..1411: mod_base = OTHER; note = thymine
  modified_base 1414: mod_base = OTHER; note = thymine
  modified_base 1421: mod_base = OTHER; note = thymine
  modified_base 1429: mod_base = OTHER; note = thymine
  modified_base 1440: mod_base = OTHER; note = thymine
  modified_base 1442: mod_base = OTHER; note = thymine
  modified_base 1446: mod_base = OTHER; note = thymine
  modified_base 1455: mod_base = OTHER; note = thymine
  modified_base 1461: mod_base = OTHER; note = thymine
  modified_base 1466..1467: mod_base = OTHER; note = thymine
  modified_base 1471: mod_base = OTHER; note = thymine
  modified_base 1473: mod_base = OTHER; note = thymine
  modified_base 1475..1476: mod_base = OTHER; note = thymine
  modified_base 1482: mod_base = OTHER; note = thymine
  modified_base 1484: mod_base = OTHER; note = thymine
  modified_base 1488: mod_base = OTHER; note = thymine
  modified_base 1493..1494: mod_base = OTHER; note = thymine
  modified_base 1497: mod_base = OTHER; note = thymine
  modified_base 1500: mod_base = OTHER; note = thymine
  modified_base 1510: mod_base = OTHER; note = thymine
  modified_base 1516: mod_base = OTHER; note = thymine
  modified_base 1518..1519: mod_base = OTHER; note = thymine
  modified_base 1530: mod_base = OTHER; note = thymine
  modified_base 1532..1533: mod_base = OTHER; note = thymine
  modified_base 1545..1546: mod_base = OTHER; note = thymine
  modified_base 1551: mod_base = OTHER; note = thymine
  modified_base 1557: mod_base = OTHER; note = thymine
  modified_base 1561..1562: mod_base = OTHER; note = thymine
  modified_base 1564..1565: mod_base = OTHER; note = thymine
  modified_base 1571..1572: mod_base = OTHER; note = thymine
  modified_base 1581: mod_base = OTHER; note = thymine
  modified_base 1588..1591: mod_base = OTHER; note = thymine
  modified_base 1596: mod_base = OTHER; note = thymine
  modified_base 1604: mod_base = OTHER; note = thymine
  modified_base 1607..1609: mod_base = OTHER; note = thymine
  modified_base 1613..1614: mod_base = OTHER; note = thymine
  modified_base 1621..1622: mod_base = OTHER; note = thymine
  modified_base 1627: mod_base = OTHER; note = thymine
  modified_base 1637: mod_base = OTHER; note = thymine
  modified_base 1642: mod_base = OTHER; note = thymine
  modified_base 1646..1647: mod_base = OTHER; note = thymine
  modified_base 1657: mod_base = OTHER; note = thymine
  modified_base 1659: mod_base = OTHER; note = thymine
  modified_base 1661: mod_base = OTHER; note = thymine
  modified_base 1678: mod_base = OTHER; note = thymine
  modified_base 1680..1681: mod_base = OTHER; note = thymine
  modified_base 1692: mod_base = OTHER; note = thymine
  modified_base 1696: mod_base = OTHER; note = thymine
  modified_base 1699: mod_base = OTHER; note = thymine
  modified_base 1706..1707: mod_base = OTHER; note = thymine
  modified_base 1710: mod_base = OTHER; note = thymine
  modified_base 1717: mod_base = OTHER; note = thymine
  modified_base 1723..1724: mod_base = OTHER; note = thymine
  modified_base 1732: mod_base = OTHER; note = thymine
  modified_base 1737..1738: mod_base = OTHER; note = thymine
  modified_base 1745: mod_base = OTHER; note = thymine
  modified_base 1750: mod_base = OTHER; note = thymine
  modified_base 1757: mod_base = OTHER; note = thymine
  modified_base 1764: mod_base = OTHER; note = thymine
  modified_base 1767..1769: mod_base = OTHER; note = thymine
  modified_base 1775..1776: mod_base = OTHER; note = thymine
  modified_base 1780: mod_base = OTHER; note = thymine
  modified_base 1784: mod_base = OTHER; note = thymine
  modified_base 1786: mod_base = OTHER; note = thymine
  modified_base 1794: mod_base = OTHER; note = thymine
  modified_base 1799: mod_base = OTHER; note = thymine
  modified_base 1803..1805: mod_base = OTHER; note = thymine
  modified_base 1807: mod_base = OTHER; note = thymine
  modified_base 1816..1817: mod_base = OTHER; note = thymine
  modified_base 1819: mod_base = OTHER; note = thymine
  modified_base 1826: mod_base = OTHER; note = thymine
  modified_base 1828: mod_base = OTHER; note = thymine
  modified_base 1830: mod_base = OTHER; note = thymine
  modified_base 1840: mod_base = OTHER; note = thymine
  modified_base 1842: mod_base = OTHER; note = thymine
  modified_base 1851: mod_base = OTHER; note = thymine
  modified_base 1854: mod_base = OTHER; note = thymine
  modified_base 1857..1859: mod_base = OTHER; note = thymine
  modified_base 1864: mod_base = OTHER; note = thymine
  modified_base 1867: mod_base = OTHER; note = thymine
  modified_base 1875..1876: mod_base = OTHER; note = thymine
  modified_base 1879: mod_base = OTHER; note = thymine
  modified_base 1886..1887: mod_base = OTHER; note = thymine
  modified_base 1892: mod_base = OTHER; note = thymine
  modified_base 1898: mod_base = OTHER; note = thymine
  modified_base 1909: mod_base = OTHER; note = thymine
  modified_base 1912: mod_base = OTHER; note = thymine
  modified_base 1917..1919: mod_base = OTHER; note = thymine
  modified_base 1923: mod_base = OTHER; note = thymine
  modified_base 1925..1926: mod_base = OTHER; note = thymine
  modified_base 1929: mod_base = OTHER; note = thymine
  modified_base 1933: mod_base = OTHER; note = thymine
  modified_base 1936: mod_base = OTHER; note = thymine
  modified_base 1945..1946: mod_base = OTHER; note = thymine
  modified_base 1951: mod_base = OTHER; note = thymine
  modified_base 1954..1955: mod_base = OTHER; note = thymine
  modified_base 1957: mod_base = OTHER; note = thymine
  modified_base 1963..1964: mod_base = OTHER; note = thymine
  modified_base 1976: mod_base = OTHER; note = thymine
  modified_base 1989: mod_base = OTHER; note = thymine
  modified_base 2001: mod_base = OTHER; note = thymine
  modified_base 2010: mod_base = OTHER; note = thymine
  modified_base 2021: mod_base = OTHER; note = thymine
  modified_base 2023..2024: mod_base = OTHER; note = thymine
  modified_base 2027: mod_base = OTHER; note = thymine
  modified_base 2036: mod_base = OTHER; note = thymine
  modified_base 2038..2040: mod_base = OTHER; note = thymine
  modified_base 2046: mod_base = OTHER; note = thymine
  modified_base 2050: mod_base = OTHER; note = thymine
  modified_base 2056: mod_base = OTHER; note = thymine
  modified_base 2060..2061: mod_base = OTHER; note = thymine
  modified_base 2072: mod_base = OTHER; note = thymine
  modified_base 2077..2078: mod_base = OTHER; note = thymine
  modified_base 2081: mod_base = OTHER; note = thymine
  modified_base 2086: mod_base = OTHER; note = thymine
  modified_base 2088: mod_base = OTHER; note = thymine
  modified_base 2091: mod_base = OTHER; note = thymine
  modified_base 2096..2097: mod_base = OTHER; note = thymine
  modified_base 2106: mod_base = OTHER; note = thymine
  modified_base 2108..2109: mod_base = OTHER; note = thymine
  modified_base 2112: mod_base = OTHER; note = thymine
  modified_base 2117..2118: mod_base = OTHER; note = thymine
  modified_base 2120: mod_base = OTHER; note = thymine
  modified_base 2132: mod_base = OTHER; note = thymine
  modified_base 2136..2137: mod_base = OTHER; note = thymine
  modified_base 2140..2142: mod_base = OTHER; note = thymine
  modified_base 2146..2148: mod_base = OTHER; note = thymine
  modified_base 2154..2155: mod_base = OTHER; note = thymine
  modified_base 2163: mod_base = OTHER; note = thymine
  modified_base 2166: mod_base = OTHER; note = thymine
  modified_base 2174: mod_base = OTHER; note = thymine
  modified_base 2178: mod_base = OTHER; note = thymine
  modified_base 2182..2183: mod_base = OTHER; note = thymine
  modified_base 2189..2191: mod_base = OTHER; note = thymine
  modified_base 2195: mod_base = OTHER; note = thymine
  modified_base 2197: mod_base = OTHER; note = thymine
  modified_base 2203..2204: mod_base = OTHER; note = thymine
  modified_base 2209: mod_base = OTHER; note = thymine
  modified_base 2211: mod_base = OTHER; note = thymine
  modified_base 2213: mod_base = OTHER; note = thymine
  modified_base 2224: mod_base = OTHER; note = thymine
  modified_base 2227: mod_base = OTHER; note = thymine
  modified_base 2230: mod_base = OTHER; note = thymine
  modified_base 2233: mod_base = OTHER; note = thymine
  modified_base 2236..2238: mod_base = OTHER; note = thymine
  modified_base 2241: mod_base = OTHER; note = thymine
  modified_base 2245: mod_base = OTHER; note = thymine
  modified_base 2250: mod_base = OTHER; note = thymine
  modified_base 2258: mod_base = OTHER; note = thymine
  modified_base 2260: mod_base = OTHER; note = thymine
  modified_base 2262: mod_base = OTHER; note = thymine
  modified_base 2265: mod_base = OTHER; note = thymine
  modified_base 2268: mod_base = OTHER; note = thymine
  modified_base 2270..2272: mod_base = OTHER; note = thymine
  modified_base 2275: mod_base = OTHER; note = thymine
  modified_base 2284..2286: mod_base = OTHER; note = thymine
  modified_base 2289: mod_base = OTHER; note = thymine
  modified_base 2293: mod_base = OTHER; note = thymine
  modified_base 2299: mod_base = OTHER; note = thymine
  modified_base 2303: mod_base = OTHER; note = thymine
  modified_base 2306: mod_base = OTHER; note = thymine
  modified_base 2310..2311: mod_base = OTHER; note = thymine
  modified_base 2319: mod_base = OTHER; note = thymine
  modified_base 2323..2324: mod_base = OTHER; note = thymine
  modified_base 2330: mod_base = OTHER; note = thymine
  modified_base 2336..2337: mod_base = OTHER; note = thymine
  modified_base 2339: mod_base = OTHER; note = thymine
  modified_base 2343: mod_base = OTHER; note = thymine
  modified_base 2346: mod_base = OTHER; note = thymine
  modified_base 2348: mod_base = OTHER; note = thymine
  modified_base 2350: mod_base = OTHER; note = thymine
  modified_base 2352: mod_base = OTHER; note = thymine
  modified_base 2356..2358: mod_base = OTHER; note = thymine
  modified_base 2365: mod_base = OTHER; note = thymine
  modified_base 2376..2377: mod_base = OTHER; note = thymine
  modified_base 2383: mod_base = OTHER; note = thymine
  modified_base 2387: mod_base = OTHER; note = thymine
  modified_base 2394: mod_base = OTHER; note = thymine
  modified_base 2397: mod_base = OTHER; note = thymine
  modified_base 2410..2412: mod_base = OTHER; note = thymine
  modified_base 2422..2423: mod_base = OTHER; note = thymine
  modified_base 2425: mod_base = OTHER; note = thymine
  modified_base 2427: mod_base = OTHER; note = thymine
  modified_base 2430..2431: mod_base = OTHER; note = thymine
  modified_base 2443..2445: mod_base = OTHER; note = thymine
  modified_base 2452: mod_base = OTHER; note = thymine
  modified_base 2456..2457: mod_base = OTHER; note = thymine
  modified_base 2469: mod_base = OTHER; note = thymine
  modified_base 2478..2480: mod_base = OTHER; note = thymine
  modified_base 2489: mod_base = OTHER; note = thymine
  modified_base 2493: mod_base = OTHER; note = thymine
  modified_base 2499: mod_base = OTHER; note = thymine
  modified_base 2505: mod_base = OTHER; note = thymine
  modified_base 2510: mod_base = OTHER; note = thymine
  modified_base 2513: mod_base = OTHER; note = thymine
  modified_base 2517: mod_base = OTHER; note = thymine
  modified_base 2519..2520: mod_base = OTHER; note = thymine
  modified_base 2529: mod_base = OTHER; note = thymine
  modified_base 2532: mod_base = OTHER; note = thymine
  modified_base 2542..2543: mod_base = OTHER; note = thymine
  modified_base 2550: mod_base = OTHER; note = thymine
  modified_base 2553..2554: mod_base = OTHER; note = thymine
  modified_base 2556..2557: mod_base = OTHER; note = thymine
  modified_base 2560: mod_base = OTHER; note = thymine
  modified_base 2565: mod_base = OTHER; note = thymine
  modified_base 2569: mod_base = OTHER; note = thymine
  modified_base 2585: mod_base = OTHER; note = thymine
  modified_base 2593..2595: mod_base = OTHER; note = thymine
  modified_base 2597..2598: mod_base = OTHER; note = thymine
  modified_base 2602..2603: mod_base = OTHER; note = thymine
  modified_base 2605: mod_base = OTHER; note = thymine
  modified_base 2614..2616: mod_base = OTHER; note = thymine
  modified_base 2621: mod_base = OTHER; note = thymine
  modified_base 2625: mod_base = OTHER; note = thymine
  modified_base 2632..2633: mod_base = OTHER; note = thymine
  modified_base 2636: mod_base = OTHER; note = thymine
  modified_base 2640: mod_base = OTHER; note = thymine
  modified_base 2649..2650: mod_base = OTHER; note = thymine
  modified_base 2654: mod_base = OTHER; note = thymine
  modified_base 2657..2658: mod_base = OTHER; note = thymine
  modified_base 2664: mod_base = OTHER; note = thymine
  modified_base 2666: mod_base = OTHER; note = thymine
  modified_base 2673..2674: mod_base = OTHER; note = thymine
  modified_base 2681: mod_base = OTHER; note = thymine
  modified_base 2683..2684: mod_base = OTHER; note = thymine
  modified_base 2687: mod_base = OTHER; note = thymine
  modified_base 2691: mod_base = OTHER; note = thymine
  modified_base 2694: mod_base = OTHER; note = thymine
  modified_base 2698: mod_base = OTHER; note = thymine
  modified_base 2703: mod_base = OTHER; note = thymine
  modified_base 2709..2711: mod_base = OTHER; note = thymine
  modified_base 2714: mod_base = OTHER; note = thymine
  modified_base 2718: mod_base = OTHER; note = thymine
  modified_base 2720: mod_base = OTHER; note = thymine
  modified_base 2722: mod_base = OTHER; note = thymine
  modified_base 2726: mod_base = OTHER; note = thymine
  modified_base 2732: mod_base = OTHER; note = thymine
  modified_base 2734: mod_base = OTHER; note = thymine
  modified_base 2743..2744: mod_base = OTHER; note = thymine
  modified_base 2747: mod_base = OTHER; note = thymine
  modified_base 2749: mod_base = OTHER; note = thymine
  modified_base 2759: mod_base = OTHER; note = thymine
  modified_base 2761: mod_base = OTHER; note = thymine
  modified_base 2765: mod_base = OTHER; note = thymine
  modified_base 2768: mod_base = OTHER; note = thymine
  modified_base 2772..2774: mod_base = OTHER; note = thymine
  modified_base 2777: mod_base = OTHER; note = thymine
  modified_base 2784..2785: mod_base = OTHER; note = thymine
  modified_base 2797: mod_base = OTHER; note = thymine
  modified_base 2801: mod_base = OTHER; note = thymine
  modified_base 2804: mod_base = OTHER; note = thymine
  modified_base 2813: mod_base = OTHER; note = thymine
  modified_base 2817: mod_base = OTHER; note = thymine
  modified_base 2819: mod_base = OTHER; note = thymine
  modified_base 2821: mod_base = OTHER; note = thymine
  modified_base 2823: mod_base = OTHER; note = thymine
  modified_base 2830: mod_base = OTHER; note = thymine
  modified_base 2834: mod_base = OTHER; note = thymine
  modified_base 2836..2838: mod_base = OTHER; note = thymine
  modified_base 2845: mod_base = OTHER; note = thymine
  modified_base 2847: mod_base = OTHER; note = thymine
  modified_base 2853: mod_base = OTHER; note = thymine
  modified_base 2864: mod_base = OTHER; note = thymine
  modified_base 2867..2868: mod_base = OTHER; note = thymine
  modified_base 2871: mod_base = OTHER; note = thymine
  modified_base 2876: mod_base = OTHER; note = thymine
  modified_base 2878..2879: mod_base = OTHER; note = thymine
  modified_base 2882: mod_base = OTHER; note = thymine
  modified_base 2888..2890: mod_base = OTHER; note = thymine
  modified_base 2898..2899: mod_base = OTHER; note = thymine
  modified_base 2901: mod_base = OTHER; note = thymine
  modified_base 2907: mod_base = OTHER; note = thymine
  modified_base 2909: mod_base = OTHER; note = thymine
  modified_base 2912: mod_base = OTHER; note = thymine
  modified_base 2916: mod_base = OTHER; note = thymine
  modified_base 2920..2921: mod_base = OTHER; note = thymine
  modified_base 2925: mod_base = OTHER; note = thymine
  modified_base 2931..2932: mod_base = OTHER; note = thymine
  modified_base 2935: mod_base = OTHER; note = thymine
  modified_base 2942: mod_base = OTHER; note = thymine
  modified_base 2950: mod_base = OTHER; note = thymine
  modified_base 2961: mod_base = OTHER; note = thymine
  modified_base 2963..2964: mod_base = OTHER; note = thymine
  modified_base 2967: mod_base = OTHER; note = thymine
  modified_base 2976: mod_base = OTHER; note = thymine
  modified_base 2982: mod_base = OTHER; note = thymine
  modified_base 2987..2988: mod_base = OTHER; note = thymine
  modified_base 2992: mod_base = OTHER; note = thymine
  modified_base 2994: mod_base = OTHER; note = thymine
  modified_base 2996..2997: mod_base = OTHER; note = thymine
  modified_base 3003: mod_base = OTHER; note = thymine
  modified_base 3005: mod_base = OTHER; note = thymine
  modified_base 3009: mod_base = OTHER; note = thymine
  modified_base 3014..3015: mod_base = OTHER; note = thymine
  modified_base 3018: mod_base = OTHER; note = thymine
  modified_base 3021: mod_base = OTHER; note = thymine
  modified_base 3031: mod_base = OTHER; note = thymine
  modified_base 3037: mod_base = OTHER; note = thymine
  modified_base 3039..3040: mod_base = OTHER; note = thymine
  modified_base 3050..3052: mod_base = OTHER; note = thymine
  modified_base 3055: mod_base = OTHER; note = thymine
  modified_base 3058: mod_base = OTHER; note = thymine
  modified_base 3062: mod_base = OTHER; note = thymine
  modified_base 3068: mod_base = OTHER; note = thymine
  modified_base 3075: mod_base = OTHER; note = thymine
  modified_base 3086..3087: mod_base = OTHER; note = thymine
  modified_base 3094: mod_base = OTHER; note = thymine
  modified_base 3100: mod_base = OTHER; note = thymine
  modified_base 3117: mod_base = OTHER; note = thymine
  modified_base 3123: mod_base = OTHER; note = thymine
  modified_base 3130: mod_base = OTHER; note = thymine
  modified_base 3148: mod_base = OTHER; note = thymine
  modified_base 3152: mod_base = OTHER; note = thymine
  modified_base 3159: mod_base = OTHER; note = thymine
  modified_base 3161: mod_base = OTHER; note = thymine
  modified_base 3167..3168: mod_base = OTHER; note = thymine
  modified_base 3172: mod_base = OTHER; note = thymine
  modified_base 3174: mod_base = OTHER; note = thymine
  modified_base 3185: mod_base = OTHER; note = thymine
  modified_base 3199: mod_base = OTHER; note = thymine
  modified_base 3205: mod_base = OTHER; note = thymine
  modified_base 3208: mod_base = OTHER; note = thymine
  modified_base 3215: mod_base = OTHER; note = thymine
  modified_base 3226: mod_base = OTHER; note = thymine
  modified_base 3233: mod_base = OTHER; note = thymine
  modified_base 3239: mod_base = OTHER; note = thymine
  modified_base 3241..3243: mod_base = OTHER; note = thymine
  modified_base 3246: mod_base = OTHER; note = thymine
  modified_base 3252..3253: mod_base = OTHER; note = thymine
  modified_base 3256: mod_base = OTHER; note = thymine
  modified_base 3260: mod_base = OTHER; note = thymine
  modified_base 3263: mod_base = OTHER; note = thymine
  modified_base 3267: mod_base = OTHER; note = thymine
  modified_base 3273: mod_base = OTHER; note = thymine
  modified_base 3277..3278: mod_base = OTHER; note = thymine
  modified_base 3282..3283: mod_base = OTHER; note = thymine
  modified_base 3290..3291: mod_base = OTHER; note = thymine
  modified_base 3299: mod_base = OTHER; note = thymine
  modified_base 3321: mod_base = OTHER; note = thymine
  modified_base 3325: mod_base = OTHER; note = thymine
  modified_base 3331..3332: mod_base = OTHER; note = thymine
  modified_base 3334: mod_base = OTHER; note = thymine
  modified_base 3345: mod_base = OTHER; note = thymine
  modified_base 3354..3355: mod_base = OTHER; note = thymine
  modified_base 3358: mod_base = OTHER; note = thymine
  modified_base 3361: mod_base = OTHER; note = thymine
  modified_base 3378: mod_base = OTHER; note = thymine
  modified_base 3381: mod_base = OTHER; note = thymine
  modified_base 3396: mod_base = OTHER; note = thymine
  modified_base 3403: mod_base = OTHER; note = thymine
  modified_base 3405..3406: mod_base = OTHER; note = thymine
  modified_base 3409: mod_base = OTHER; note = thymine
  modified_base 3413: mod_base = OTHER; note = thymine
  modified_base 3425: mod_base = OTHER; note = thymine
  modified_base 3438: mod_base = OTHER; note = thymine
  modified_base 3443: mod_base = OTHER; note = thymine
  modified_base 3450: mod_base = OTHER; note = thymine
  modified_base 3456..3457: mod_base = OTHER; note = thymine
  modified_base 3459: mod_base = OTHER; note = thymine
  modified_base 3461: mod_base = OTHER; note = thymine
  modified_base 3465: mod_base = OTHER; note = thymine
  modified_base 3468: mod_base = OTHER; note = thymine
  modified_base 3474: mod_base = OTHER; note = thymine
  modified_base 3476..3479: mod_base = OTHER; note = thymine
  modified_base 3481: mod_base = OTHER; note = thymine
  modified_base 3489: mod_base = OTHER; note = thymine
  modified_base 3494: mod_base = OTHER; note = thymine
  modified_base 3503: mod_base = OTHER; note = thymine
  modified_base 3505: mod_base = OTHER; note = thymine
  modified_base 3510..3513: mod_base = OTHER; note = thymine
  modified_base 3515: mod_base = OTHER; note = thymine
  modified_base 3517: mod_base = OTHER; note = thymine
  modified_base 3519: mod_base = OTHER; note = thymine
  modified_base 3523..3524: mod_base = OTHER; note = thymine
  modified_base 3526: mod_base = OTHER; note = thymine
  modified_base 3530: mod_base = OTHER; note = thymine
  modified_base 3536: mod_base = OTHER; note = thymine
  modified_base 3541: mod_base = OTHER; note = thymine
  modified_base 3545: mod_base = OTHER; note = thymine
  modified_base 3549: mod_base = OTHER; note = thymine
  modified_base 3564: mod_base = OTHER; note = thymine
  modified_base 3567: mod_base = OTHER; note = thymine
  modified_base 3576: mod_base = OTHER; note = thymine
  modified_base 3584..3585: mod_base = OTHER; note = thymine
  modified_base 3587: mod_base = OTHER; note = thymine
  modified_base 3593..3595: mod_base = OTHER; note = thymine
  modified_base 3597..3598: mod_base = OTHER; note = thymine
  modified_base 3602..3604: mod_base = OTHER; note = thymine
  modified_base 3611: mod_base = OTHER; note = thymine
  modified_base 3615: mod_base = OTHER; note = thymine
  modified_base 3624..3627: mod_base = OTHER; note = thymine
  modified_base 3631: mod_base = OTHER; note = thymine
  modified_base 3638: mod_base = OTHER; note = thymine
  modified_base 3644: mod_base = OTHER; note = thymine
  modified_base 3646: mod_base = OTHER; note = thymine
  modified_base 3648: mod_base = OTHER; note = thymine
  modified_base 3652: mod_base = OTHER; note = thymine
  modified_base 3659: mod_base = OTHER; note = thymine
  modified_base 3664..3665: mod_base = OTHER; note = thymine
  modified_base 3670..3671: mod_base = OTHER; note = thymine
  modified_base 3677: mod_base = OTHER; note = thymine
  modified_base 3685..3686: mod_base = OTHER; note = thymine
  modified_base 3690: mod_base = OTHER; note = thymine
  modified_base 3692: mod_base = OTHER; note = thymine
  modified_base 3694..3696: mod_base = OTHER; note = thymine
  modified_base 3710: mod_base = OTHER; note = thymine
  modified_base 3714: mod_base = OTHER; note = thymine
  modified_base 3717: mod_base = OTHER; note = thymine
  modified_base 3719: mod_base = OTHER; note = thymine
  modified_base 3725: mod_base = OTHER; note = thymine
  modified_base 3730: mod_base = OTHER; note = thymine
  modified_base 3734: mod_base = OTHER; note = thymine
  modified_base 3738: mod_base = OTHER; note = thymine
  modified_base 3740: mod_base = OTHER; note = thymine
  modified_base 3756..3757: mod_base = OTHER; note = thymine
  modified_base 3770: mod_base = OTHER; note = thymine
  modified_base 3780..3781: mod_base = OTHER; note = thymine
  modified_base 3784: mod_base = OTHER; note = thymine
  modified_base 3790: mod_base = OTHER; note = thymine
  modified_base 3793: mod_base = OTHER; note = thymine
  modified_base 3805: mod_base = OTHER; note = thymine
  modified_base 3811: mod_base = OTHER; note = thymine
  modified_base 3821..3822: mod_base = OTHER; note = thymine
  modified_base 3826: mod_base = OTHER; note = thymine
  modified_base 3831: mod_base = OTHER; note = thymine
  modified_base 3834: mod_base = OTHER; note = thymine
  modified_base 3837: mod_base = OTHER; note = thymine
  modified_base 3846: mod_base = OTHER; note = thymine
  modified_base 3853: mod_base = OTHER; note = thymine
  modified_base 3856: mod_base = OTHER; note = thymine
  modified_base 3860: mod_base = OTHER; note = thymine
  modified_base 3864: mod_base = OTHER; note = thymine
  modified_base 3866..3868: mod_base = OTHER; note = thymine
  modified_base 3873: mod_base = OTHER; note = thymine
  modified_base 3878: mod_base = OTHER; note = thymine
  modified_base 3880: mod_base = OTHER; note = thymine
  modified_base 3900..3901: mod_base = OTHER; note = thymine
  modified_base 3905: mod_base = OTHER; note = thymine
  modified_base 3908: mod_base = OTHER; note = thymine
  modified_base 3914: mod_base = OTHER; note = thymine
  modified_base 3921: mod_base = OTHER; note = thymine
  modified_base 3935: mod_base = OTHER; note = thymine
  modified_base 3941: mod_base = OTHER; note = thymine
  modified_base 3950..3951: mod_base = OTHER; note = thymine
  modified_base 3980: mod_base = OTHER; note = thymine
  modified_base 3982: mod_base = OTHER; note = thymine
  modified_base 3985..3987: mod_base = OTHER; note = thymine
  modified_base 3990..3991: mod_base = OTHER; note = thymine
  modified_base 3995: mod_base = OTHER; note = thymine
  modified_base 3998: mod_base = OTHER; note = thymine
  modified_base 4011..4012: mod_base = OTHER; note = thymine
  modified_base 4020..4022: mod_base = OTHER; note = thymine
  modified_base 4030..4031: mod_base = OTHER; note = thymine
  modified_base 4042: mod_base = OTHER; note = thymine
  modified_base 4052..4055: mod_base = OTHER; note = thymine
  modified_base 4057: mod_base = OTHER; note = thymine
  modified_base 4063: mod_base = OTHER; note = thymine
  modified_base 4069..4071: mod_base = OTHER; note = thymine
  modified_base 4078: mod_base = OTHER; note = thymine
  modified_base 4081: mod_base = OTHER; note = thymine
  modified_base 4085: mod_base = OTHER; note = thymine
  modified_base 4088..4089: mod_base = OTHER; note = thymine
  modified_base 4091: mod_base = OTHER; note = thymine
  modified_base 4094: mod_base = OTHER; note = thymine
  modified_base 4099: mod_base = OTHER; note = thymine
  modified_base 4102: mod_base = OTHER; note = thymine
  modified_base 4107: mod_base = OTHER; note = thymine
  modified_base 4112: mod_base = OTHER; note = thymine
  modified_base 4114: mod_base = OTHER; note = thymine
  modified_base 4119..4120: mod_base = OTHER; note = thymine
  modified_base 4124: mod_base = OTHER; note = thymine
  modified_base 4129: mod_base = OTHER; note = thymine
  modified_base 4144..4145: mod_base = OTHER; note = thymine
  modified_base 4151: mod_base = OTHER; note = thymine
  modified_base 4154..4155: mod_base = OTHER; note = thymine
  modified_base 4158: mod_base = OTHER; note = thymine
  modified_base 4165: mod_base = OTHER; note = thymine
  modified_base 4171: mod_base = OTHER; note = thymine
  modified_base 4178: mod_base = OTHER; note = thymine
  modified_base 4180: mod_base = OTHER; note = thymine
  modified_base 4183: mod_base = OTHER; note = thymine
  modified_base 4189: mod_base = OTHER; note = thymine
  modified_base 4197: mod_base = OTHER; note = thymine
  modified_base 4202: mod_base = OTHER; note = thymine
  modified_base 4219: mod_base = OTHER; note = thymine
  modified_base 4224: mod_base = OTHER; note = thymine
  modified_base 4229: mod_base = OTHER; note = thymine
  modified_base 4241..4242: mod_base = OTHER; note = thymine
  modified_base 4246: mod_base = OTHER; note = thymine
  modified_base 4262: mod_base = OTHER; note = thymine
  modified_base 4264..4265: mod_base = OTHER; note = thymine
  modified_base 4270: mod_base = OTHER; note = thymine
  modified_base 4275: mod_base = OTHER; note = thymine
  modified_base 4294: mod_base = OTHER; note = thymine
  modified_base 4311: mod_base = OTHER; note = thymine
  modified_base 4324: mod_base = OTHER; note = thymine
  modified_base 4326: mod_base = OTHER; note = thymine
  modified_base 4330..4331: mod_base = OTHER; note = thymine
  modified_base 4336: mod_base = OTHER; note = thymine
  modified_base 4342: mod_base = OTHER; note = thymine
  modified_base 4344: mod_base = OTHER; note = thymine
  modified_base 4355: mod_base = OTHER; note = thymine
  modified_base 4359: mod_base = OTHER; note = thymine
  modified_base 4365: mod_base = OTHER; note = thymine
  modified_base 4369..4370: mod_base = OTHER; note = thymine
  modified_base 4374: mod_base = OTHER; note = thymine
  modified_base 4377: mod_base = OTHER; note = thymine
  modified_base 4380: mod_base = OTHER; note = thymine
  modified_base 4387: mod_base = OTHER; note = thymine
  modified_base 4394: mod_base = OTHER; note = thymine
  modified_base 4402: mod_base = OTHER; note = thymine
  modified_base 4406: mod_base = OTHER; note = thymine
  modified_base 4410..4411: mod_base = OTHER; note = thymine
  modified_base 4420..4421: mod_base = OTHER; note = thymine
  modified_base 4423: mod_base = OTHER; note = thymine
  modified_base 4436: mod_base = OTHER; note = thymine
  modified_base 4444: mod_base = OTHER; note = thymine
  modified_base 4457: mod_base = OTHER; note = thymine
  modified_base 4461: mod_base = OTHER; note = thymine
  modified_base 4466: mod_base = OTHER; note = thymine
  modified_base 4470: mod_base = OTHER; note = thymine
  modified_base 4474: mod_base = OTHER; note = thymine
  modified_base 4482: mod_base = OTHER; note = thymine
  modified_base 4488: mod_base = OTHER; note = thymine
  modified_base 4493: mod_base = OTHER; note = thymine
  modified_base 4499: mod_base = OTHER; note = thymine
  modified_base 4502: mod_base = OTHER; note = thymine
  modified_base 4506: mod_base = OTHER; note = thymine
  modified_base 4512: mod_base = OTHER; note = thymine
  modified_base 4516: mod_base = OTHER; note = thymine
  modified_base 4519: mod_base = OTHER; note = thymine
  modified_base 4527: mod_base = OTHER; note = thymine
  modified_base 4533: mod_base = OTHER; note = thymine
  modified_base 4543: mod_base = OTHER; note = thymine
  modified_base 4549: mod_base = OTHER; note = thymine
  modified_base 4558: mod_base = OTHER; note = thymine
  modified_base 4561..4563: mod_base = OTHER; note = thymine
  modified_base 4571..4573: mod_base = OTHER; note = thymine
  modified_base 4576: mod_base = OTHER; note = thymine
  modified_base 4579: mod_base = OTHER; note = thymine
  modified_base 4583: mod_base = OTHER; note = thymine
  modified_base 4589: mod_base = OTHER; note = thymine
  modified_base 4596: mod_base = OTHER; note = thymine
  modified_base 4607..4608: mod_base = OTHER; note = thymine
  modified_base 4615: mod_base = OTHER; note = thymine
  modified_base 4621: mod_base = OTHER; note = thymine
  modified_base 4638: mod_base = OTHER; note = thymine
  modified_base 4644: mod_base = OTHER; note = thymine
  modified_base 4651: mod_base = OTHER; note = thymine
  modified_base 4669: mod_base = OTHER; note = thymine
  modified_base 4673: mod_base = OTHER; note = thymine
  modified_base 4680: mod_base = OTHER; note = thymine
  modified_base 4682: mod_base = OTHER; note = thymine
  modified_base 4688..4689: mod_base = OTHER; note = thymine
  modified_base 4693: mod_base = OTHER; note = thymine
  modified_base 4695: mod_base = OTHER; note = thymine
  modified_base 4706: mod_base = OTHER; note = thymine
  modified_base 4720: mod_base = OTHER; note = thymine
  modified_base 4726: mod_base = OTHER; note = thymine
  modified_base 4729: mod_base = OTHER; note = thymine
  modified_base 4736: mod_base = OTHER; note = thymine
  modified_base 4747: mod_base = OTHER; note = thymine
  modified_base 4754: mod_base = OTHER; note = thymine
  modified_base 4760: mod_base = OTHER; note = thymine
  modified_base 4762..4764: mod_base = OTHER; note = thymine
  modified_base 4767: mod_base = OTHER; note = thymine
  modified_base 4773..4774: mod_base = OTHER; note = thymine
  modified_base 4777: mod_base = OTHER; note = thymine
  modified_base 4781: mod_base = OTHER; note = thymine
  modified_base 4784: mod_base = OTHER; note = thymine
  modified_base 4788: mod_base = OTHER; note = thymine
  modified_base 4794: mod_base = OTHER; note = thymine
  modified_base 4798..4799: mod_base = OTHER; note = thymine
  modified_base 4803..4804: mod_base = OTHER; note = thymine
  modified_base 4811..4812: mod_base = OTHER; note = thymine
  modified_base 4820: mod_base = OTHER; note = thymine
  modified_base 4842: mod_base = OTHER; note = thymine
  modified_base 4846: mod_base = OTHER; note = thymine
  modified_base 4852..4853: mod_base = OTHER; note = thymine
  modified_base 4855: mod_base = OTHER; note = thymine
  modified_base 4866: mod_base = OTHER; note = thymine
  modified_base 4875..4876: mod_base = OTHER; note = thymine
  modified_base 4879: mod_base = OTHER; note = thymine
  modified_base 4882: mod_base = OTHER; note = thymine
  modified_base 4899: mod_base = OTHER; note = thymine
  modified_base 4902: mod_base = OTHER; note = thymine
  modified_base 4917: mod_base = OTHER; note = thymine
  modified_base 4924: mod_base = OTHER; note = thymine
  modified_base 4926..4927: mod_base = OTHER; note = thymine
  modified_base 4930: mod_base = OTHER; note = thymine
  modified_base 4934: mod_base = OTHER; note = thymine
  modified_base 4946: mod_base = OTHER; note = thymine
  modified_base 4959: mod_base = OTHER; note = thymine
  modified_base 4964: mod_base = OTHER; note = thymine
  modified_base 4971: mod_base = OTHER; note = thymine
  modified_base 4977..4978: mod_base = OTHER; note = thymine
  modified_base 4980: mod_base = OTHER; note = thymine
  modified_base 4982: mod_base = OTHER; note = thymine
  modified_base 4986: mod_base = OTHER; note = thymine
  modified_base 4989: mod_base = OTHER; note = thymine
  modified_base 4995: mod_base = OTHER; note = thymine
  modified_base 4997..5000: mod_base = OTHER; note = thymine
  modified_base 5002: mod_base = OTHER; note = thymine
  modified_base 5010: mod_base = OTHER; note = thymine
  modified_base 5015: mod_base = OTHER; note = thymine
  modified_base 5024: mod_base = OTHER; note = thymine
  modified_base 5026: mod_base = OTHER; note = thymine
  modified_base 5031..5034: mod_base = OTHER; note = thymine
  modified_base 5036: mod_base = OTHER; note = thymine
  modified_base 5038: mod_base = OTHER; note = thymine
  modified_base 5040: mod_base = OTHER; note = thymine
  modified_base 5044..5045: mod_base = OTHER; note = thymine
  modified_base 5047: mod_base = OTHER; note = thymine
  modified_base 5051: mod_base = OTHER; note = thymine
  modified_base 5057: mod_base = OTHER; note = thymine
  modified_base 5062: mod_base = OTHER; note = thymine
  modified_base 5066: mod_base = OTHER; note = thymine
  modified_base 5070: mod_base = OTHER; note = thymine
  modified_base 5085: mod_base = OTHER; note = thymine
  modified_base 5088: mod_base = OTHER; note = thymine
  modified_base 5097: mod_base = OTHER; note = thymine
  modified_base 5105..5106: mod_base = OTHER; note = thymine
  modified_base 5108: mod_base = OTHER; note = thymine
  modified_base 5114..5116: mod_base = OTHER; note = thymine
  modified_base 5118..5119: mod_base = OTHER; note = thymine
  modified_base 5123..5125: mod_base = OTHER; note = thymine
  modified_base 5132: mod_base = OTHER; note = thymine
  modified_base 5136: mod_base = OTHER; note = thymine
  modified_base 5145..5148: mod_base = OTHER; note = thymine
  modified_base 5152: mod_base = OTHER; note = thymine
  modified_base 5159: mod_base = OTHER; note = thymine
  modified_base 5165: mod_base = OTHER; note = thymine
  modified_base 5167: mod_base = OTHER; note = thymine
  modified_base 5169: mod_base = OTHER; note = thymine
  modified_base 5173: mod_base = OTHER; note = thymine
  modified_base 5180: mod_base = OTHER; note = thymine
  modified_base 5185..5186: mod_base = OTHER; note = thymine
  modified_base 5191..5192: mod_base = OTHER; note = thymine
  modified_base 5198: mod_base = OTHER; note = thymine
  modified_base 5206..5207: mod_base = OTHER; note = thymine
  modified_base 5211: mod_base = OTHER; note = thymine
  modified_base 5213: mod_base = OTHER; note = thymine
  modified_base 5215..5217: mod_base = OTHER; note = thymine
  modified_base 5231: mod_base = OTHER; note = thymine
  modified_base 5235: mod_base = OTHER; note = thymine
  modified_base 5238: mod_base = OTHER; note = thymine
  modified_base 5240: mod_base = OTHER; note = thymine
  modified_base 5246: mod_base = OTHER; note = thymine
  modified_base 5251: mod_base = OTHER; note = thymine
  modified_base 5255: mod_base = OTHER; note = thymine
  modified_base 5259: mod_base = OTHER; note = thymine
  modified_base 5261: mod_base = OTHER; note = thymine
  modified_base 5277..5278: mod_base = OTHER; note = thymine
  modified_base 5291: mod_base = OTHER; note = thymine
  modified_base 5301..5302: mod_base = OTHER; note = thymine
  modified_base 5305: mod_base = OTHER; note = thymine
  modified_base 5311: mod_base = OTHER; note = thymine
  modified_base 5314: mod_base = OTHER; note = thymine
  modified_base 5326: mod_base = OTHER; note = thymine
  modified_base 5332: mod_base = OTHER; note = thymine
  modified_base 5342..5343: mod_base = OTHER; note = thymine
  modified_base 5347: mod_base = OTHER; note = thymine
  modified_base 5352: mod_base = OTHER; note = thymine
  modified_base 5355: mod_base = OTHER; note = thymine
  modified_base 5358: mod_base = OTHER; note = thymine
  modified_base 5367: mod_base = OTHER; note = thymine
  modified_base 5374: mod_base = OTHER; note = thymine
  modified_base 5377: mod_base = OTHER; note = thymine
  modified_base 5381: mod_base = OTHER; note = thymine
  modified_base 5385: mod_base = OTHER; note = thymine
  modified_base 5387..5389: mod_base = OTHER; note = thymine
  modified_base 5394: mod_base = OTHER; note = thymine
  modified_base 5399: mod_base = OTHER; note = thymine
  modified_base 5401: mod_base = OTHER; note = thymine
  modified_base 5421..5422: mod_base = OTHER; note = thymine
  modified_base 5426: mod_base = OTHER; note = thymine
  modified_base 5429: mod_base = OTHER; note = thymine
  modified_base 5435: mod_base = OTHER; note = thymine
  modified_base 5442: mod_base = OTHER; note = thymine
  modified_base 5456: mod_base = OTHER; note = thymine
  modified_base 5462: mod_base = OTHER; note = thymine
  modified_base 5471..5472: mod_base = OTHER; note = thymine
  modified_base 5501: mod_base = OTHER; note = thymine
  modified_base 5503: mod_base = OTHER; note = thymine
  modified_base 5506..5508: mod_base = OTHER; note = thymine
  modified_base 5511..5512: mod_base = OTHER; note = thymine
  modified_base 5516: mod_base = OTHER; note = thymine
  modified_base 5519: mod_base = OTHER; note = thymine
  modified_base 5532..5533: mod_base = OTHER; note = thymine
  modified_base 5541..5543: mod_base = OTHER; note = thymine
  modified_base 5551..5552: mod_base = OTHER; note = thymine
  modified_base 5563: mod_base = OTHER; note = thymine
  modified_base 5573..5576: mod_base = OTHER; note = thymine
  modified_base 5578: mod_base = OTHER; note = thymine
  modified_base 5584: mod_base = OTHER; note = thymine
  modified_base 5590..5592: mod_base = OTHER; note = thymine
  modified_base 5599: mod_base = OTHER; note = thymine
  modified_base 5602: mod_base = OTHER; note = thymine
  modified_base 5606: mod_base = OTHER; note = thymine
  modified_base 5609..5610: mod_base = OTHER; note = thymine
  modified_base 5612: mod_base = OTHER; note = thymine
  modified_base 5615: mod_base = OTHER; note = thymine
  modified_base 5620: mod_base = OTHER; note = thymine
  modified_base 5623: mod_base = OTHER; note = thymine
  modified_base 5628: mod_base = OTHER; note = thymine
  modified_base 5633: mod_base = OTHER; note = thymine
  modified_base 5635: mod_base = OTHER; note = thymine
  modified_base 5640..5641: mod_base = OTHER; note = thymine
  modified_base 5645: mod_base = OTHER; note = thymine
  modified_base 5650: mod_base = OTHER; note = thymine
  modified_base 5665..5666: mod_base = OTHER; note = thymine
  modified_base 5672: mod_base = OTHER; note = thymine
  modified_base 5675..5676: mod_base = OTHER; note = thymine
  modified_base 5679: mod_base = OTHER; note = thymine
  modified_base 5686: mod_base = OTHER; note = thymine
  modified_base 5692: mod_base = OTHER; note = thymine
  modified_base 5699: mod_base = OTHER; note = thymine
  modified_base 5701: mod_base = OTHER; note = thymine
  modified_base 5704: mod_base = OTHER; note = thymine
  modified_base 5710: mod_base = OTHER; note = thymine
  modified_base 5718: mod_base = OTHER; note = thymine
  modified_base 5723: mod_base = OTHER; note = thymine
  modified_base 5740: mod_base = OTHER; note = thymine
  modified_base 5745: mod_base = OTHER; note = thymine
  modified_base 5750: mod_base = OTHER; note = thymine
  modified_base 5762..5763: mod_base = OTHER; note = thymine
  modified_base 5767: mod_base = OTHER; note = thymine
  modified_base 5783: mod_base = OTHER; note = thymine
  modified_base 5785..5786: mod_base = OTHER; note = thymine
  modified_base 5791: mod_base = OTHER; note = thymine
  modified_base 5796: mod_base = OTHER; note = thymine
  modified_base 5815: mod_base = OTHER; note = thymine
  modified_base 5832: mod_base = OTHER; note = thymine
  modified_base 5845: mod_base = OTHER; note = thymine
  modified_base 5847: mod_base = OTHER; note = thymine
  modified_base 5851..5852: mod_base = OTHER; note = thymine
  modified_base 5857: mod_base = OTHER; note = thymine
  modified_base 5863: mod_base = OTHER; note = thymine
  modified_base 5865: mod_base = OTHER; note = thymine
  modified_base 5876: mod_base = OTHER; note = thymine
  modified_base 5880: mod_base = OTHER; note = thymine
  modified_base 5886: mod_base = OTHER; note = thymine
  modified_base 5890..5891: mod_base = OTHER; note = thymine
  modified_base 5895: mod_base = OTHER; note = thymine
  modified_base 5898: mod_base = OTHER; note = thymine
  modified_base 5901: mod_base = OTHER; note = thymine
  modified_base 5908: mod_base = OTHER; note = thymine
  modified_base 5915: mod_base = OTHER; note = thymine
  modified_base 5923: mod_base = OTHER; note = thymine
  modified_base 5927: mod_base = OTHER; note = thymine
  modified_base 5931..5932: mod_base = OTHER; note = thymine
  modified_base 5941..5942: mod_base = OTHER; note = thymine
  modified_base 5944: mod_base = OTHER; note = thymine
  modified_base 5957: mod_base = OTHER; note = thymine
  modified_base 5965: mod_base = OTHER; note = thymine
  modified_base 5978: mod_base = OTHER; note = thymine
  modified_base 5982: mod_base = OTHER; note = thymine
  modified_base 5987: mod_base = OTHER; note = thymine
  modified_base 5991: mod_base = OTHER; note = thymine
  modified_base 6003: mod_base = OTHER; note = thymine
  modified_base 6009: mod_base = OTHER; note = thymine
  modified_base 6014: mod_base = OTHER; note = thymine
  modified_base 6020: mod_base = OTHER; note = thymine
  modified_base 6023: mod_base = OTHER; note = thymine
  modified_base 6027: mod_base = OTHER; note = thymine
  modified_base 6033: mod_base = OTHER; note = thymine
  modified_base 6037: mod_base = OTHER; note = thymine
  modified_base 6040: mod_base = OTHER; note = thymine
  modified_base 6048: mod_base = OTHER; note = thymine
  modified_base 6054: mod_base = OTHER; note = thymine
  modified_base 6064: mod_base = OTHER; note = thymine
  modified_base 6070: mod_base = OTHER; note = thymine
  modified_base 6079: mod_base = OTHER; note = thymine
  modified_base 6082..6084: mod_base = OTHER; note = thymine
  modified_base 6092..6094: mod_base = OTHER; note = thymine
  modified_base 6097: mod_base = OTHER; note = thymine
  modified_base 6100: mod_base = OTHER; note = thymine
  modified_base 6104: mod_base = OTHER; note = thymine
  modified_base 6110: mod_base = OTHER; note = thymine
  modified_base 6117: mod_base = OTHER; note = thymine
  modified_base 6128..6129: mod_base = OTHER; note = thymine
  modified_base 6136: mod_base = OTHER; note = thymine
  modified_base 6142: mod_base = OTHER; note = thymine
  modified_base 6159: mod_base = OTHER; note = thymine
  modified_base 6165: mod_base = OTHER; note = thymine
  modified_base 6172: mod_base = OTHER; note = thymine
  modified_base 6190: mod_base = OTHER; note = thymine
  modified_base 6194: mod_base = OTHER; note = thymine
  modified_base 6201: mod_base = OTHER; note = thymine
  modified_base 6203: mod_base = OTHER; note = thymine
  modified_base 6209..6210: mod_base = OTHER; note = thymine
  modified_base 6214: mod_base = OTHER; note = thymine
  modified_base 6216: mod_base = OTHER; note = thymine
  modified_base 6227: mod_base = OTHER; note = thymine
  modified_base 6241: mod_base = OTHER; note = thymine
  modified_base 6247: mod_base = OTHER; note = thymine
  modified_base 6250: mod_base = OTHER; note = thymine
  modified_base 6257: mod_base = OTHER; note = thymine
  modified_base 6268: mod_base = OTHER; note = thymine
  modified_base 6275: mod_base = OTHER; note = thymine
  modified_base 6281: mod_base = OTHER; note = thymine
  modified_base 6283..6285: mod_base = OTHER; note = thymine
  modified_base 6288: mod_base = OTHER; note = thymine
  modified_base 6294..6295: mod_base = OTHER; note = thymine
  modified_base 6298: mod_base = OTHER; note = thymine
  modified_base 6302: mod_base = OTHER; note = thymine
  modified_base 6305: mod_base = OTHER; note = thymine
  modified_base 6309: mod_base = OTHER; note = thymine
  modified_base 6315: mod_base = OTHER; note = thymine
  modified_base 6319..6320: mod_base = OTHER; note = thymine
  modified_base 6324..6325: mod_base = OTHER; note = thymine
  modified_base 6332..6333: mod_base = OTHER; note = thymine
  modified_base 6341: mod_base = OTHER; note = thymine
  modified_base 6363: mod_base = OTHER; note = thymine
  modified_base 6367: mod_base = OTHER; note = thymine
  modified_base 6376: mod_base = OTHER; note = thymine
  modified_base 6379..6380: mod_base = OTHER; note = thymine
  modified_base 6382: mod_base = OTHER; note = thymine
  modified_base 6393: mod_base = OTHER; note = thymine
  modified_base 6402..6403: mod_base = OTHER; note = thymine
  modified_base 6406: mod_base = OTHER; note = thymine
  modified_base 6409: mod_base = OTHER; note = thymine
  modified_base 6426: mod_base = OTHER; note = thymine
  modified_base 6429: mod_base = OTHER; note = thymine
  modified_base 6444: mod_base = OTHER; note = thymine
  modified_base 6451: mod_base = OTHER; note = thymine
  modified_base 6453..6454: mod_base = OTHER; note = thymine
  modified_base 6457: mod_base = OTHER; note = thymine
  modified_base 6461: mod_base = OTHER; note = thymine
  modified_base 6473: mod_base = OTHER; note = thymine
  modified_base 6486: mod_base = OTHER; note = thymine
  modified_base 6491: mod_base = OTHER; note = thymine
  modified_base 6498: mod_base = OTHER; note = thymine
  modified_base 6504..6505: mod_base = OTHER; note = thymine
  modified_base 6507: mod_base = OTHER; note = thymine
  modified_base 6509: mod_base = OTHER; note = thymine
  modified_base 6513: mod_base = OTHER; note = thymine
  modified_base 6516: mod_base = OTHER; note = thymine
  modified_base 6522: mod_base = OTHER; note = thymine
  modified_base 6524..6527: mod_base = OTHER; note = thymine
  modified_base 6529: mod_base = OTHER; note = thymine
  modified_base 6537: mod_base = OTHER; note = thymine
  modified_base 6542: mod_base = OTHER; note = thymine
  modified_base 6551: mod_base = OTHER; note = thymine
  modified_base 6553: mod_base = OTHER; note = thymine
  modified_base 6558..6561: mod_base = OTHER; note = thymine
  modified_base 6563: mod_base = OTHER; note = thymine
  modified_base 6565: mod_base = OTHER; note = thymine
  modified_base 6567: mod_base = OTHER; note = thymine
  modified_base 6571..6572: mod_base = OTHER; note = thymine
  modified_base 6574: mod_base = OTHER; note = thymine
  modified_base 6578: mod_base = OTHER; note = thymine
  modified_base 6584: mod_base = OTHER; note = thymine
  modified_base 6589: mod_base = OTHER; note = thymine
  modified_base 6593: mod_base = OTHER; note = thymine
  modified_base 6597: mod_base = OTHER; note = thymine
  modified_base 6612: mod_base = OTHER; note = thymine
  modified_base 6615: mod_base = OTHER; note = thymine
  modified_base 6624: mod_base = OTHER; note = thymine
  modified_base 6632..6633: mod_base = OTHER; note = thymine
  modified_base 6635: mod_base = OTHER; note = thymine
  modified_base 6641..6643: mod_base = OTHER; note = thymine
  modified_base 6645..6646: mod_base = OTHER; note = thymine
  modified_base 6650..6652: mod_base = OTHER; note = thymine
  modified_base 6659: mod_base = OTHER; note = thymine
  modified_base 6663: mod_base = OTHER; note = thymine
  modified_base 6672..6675: mod_base = OTHER; note = thymine
  modified_base 6679: mod_base = OTHER; note = thymine
  modified_base 6686: mod_base = OTHER; note = thymine
  modified_base 6692: mod_base = OTHER; note = thymine
  modified_base 6694: mod_base = OTHER; note = thymine
  modified_base 6696: mod_base = OTHER; note = thymine
  modified_base 6700: mod_base = OTHER; note = thymine
  modified_base 6707: mod_base = OTHER; note = thymine
  modified_base 6712..6713: mod_base = OTHER; note = thymine
  modified_base 6718..6719: mod_base = OTHER; note = thymine
  modified_base 6725: mod_base = OTHER; note = thymine
  modified_base 6733..6734: mod_base = OTHER; note = thymine
  modified_base 6738: mod_base = OTHER; note = thymine
  modified_base 6740: mod_base = OTHER; note = thymine
  modified_base 6742..6744: mod_base = OTHER; note = thymine
  modified_base 6758: mod_base = OTHER; note = thymine
  modified_base 6762: mod_base = OTHER; note = thymine
  modified_base 6765: mod_base = OTHER; note = thymine
  modified_base 6767: mod_base = OTHER; note = thymine
  modified_base 6773: mod_base = OTHER; note = thymine
  modified_base 6778: mod_base = OTHER; note = thymine
  modified_base 6782: mod_base = OTHER; note = thymine
  modified_base 6786: mod_base = OTHER; note = thymine
  modified_base 6788: mod_base = OTHER; note = thymine
  modified_base 6804..6805: mod_base = OTHER; note = thymine
  modified_base 6818: mod_base = OTHER; note = thymine
  modified_base 6828..6829: mod_base = OTHER; note = thymine
  modified_base 6832: mod_base = OTHER; note = thymine
  modified_base 6838: mod_base = OTHER; note = thymine
  modified_base 6841: mod_base = OTHER; note = thymine
  modified_base 6853: mod_base = OTHER; note = thymine
  modified_base 6859: mod_base = OTHER; note = thymine
  modified_base 6869..6870: mod_base = OTHER; note = thymine
  modified_base 6874: mod_base = OTHER; note = thymine
  modified_base 6879: mod_base = OTHER; note = thymine
  modified_base 6882: mod_base = OTHER; note = thymine
  modified_base 6885: mod_base = OTHER; note = thymine
  modified_base 6894: mod_base = OTHER; note = thymine
  modified_base 6901: mod_base = OTHER; note = thymine
  modified_base 6904: mod_base = OTHER; note = thymine
  modified_base 6908: mod_base = OTHER; note = thymine
  modified_base 6912: mod_base = OTHER; note = thymine
  modified_base 6914..6916: mod_base = OTHER; note = thymine
  modified_base 6921: mod_base = OTHER; note = thymine
  modified_base 6926: mod_base = OTHER; note = thymine
  modified_base 6928: mod_base = OTHER; note = thymine
  modified_base 6948..6949: mod_base = OTHER; note = thymine
  modified_base 6953: mod_base = OTHER; note = thymine
  modified_base 6956: mod_base = OTHER; note = thymine
  modified_base 6962: mod_base = OTHER; note = thymine
  modified_base 6969: mod_base = OTHER; note = thymine
  modified_base 6983: mod_base = OTHER; note = thymine
  modified_base 6989: mod_base = OTHER; note = thymine
  modified_base 6998..6999: mod_base = OTHER; note = thymine
  modified_base 7028: mod_base = OTHER; note = thymine
  modified_base 7030: mod_base = OTHER; note = thymine
  modified_base 7033..7035: mod_base = OTHER; note = thymine
  modified_base 7038..7039: mod_base = OTHER; note = thymine
  modified_base 7043: mod_base = OTHER; note = thymine
  modified_base 7046: mod_base = OTHER; note = thymine
  modified_base 7059..7060: mod_base = OTHER; note = thymine
  modified_base 7068..7070: mod_base = OTHER; note = thymine
  modified_base 7078..7079: mod_base = OTHER; note = thymine
  modified_base 7090: mod_base = OTHER; note = thymine
  modified_base 7100..7103: mod_base = OTHER; note = thymine
  modified_base 7105: mod_base = OTHER; note = thymine
  modified_base 7111: mod_base = OTHER; note = thymine
  modified_base 7117..7119: mod_base = OTHER; note = thymine
  modified_base 7126: mod_base = OTHER; note = thymine
  modified_base 7129: mod_base = OTHER; note = thymine
  modified_base 7133: mod_base = OTHER; note = thymine
  modified_base 7136..7137: mod_base = OTHER; note = thymine
  modified_base 7139: mod_base = OTHER; note = thymine
  modified_base 7142: mod_base = OTHER; note = thymine
  modified_base 7147: mod_base = OTHER; note = thymine
  modified_base 7150: mod_base = OTHER; note = thymine
  modified_base 7155: mod_base = OTHER; note = thymine
  modified_base 7160: mod_base = OTHER; note = thymine
  modified_base 7162: mod_base = OTHER; note = thymine
  modified_base 7167..7168: mod_base = OTHER; note = thymine
  modified_base 7172: mod_base = OTHER; note = thymine
  modified_base 7177: mod_base = OTHER; note = thymine
  modified_base 7192..7193: mod_base = OTHER; note = thymine
  modified_base 7199: mod_base = OTHER; note = thymine
  modified_base 7202..7203: mod_base = OTHER; note = thymine
  modified_base 7206: mod_base = OTHER; note = thymine
  modified_base 7213: mod_base = OTHER; note = thymine
  modified_base 7219: mod_base = OTHER; note = thymine
  modified_base 7226: mod_base = OTHER; note = thymine
  modified_base 7228: mod_base = OTHER; note = thymine
  modified_base 7231: mod_base = OTHER; note = thymine
  modified_base 7237: mod_base = OTHER; note = thymine
  modified_base 7245: mod_base = OTHER; note = thymine
  modified_base 7250: mod_base = OTHER; note = thymine
  modified_base 7267: mod_base = OTHER; note = thymine
  modified_base 7272: mod_base = OTHER; note = thymine
  modified_base 7277: mod_base = OTHER; note = thymine
  modified_base 7289..7290: mod_base = OTHER; note = thymine
  modified_base 7294: mod_base = OTHER; note = thymine
  modified_base 7310: mod_base = OTHER; note = thymine
  modified_base 7312..7313: mod_base = OTHER; note = thymine
  modified_base 7318: mod_base = OTHER; note = thymine
  modified_base 7323: mod_base = OTHER; note = thymine
  modified_base 7342: mod_base = OTHER; note = thymine
  modified_base 7359: mod_base = OTHER; note = thymine
  modified_base 7372: mod_base = OTHER; note = thymine
  modified_base 7374: mod_base = OTHER; note = thymine
  modified_base 7378..7379: mod_base = OTHER; note = thymine
  modified_base 7384: mod_base = OTHER; note = thymine
  modified_base 7390: mod_base = OTHER; note = thymine
  modified_base 7392: mod_base = OTHER; note = thymine
  modified_base 7403: mod_base = OTHER; note = thymine
  modified_base 7407: mod_base = OTHER; note = thymine
  modified_base 7413: mod_base = OTHER; note = thymine
  modified_base 7417..7418: mod_base = OTHER; note = thymine
  modified_base 7422: mod_base = OTHER; note = thymine
  modified_base 7425: mod_base = OTHER; note = thymine
  modified_base 7428: mod_base = OTHER; note = thymine
  modified_base 7435: mod_base = OTHER; note = thymine
  modified_base 7442: mod_base = OTHER; note = thymine
  modified_base 7450: mod_base = OTHER; note = thymine
  modified_base 7454: mod_base = OTHER; note = thymine
  modified_base 7458..7459: mod_base = OTHER; note = thymine
  modified_base 7468..7469: mod_base = OTHER; note = thymine
  modified_base 7471: mod_base = OTHER; note = thymine
  modified_base 7484: mod_base = OTHER; note = thymine
  modified_base 7492: mod_base = OTHER; note = thymine
  modified_base 7505: mod_base = OTHER; note = thymine
  modified_base 7509: mod_base = OTHER; note = thymine
  modified_base 7514: mod_base = OTHER; note = thymine
  modified_base 7518: mod_base = OTHER; note = thymine
  modified_base 7522: mod_base = OTHER; note = thymine
  modified_base 7530: mod_base = OTHER; note = thymine
  modified_base 7536: mod_base = OTHER; note = thymine
  modified_base 7541: mod_base = OTHER; note = thymine
  modified_base 7547: mod_base = OTHER; note = thymine
  modified_base 7550: mod_base = OTHER; note = thymine
  modified_base 7554: mod_base = OTHER; note = thymine
  modified_base 7560: mod_base = OTHER; note = thymine
  modified_base 7564: mod_base = OTHER; note = thymine
  modified_base 7567: mod_base = OTHER; note = thymine
  modified_base 7575: mod_base = OTHER; note = thymine
  modified_base 7581: mod_base = OTHER; note = thymine
  modified_base 7591: mod_base = OTHER; note = thymine
  modified_base 7597: mod_base = OTHER; note = thymine
  modified_base 7606: mod_base = OTHER; note = thymine
  modified_base 7609..7611: mod_base = OTHER; note = thymine
  modified_base 7620: mod_base = OTHER; note = thymine
  modified_base 7622..7623: mod_base = OTHER; note = thymine
  modified_base 7635..7636: mod_base = OTHER; note = thymine
  modified_base 7641: mod_base = OTHER; note = thymine
  modified_base 7647: mod_base = OTHER; note = thymine
  modified_base 7651..7652: mod_base = OTHER; note = thymine
  modified_base 7654..7655: mod_base = OTHER; note = thymine
  modified_base 7661..7662: mod_base = OTHER; note = thymine
  modified_base 7671: mod_base = OTHER; note = thymine
  modified_base 7678..7681: mod_base = OTHER; note = thymine
  modified_base 7686: mod_base = OTHER; note = thymine
  modified_base 7694: mod_base = OTHER; note = thymine
  modified_base 7697..7699: mod_base = OTHER; note = thymine
  modified_base 7703..7704: mod_base = OTHER; note = thymine
  modified_base 7711..7712: mod_base = OTHER; note = thymine
  modified_base 7717: mod_base = OTHER; note = thymine
  modified_base 7727: mod_base = OTHER; note = thymine
  modified_base 7732: mod_base = OTHER; note = thymine
  modified_base 7736..7737: mod_base = OTHER; note = thymine
  modified_base 7747: mod_base = OTHER; note = thymine
  modified_base 7749: mod_base = OTHER; note = thymine
  modified_base 7751: mod_base = OTHER; note = thymine
  modified_base 7768: mod_base = OTHER; note = thymine
  modified_base 7770..7771: mod_base = OTHER; note = thymine
  modified_base 7782: mod_base = OTHER; note = thymine
  modified_base 7786: mod_base = OTHER; note = thymine
  modified_base 7789: mod_base = OTHER; note = thymine
  modified_base 7796..7797: mod_base = OTHER; note = thymine
  modified_base 7800: mod_base = OTHER; note = thymine
  modified_base 7807: mod_base = OTHER; note = thymine
  modified_base 7813..7814: mod_base = OTHER; note = thymine
  modified_base 7822: mod_base = OTHER; note = thymine
  modified_base 7827..7828: mod_base = OTHER; note = thymine
  modified_base 7835: mod_base = OTHER; note = thymine
  modified_base 7840: mod_base = OTHER; note = thymine
  modified_base 7847: mod_base = OTHER; note = thymine
  modified_base 7854: mod_base = OTHER; note = thymine
  modified_base 7857..7859: mod_base = OTHER; note = thymine
  modified_base 7865..7866: mod_base = OTHER; note = thymine
  modified_base 7870: mod_base = OTHER; note = thymine
  modified_base 7874: mod_base = OTHER; note = thymine
  modified_base 7876: mod_base = OTHER; note = thymine
  modified_base 7884: mod_base = OTHER; note = thymine
  modified_base 7889: mod_base = OTHER; note = thymine
  modified_base 7893..7895: mod_base = OTHER; note = thymine
  modified_base 7897: mod_base = OTHER; note = thymine
  modified_base 7906..7907: mod_base = OTHER; note = thymine
  modified_base 7909: mod_base = OTHER; note = thymine
  modified_base 7916: mod_base = OTHER; note = thymine
  modified_base 7918: mod_base = OTHER; note = thymine
  modified_base 7920: mod_base = OTHER; note = thymine
  modified_base 7930: mod_base = OTHER; note = thymine
  modified_base 7932: mod_base = OTHER; note = thymine
  modified_base 7941: mod_base = OTHER; note = thymine
  modified_base 7944: mod_base = OTHER; note = thymine
  modified_base 7947..7949: mod_base = OTHER; note = thymine
  modified_base 7954: mod_base = OTHER; note = thymine
  modified_base 7957: mod_base = OTHER; note = thymine
  modified_base 7965..7966: mod_base = OTHER; note = thymine
  modified_base 7969: mod_base = OTHER; note = thymine
  modified_base 7976..7977: mod_base = OTHER; note = thymine
  modified_base 7982: mod_base = OTHER; note = thymine
  modified_base 7988: mod_base = OTHER; note = thymine
  modified_base 7999: mod_base = OTHER; note = thymine
  modified_base 8002: mod_base = OTHER; note = thymine
  modified_base 8007..8009: mod_base = OTHER; note = thymine
  modified_base 8013: mod_base = OTHER; note = thymine
  modified_base 8015..8016: mod_base = OTHER; note = thymine
  modified_base 8019: mod_base = OTHER; note = thymine
  modified_base 8023: mod_base = OTHER; note = thymine
  modified_base 8026: mod_base = OTHER; note = thymine
  modified_base 8035..8036: mod_base = OTHER; note = thymine
  modified_base 8041: mod_base = OTHER; note = thymine
  modified_base 8044..8045: mod_base = OTHER; note = thymine
  modified_base 8047: mod_base = OTHER; note = thymine
  modified_base 8053..8054: mod_base = OTHER; note = thymine
  modified_base 8066: mod_base = OTHER; note = thymine
  modified_base 8079: mod_base = OTHER; note = thymine
  modified_base 8091: mod_base = OTHER; note = thymine
  modified_base 8100: mod_base = OTHER; note = thymine
  modified_base 8111: mod_base = OTHER; note = thymine
  modified_base 8113..8114: mod_base = OTHER; note = thymine
  modified_base 8117: mod_base = OTHER; note = thymine
  modified_base 8126: mod_base = OTHER; note = thymine
  modified_base 8128..8130: mod_base = OTHER; note = thymine
  modified_base 8136: mod_base = OTHER; note = thymine
  modified_base 8140: mod_base = OTHER; note = thymine
  modified_base 8146: mod_base = OTHER; note = thymine
  modified_base 8150..8151: mod_base = OTHER; note = thymine
  modified_base 8162: mod_base = OTHER; note = thymine
  modified_base 8167..8168: mod_base = OTHER; note = thymine
  modified_base 8171: mod_base = OTHER; note = thymine
  modified_base 8176: mod_base = OTHER; note = thymine
  modified_base 8178: mod_base = OTHER; note = thymine
  modified_base 8181: mod_base = OTHER; note = thymine
  modified_base 8186..8187: mod_base = OTHER; note = thymine
  modified_base 8196: mod_base = OTHER; note = thymine
  modified_base 8198..8199: mod_base = OTHER; note = thymine
  modified_base 8202: mod_base = OTHER; note = thymine
  modified_base 8207..8208: mod_base = OTHER; note = thymine
  modified_base 8210: mod_base = OTHER; note = thymine
  modified_base 8222: mod_base = OTHER; note = thymine
  modified_base 8226..8227: mod_base = OTHER; note = thymine
  modified_base 8230..8232: mod_base = OTHER; note = thymine
  modified_base 8236..8238: mod_base = OTHER; note = thymine
  modified_base 8244..8245: mod_base = OTHER; note = thymine
  modified_base 8253: mod_base = OTHER; note = thymine
  modified_base 8256: mod_base = OTHER; note = thymine
  modified_base 8264: mod_base = OTHER; note = thymine
  modified_base 8268: mod_base = OTHER; note = thymine
  modified_base 8272..8273: mod_base = OTHER; note = thymine
  modified_base 8279..8281: mod_base = OTHER; note = thymine
  modified_base 8285: mod_base = OTHER; note = thymine
  modified_base 8287: mod_base = OTHER; note = thymine
  modified_base 8293..8294: mod_base = OTHER; note = thymine
  modified_base 8299: mod_base = OTHER; note = thymine
  modified_base 8301: mod_base = OTHER; note = thymine
  modified_base 8303: mod_base = OTHER; note = thymine
  modified_base 8314: mod_base = OTHER; note = thymine
  modified_base 8317: mod_base = OTHER; note = thymine
  modified_base 8320: mod_base = OTHER; note = thymine
  modified_base 8323: mod_base = OTHER; note = thymine
  modified_base 8326..8328: mod_base = OTHER; note = thymine
  modified_base 8331: mod_base = OTHER; note = thymine
  modified_base 8335: mod_base = OTHER; note = thymine
  modified_base 8340: mod_base = OTHER; note = thymine
  modified_base 8348: mod_base = OTHER; note = thymine
  modified_base 8350: mod_base = OTHER; note = thymine
  modified_base 8352: mod_base = OTHER; note = thymine
  modified_base 8355: mod_base = OTHER; note = thymine
  modified_base 8358: mod_base = OTHER; note = thymine
  modified_base 8360..8362: mod_base = OTHER; note = thymine
  modified_base 8365: mod_base = OTHER; note = thymine
  modified_base 8374..8376: mod_base = OTHER; note = thymine
  modified_base 8379: mod_base = OTHER; note = thymine
  modified_base 8383: mod_base = OTHER; note = thymine
  modified_base 8389: mod_base = OTHER; note = thymine
  modified_base 8393: mod_base = OTHER; note = thymine
  modified_base 8396: mod_base = OTHER; note = thymine
  modified_base 8400..8401: mod_base = OTHER; note = thymine
  modified_base 8409: mod_base = OTHER; note = thymine
  modified_base 8413..8414: mod_base = OTHER; note = thymine
  modified_base 8420: mod_base = OTHER; note = thymine
  modified_base 8426..8427: mod_base = OTHER; note = thymine
  modified_base 8429: mod_base = OTHER; note = thymine
  modified_base 8433: mod_base = OTHER; note = thymine
  modified_base 8436: mod_base = OTHER; note = thymine
  modified_base 8438: mod_base = OTHER; note = thymine
  modified_base 8440: mod_base = OTHER; note = thymine
  modified_base 8442: mod_base = OTHER; note = thymine
  modified_base 8446..8448: mod_base = OTHER; note = thymine
  modified_base 8455: mod_base = OTHER; note = thymine
  modified_base 8466..8467: mod_base = OTHER; note = thymine
  modified_base 8473: mod_base = OTHER; note = thymine
  modified_base 8477: mod_base = OTHER; note = thymine
  modified_base 8484: mod_base = OTHER; note = thymine
  modified_base 8487: mod_base = OTHER; note = thymine
  modified_base 8500..8502: mod_base = OTHER; note = thymine
  modified_base 8512..8513: mod_base = OTHER; note = thymine
  modified_base 8515: mod_base = OTHER; note = thymine
  modified_base 8517: mod_base = OTHER; note = thymine
  modified_base 8520..8521: mod_base = OTHER; note = thymine
  modified_base 8533..8535: mod_base = OTHER; note = thymine
  modified_base 8542: mod_base = OTHER; note = thymine
  modified_base 8546..8547: mod_base = OTHER; note = thymine
  modified_base 8559: mod_base = OTHER; note = thymine
  modified_base 8568..8570: mod_base = OTHER; note = thymine
  modified_base 8579: mod_base = OTHER; note = thymine
  modified_base 8583: mod_base = OTHER; note = thymine
  modified_base 8589: mod_base = OTHER; note = thymine
  modified_base 8595: mod_base = OTHER; note = thymine
  modified_base 8600: mod_base = OTHER; note = thymine
  modified_base 8603: mod_base = OTHER; note = thymine
  modified_base 8607: mod_base = OTHER; note = thymine
  modified_base 8609..8610: mod_base = OTHER; note = thymine
  modified_base 8619: mod_base = OTHER; note = thymine
  modified_base 8622: mod_base = OTHER; note = thymine
  modified_base 8632..8633: mod_base = OTHER; note = thymine
  modified_base 8640: mod_base = OTHER; note = thymine
  modified_base 8643..8644: mod_base = OTHER; note = thymine
  modified_base 8646..8647: mod_base = OTHER; note = thymine
  modified_base 8650: mod_base = OTHER; note = thymine
  modified_base 8655: mod_base = OTHER; note = thymine
  modified_base 8659: mod_base = OTHER; note = thymine
  modified_base 8675: mod_base = OTHER; note = thymine
  modified_base 8683..8685: mod_base = OTHER; note = thymine
  modified_base 8687..8688: mod_base = OTHER; note = thymine
  modified_base 8692..8693: mod_base = OTHER; note = thymine
  modified_base 8695: mod_base = OTHER; note = thymine
  modified_base 8704..8706: mod_base = OTHER; note = thymine
  modified_base 8711: mod_base = OTHER; note = thymine
  modified_base 8715: mod_base = OTHER; note = thymine
  modified_base 8722..8723: mod_base = OTHER; note = thymine
  modified_base 8726: mod_base = OTHER; note = thymine
  modified_base 8730: mod_base = OTHER; note = thymine
  modified_base 8739..8740: mod_base = OTHER; note = thymine
  modified_base 8744: mod_base = OTHER; note = thymine
  modified_base 8747..8748: mod_base = OTHER; note = thymine
  modified_base 8754..8755: mod_base = OTHER; note = thymine
  modified_base 8763..8764: mod_base = OTHER; note = thymine
  modified_base 8771: mod_base = OTHER; note = thymine
  modified_base 8773..8774: mod_base = OTHER; note = thymine
  modified_base 8777: mod_base = OTHER; note = thymine
  modified_base 8781: mod_base = OTHER; note = thymine
  modified_base 8784: mod_base = OTHER; note = thymine
  modified_base 8788: mod_base = OTHER; note = thymine
  modified_base 8793: mod_base = OTHER; note = thymine
  modified_base 8799..8801: mod_base = OTHER; note = thymine
  modified_base 8804: mod_base = OTHER; note = thymine
  modified_base 8808: mod_base = OTHER; note = thymine
  modified_base 8810: mod_base = OTHER; note = thymine
  modified_base 8812: mod_base = OTHER; note = thymine
  modified_base 8816: mod_base = OTHER; note = thymine
  modified_base 8822: mod_base = OTHER; note = thymine
  modified_base 8824: mod_base = OTHER; note = thymine
  modified_base 8833..8834: mod_base = OTHER; note = thymine
  modified_base 8837: mod_base = OTHER; note = thymine
  modified_base 8839: mod_base = OTHER; note = thymine
  modified_base 8849: mod_base = OTHER; note = thymine
  modified_base 8851: mod_base = OTHER; note = thymine
  modified_base 8855: mod_base = OTHER; note = thymine
  modified_base 8858: mod_base = OTHER; note = thymine
  modified_base 8862..8864: mod_base = OTHER; note = thymine
  modified_base 8867: mod_base = OTHER; note = thymine
  modified_base 8874..8875: mod_base = OTHER; note = thymine
  modified_base 8887: mod_base = OTHER; note = thymine
  modified_base 8891: mod_base = OTHER; note = thymine
  modified_base 8894: mod_base = OTHER; note = thymine
  modified_base 8903: mod_base = OTHER; note = thymine
  modified_base 8907: mod_base = OTHER; note = thymine
  modified_base 8909: mod_base = OTHER; note = thymine
  modified_base 8911: mod_base = OTHER; note = thymine
  modified_base 8913: mod_base = OTHER; note = thymine
  modified_base 8920: mod_base = OTHER; note = thymine
  modified_base 8924: mod_base = OTHER; note = thymine
  modified_base 8926..8928: mod_base = OTHER; note = thymine
  modified_base 8935: mod_base = OTHER; note = thymine
  modified_base 8937: mod_base = OTHER; note = thymine
  modified_base 8943: mod_base = OTHER; note = thymine
  modified_base 8954: mod_base = OTHER; note = thymine
  modified_base 8957..8958: mod_base = OTHER; note = thymine
  modified_base 8961: mod_base = OTHER; note = thymine
  modified_base 8966: mod_base = OTHER; note = thymine
  modified_base 8968..8969: mod_base = OTHER; note = thymine
  modified_base 8972: mod_base = OTHER; note = thymine
  modified_base 8978..8980: mod_base = OTHER; note = thymine
  modified_base 8988..8989: mod_base = OTHER; note = thymine
  modified_base 8991: mod_base = OTHER; note = thymine
  modified_base 8997: mod_base = OTHER; note = thymine
  modified_base 8999: mod_base = OTHER; note = thymine
  modified_base 9002: mod_base = OTHER; note = thymine
  modified_base 9010..9011: mod_base = OTHER; note = thymine
  modified_base 9015: mod_base = OTHER; note = thymine
  modified_base 9021..9022: mod_base = OTHER; note = thymine
  modified_base 9025: mod_base = OTHER; note = thymine
  modified_base 9032: mod_base = OTHER; note = thymine
  modified_base 9040: mod_base = OTHER; note = thymine
  modified_base 9051: mod_base = OTHER; note = thymine
  modified_base 9053: mod_base = OTHER; note = thymine
  modified_base 9057: mod_base = OTHER; note = thymine
  modified_base 9066: mod_base = OTHER; note = thymine
  modified_base 9072: mod_base = OTHER; note = thymine
  modified_base 9077..9078: mod_base = OTHER; note = thymine
  modified_base 9082: mod_base = OTHER; note = thymine
  modified_base 9084: mod_base = OTHER; note = thymine
  modified_base 9086..9087: mod_base = OTHER; note = thymine
  modified_base 9093: mod_base = OTHER; note = thymine
  modified_base 9095: mod_base = OTHER; note = thymine
  modified_base 9099: mod_base = OTHER; note = thymine
  modified_base 9104..9105: mod_base = OTHER; note = thymine
  modified_base 9108: mod_base = OTHER; note = thymine
  modified_base 9111: mod_base = OTHER; note = thymine
  modified_base 9121: mod_base = OTHER; note = thymine
  modified_base 9127: mod_base = OTHER; note = thymine
  modified_base 9129..9130: mod_base = OTHER; note = thymine
  modified_base 9141: mod_base = OTHER; note = thymine
  modified_base 9143..9144: mod_base = OTHER; note = thymine
  modified_base 9156..9157: mod_base = OTHER; note = thymine
  modified_base 9162: mod_base = OTHER; note = thymine
  modified_base 9168: mod_base = OTHER; note = thymine
  modified_base 9172..9173: mod_base = OTHER; note = thymine
  modified_base 9175..9176: mod_base = OTHER; note = thymine
  modified_base 9182..9183: mod_base = OTHER; note = thymine
  modified_base 9192: mod_base = OTHER; note = thymine
  modified_base 9199..9202: mod_base = OTHER; note = thymine
  modified_base 9207: mod_base = OTHER; note = thymine
  modified_base 9215: mod_base = OTHER; note = thymine
  modified_base 9218: mod_base = OTHER; note = thymine
  modified_base 9224..9225: mod_base = OTHER; note = thymine
  modified_base 9232..9233: mod_base = OTHER; note = thymine
  modified_base 9238: mod_base = OTHER; note = thymine
  modified_base 9248: mod_base = OTHER; note = thymine
  modified_base 9253: mod_base = OTHER; note = thymine
  modified_base 9257..9258: mod_base = OTHER; note = thymine
  modified_base 9268: mod_base = OTHER; note = thymine
  modified_base 9270: mod_base = OTHER; note = thymine
  modified_base 9272: mod_base = OTHER; note = thymine
  modified_base 9289: mod_base = OTHER; note = thymine
  modified_base 9291..9292: mod_base = OTHER; note = thymine
  modified_base 9303: mod_base = OTHER; note = thymine
  modified_base 9307: mod_base = OTHER; note = thymine
  modified_base 9310: mod_base = OTHER; note = thymine
  modified_base 9317..9318: mod_base = OTHER; note = thymine
  modified_base 9321: mod_base = OTHER; note = thymine
  modified_base 9328: mod_base = OTHER; note = thymine
  modified_base 9334..9335: mod_base = OTHER; note = thymine
  modified_base 9343: mod_base = OTHER; note = thymine
  modified_base 9348..9349: mod_base = OTHER; note = thymine
  modified_base 9356: mod_base = OTHER; note = thymine
  modified_base 9361: mod_base = OTHER; note = thymine
  modified_base 9368: mod_base = OTHER; note = thymine
  modified_base 9375: mod_base = OTHER; note = thymine
  modified_base 9378..9380: mod_base = OTHER; note = thymine
  modified_base 9386..9387: mod_base = OTHER; note = thymine
  modified_base 9391: mod_base = OTHER; note = thymine
  modified_base 9395: mod_base = OTHER; note = thymine
  modified_base 9397: mod_base = OTHER; note = thymine
  modified_base 9405: mod_base = OTHER; note = thymine
  modified_base 9409..9410: mod_base = OTHER; note = thymine
  modified_base 9414..9416: mod_base = OTHER; note = thymine
  modified_base 9418: mod_base = OTHER; note = thymine
  modified_base 9427..9428: mod_base = OTHER; note = thymine
  modified_base 9430: mod_base = OTHER; note = thymine
  modified_base 9437: mod_base = OTHER; note = thymine
  modified_base 9439: mod_base = OTHER; note = thymine
  modified_base 9441: mod_base = OTHER; note = thymine
  modified_base 9451: mod_base = OTHER; note = thymine
  modified_base 9453: mod_base = OTHER; note = thymine
  modified_base 9462: mod_base = OTHER; note = thymine
  modified_base 9465: mod_base = OTHER; note = thymine
  modified_base 9468..9470: mod_base = OTHER; note = thymine
  modified_base 9475: mod_base = OTHER; note = thymine
  modified_base 9478: mod_base = OTHER; note = thymine
  modified_base 9486..9487: mod_base = OTHER; note = thymine
  modified_base 9490: mod_base = OTHER; note = thymine
  modified_base 9497..9498: mod_base = OTHER; note = thymine
  modified_base 9503: mod_base = OTHER; note = thymine
  modified_base 9509: mod_base = OTHER; note = thymine
  modified_base 9520: mod_base = OTHER; note = thymine
  modified_base 9523: mod_base = OTHER; note = thymine
  modified_base 9528..9530: mod_base = OTHER; note = thymine
  modified_base 9534: mod_base = OTHER; note = thymine
  modified_base 9536..9537: mod_base = OTHER; note = thymine
  modified_base 9540: mod_base = OTHER; note = thymine
  modified_base 9544: mod_base = OTHER; note = thymine
  modified_base 9547: mod_base = OTHER; note = thymine
  modified_base 9556..9557: mod_base = OTHER; note = thymine
  modified_base 9562: mod_base = OTHER; note = thymine
  modified_base 9565..9566: mod_base = OTHER; note = thymine
  modified_base 9568: mod_base = OTHER; note = thymine
  modified_base 9574..9575: mod_base = OTHER; note = thymine
  modified_base 9587: mod_base = OTHER; note = thymine
  modified_base 9600: mod_base = OTHER; note = thymine
  modified_base 9612: mod_base = OTHER; note = thymine
  modified_base 9621: mod_base = OTHER; note = thymine
  modified_base 9632: mod_base = OTHER; note = thymine
  modified_base 9634..9635: mod_base = OTHER; note = thymine
  modified_base 9638: mod_base = OTHER; note = thymine
  modified_base 9647: mod_base = OTHER; note = thymine
  modified_base 9649..9651: mod_base = OTHER; note = thymine
  modified_base 9657: mod_base = OTHER; note = thymine
  modified_base 9661: mod_base = OTHER; note = thymine
  modified_base 9667: mod_base = OTHER; note = thymine
  modified_base 9671..9672: mod_base = OTHER; note = thymine
  modified_base 9683: mod_base = OTHER; note = thymine
  modified_base 9688..9689: mod_base = OTHER; note = thymine
  modified_base 9692: mod_base = OTHER; note = thymine
  modified_base 9697: mod_base = OTHER; note = thymine
  modified_base 9699: mod_base = OTHER; note = thymine
  modified_base 9702: mod_base = OTHER; note = thymine
  modified_base 9707..9708: mod_base = OTHER; note = thymine
  modified_base 9717: mod_base = OTHER; note = thymine
  modified_base 9719..9720: mod_base = OTHER; note = thymine
  modified_base 9723: mod_base = OTHER; note = thymine
  modified_base 9728..9729: mod_base = OTHER; note = thymine
  modified_base 9731: mod_base = OTHER; note = thymine
  modified_base 9743: mod_base = OTHER; note = thymine
  modified_base 9747..9748: mod_base = OTHER; note = thymine
  modified_base 9751..9753: mod_base = OTHER; note = thymine
  modified_base 9757..9759: mod_base = OTHER; note = thymine
  modified_base 9765..9766: mod_base = OTHER; note = thymine
  modified_base 9774: mod_base = OTHER; note = thymine
  modified_base 9777: mod_base = OTHER; note = thymine
  modified_base 9785: mod_base = OTHER; note = thymine
  modified_base 9789: mod_base = OTHER; note = thymine
  modified_base 9793..9794: mod_base = OTHER; note = thymine
  modified_base 9800..9802: mod_base = OTHER; note = thymine
  modified_base 9806: mod_base = OTHER; note = thymine
  modified_base 9808: mod_base = OTHER; note = thymine
  modified_base 9814..9815: mod_base = OTHER; note = thymine
  modified_base 9820: mod_base = OTHER; note = thymine
  modified_base 9822: mod_base = OTHER; note = thymine
  modified_base 9824: mod_base = OTHER; note = thymine
  modified_base 9835: mod_base = OTHER; note = thymine
  modified_base 9838: mod_base = OTHER; note = thymine
  modified_base 9841: mod_base = OTHER; note = thymine
  modified_base 9844: mod_base = OTHER; note = thymine
  modified_base 9847..9849: mod_base = OTHER; note = thymine
  modified_base 9852: mod_base = OTHER; note = thymine
  modified_base 9856: mod_base = OTHER; note = thymine
  modified_base 9861: mod_base = OTHER; note = thymine
  modified_base 9869: mod_base = OTHER; note = thymine
  modified_base 9871: mod_base = OTHER; note = thymine
  modified_base 9873: mod_base = OTHER; note = thymine
  modified_base 9876: mod_base = OTHER; note = thymine
  modified_base 9879: mod_base = OTHER; note = thymine
  modified_base 9881..9883: mod_base = OTHER; note = thymine
  modified_base 9886: mod_base = OTHER; note = thymine
  modified_base 9895..9897: mod_base = OTHER; note = thymine
  modified_base 9900: mod_base = OTHER; note = thymine
  modified_base 9904: mod_base = OTHER; note = thymine
  modified_base 9910: mod_base = OTHER; note = thymine
  modified_base 9914: mod_base = OTHER; note = thymine
  modified_base 9917: mod_base = OTHER; note = thymine
  modified_base 9921..9922: mod_base = OTHER; note = thymine
  modified_base 9930: mod_base = OTHER; note = thymine
  modified_base 9934..9935: mod_base = OTHER; note = thymine
  modified_base 9941: mod_base = OTHER; note = thymine
  modified_base 9947..9948: mod_base = OTHER; note = thymine
  modified_base 9950: mod_base = OTHER; note = thymine
  modified_base 9954: mod_base = OTHER; note = thymine
  modified_base 9957: mod_base = OTHER; note = thymine
  modified_base 9959: mod_base = OTHER; note = thymine
  modified_base 9961: mod_base = OTHER; note = thymine
  modified_base 9963: mod_base = OTHER; note = thymine
  modified_base 9967..9969: mod_base = OTHER; note = thymine
  modified_base 9976: mod_base = OTHER; note = thymine
  modified_base 9987..9988: mod_base = OTHER; note = thymine
  modified_base 9994: mod_base = OTHER; note = thymine
  modified_base 9998: mod_base = OTHER; note = thymine
  modified_base 10005: mod_base = OTHER; note = thymine
  modified_base 10008: mod_base = OTHER; note = thymine
  modified_base 10021..10023: mod_base = OTHER; note = thymine
  modified_base 10033..10034: mod_base = OTHER; note = thymine
  modified_base 10036: mod_base = OTHER; note = thymine
  modified_base 10038: mod_base = OTHER; note = thymine
  modified_base 10041..10042: mod_base = OTHER; note = thymine
  modified_base 10054..10056: mod_base = OTHER; note = thymine
  modified_base 10063: mod_base = OTHER; note = thymine
  modified_base 10067..10068: mod_base = OTHER; note = thymine
  modified_base 10080: mod_base = OTHER; note = thymine
  modified_base 10089..10091: mod_base = OTHER; note = thymine
  modified_base 10100: mod_base = OTHER; note = thymine
  modified_base 10104: mod_base = OTHER; note = thymine
  modified_base 10110: mod_base = OTHER; note = thymine
  modified_base 10116: mod_base = OTHER; note = thymine
  modified_base 10121: mod_base = OTHER; note = thymine
  modified_base 10124: mod_base = OTHER; note = thymine
  modified_base 10128: mod_base = OTHER; note = thymine
  modified_base 10130..10131: mod_base = OTHER; note = thymine
  modified_base 10140: mod_base = OTHER; note = thymine
  modified_base 10143: mod_base = OTHER; note = thymine
  modified_base 10153..10154: mod_base = OTHER; note = thymine
  modified_base 10161: mod_base = OTHER; note = thymine
  modified_base 10164..10165: mod_base = OTHER; note = thymine
  modified_base 10167..10168: mod_base = OTHER; note = thymine
  modified_base 10171: mod_base = OTHER; note = thymine
  modified_base 10176: mod_base = OTHER; note = thymine
  modified_base 10180: mod_base = OTHER; note = thymine
  modified_base 10196: mod_base = OTHER; note = thymine
  modified_base 10204..10206: mod_base = OTHER; note = thymine
  modified_base 10208..10209: mod_base = OTHER; note = thymine
  modified_base 10213..10214: mod_base = OTHER; note = thymine
  modified_base 10216: mod_base = OTHER; note = thymine
  modified_base 10225..10227: mod_base = OTHER; note = thymine
  modified_base 10232: mod_base = OTHER; note = thymine
  modified_base 10236: mod_base = OTHER; note = thymine
  modified_base 10243..10244: mod_base = OTHER; note = thymine
  modified_base 10247: mod_base = OTHER; note = thymine
  modified_base 10251: mod_base = OTHER; note = thymine
  modified_base 10260..10261: mod_base = OTHER; note = thymine
  modified_base 10265: mod_base = OTHER; note = thymine
  modified_base 10268..10269: mod_base = OTHER; note = thymine
  modified_base 10275: mod_base = OTHER; note = thymine
  modified_base 10277: mod_base = OTHER; note = thymine
  modified_base 10284..10285: mod_base = OTHER; note = thymine
  modified_base 10292: mod_base = OTHER; note = thymine
  modified_base 10294..10295: mod_base = OTHER; note = thymine
  modified_base 10298: mod_base = OTHER; note = thymine
  modified_base 10302: mod_base = OTHER; note = thymine
  modified_base 10305: mod_base = OTHER; note = thymine
  modified_base 10309: mod_base = OTHER; note = thymine
  modified_base 10314: mod_base = OTHER; note = thymine
  modified_base 10320..10322: mod_base = OTHER; note = thymine
  modified_base 10325: mod_base = OTHER; note = thymine
  modified_base 10329: mod_base = OTHER; note = thymine
  modified_base 10331: mod_base = OTHER; note = thymine
  modified_base 10333: mod_base = OTHER; note = thymine
  modified_base 10337: mod_base = OTHER; note = thymine
  modified_base 10343: mod_base = OTHER; note = thymine
  modified_base 10345: mod_base = OTHER; note = thymine
  modified_base 10354..10355: mod_base = OTHER; note = thymine
  modified_base 10358: mod_base = OTHER; note = thymine
  modified_base 10360: mod_base = OTHER; note = thymine
  modified_base 10370: mod_base = OTHER; note = thymine
  modified_base 10372: mod_base = OTHER; note = thymine
  modified_base 10376: mod_base = OTHER; note = thymine
  modified_base 10379: mod_base = OTHER; note = thymine
  modified_base 10383..10385: mod_base = OTHER; note = thymine
  modified_base 10388: mod_base = OTHER; note = thymine
  modified_base 10395..10396: mod_base = OTHER; note = thymine
  modified_base 10408: mod_base = OTHER; note = thymine
  modified_base 10412: mod_base = OTHER; note = thymine
  modified_base 10415: mod_base = OTHER; note = thymine
  modified_base 10424: mod_base = OTHER; note = thymine
  modified_base 10428: mod_base = OTHER; note = thymine
  modified_base 10430: mod_base = OTHER; note = thymine
  modified_base 10432: mod_base = OTHER; note = thymine
  modified_base 10434: mod_base = OTHER; note = thymine
  modified_base 10441: mod_base = OTHER; note = thymine
  modified_base 10445: mod_base = OTHER; note = thymine
  modified_base 10447..10449: mod_base = OTHER; note = thymine
  modified_base 10456: mod_base = OTHER; note = thymine
  modified_base 10458: mod_base = OTHER; note = thymine
  modified_base 10464: mod_base = OTHER; note = thymine
  modified_base 10471: mod_base = OTHER; note = thymine
  modified_base 10475: mod_base = OTHER; note = thymine
  modified_base 10478..10479: mod_base = OTHER; note = thymine
  modified_base 10482: mod_base = OTHER; note = thymine
  modified_base 10487: mod_base = OTHER; note = thymine
  modified_base 10489..10490: mod_base = OTHER; note = thymine
  modified_base 10493: mod_base = OTHER; note = thymine
  modified_base 10499..10501: mod_base = OTHER; note = thymine
  modified_base 10509..10510: mod_base = OTHER; note = thymine
  modified_base 10512: mod_base = OTHER; note = thymine
  modified_base 10518: mod_base = OTHER; note = thymine
  modified_base 10520: mod_base = OTHER; note = thymine
  modified_base 10523: mod_base = OTHER; note = thymine
  modified_base 10531..10532: mod_base = OTHER; note = thymine
  modified_base 10536: mod_base = OTHER; note = thymine
  modified_base 10542..10543: mod_base = OTHER; note = thymine
  modified_base 10546: mod_base = OTHER; note = thymine
  modified_base 10553: mod_base = OTHER; note = thymine
  modified_base 10561: mod_base = OTHER; note = thymine
  modified_base 10572: mod_base = OTHER; note = thymine
  modified_base 10574..10575: mod_base = OTHER; note = thymine
  modified_base 10578: mod_base = OTHER; note = thymine
  modified_base 10582: mod_base = OTHER; note = thymine
  modified_base 10584: mod_base = OTHER; note = thymine
  modified_base 10587: mod_base = OTHER; note = thymine
  modified_base 10593: mod_base = OTHER; note = thymine
  modified_base 10598..10599: mod_base = OTHER; note = thymine
  modified_base 10603: mod_base = OTHER; note = thymine
  modified_base 10605: mod_base = OTHER; note = thymine
  modified_base 10607..10608: mod_base = OTHER; note = thymine
  modified_base 10614: mod_base = OTHER; note = thymine
  modified_base 10616: mod_base = OTHER; note = thymine
  modified_base 10620: mod_base = OTHER; note = thymine
  modified_base 10625..10626: mod_base = OTHER; note = thymine
  modified_base 10629: mod_base = OTHER; note = thymine
  modified_base 10632: mod_base = OTHER; note = thymine
  modified_base 10642: mod_base = OTHER; note = thymine
  modified_base 10648: mod_base = OTHER; note = thymine
  modified_base 10650..10651: mod_base = OTHER; note = thymine
aaaggaggtgttccagccgcaccttccggtacggctaccttgttacgacttagccccagtcaccagttttaccctaggccgatcctcgcggttacggacttcaggtacccccggctcccatggcttgacgggcggtgtgtacaaggcccgggaacgtattcaccgcgccatggctgatgcgcgattactagcgaatccagcttcacggagtcgggttgcagactccgatccgaactgagacgtggtttggggattcgctccctgtcgccaggtggcctccctttgtccacgccattgtaacacgtgtgtcgccccggatgtaagggccgtgctgatttgacgtcatccccgccttcctcgcagcttacgctggcagtcccaccagagtcctcagctttacctgttagtaactagtggcaagggttgcgctcgttatggcacttaagccgacacctcacggcacgagctgacgacaaccatgcagcacctcgcaaacggctattgctagaaaaggtgtttccacctcggtccgaatgcgttcaaacccgggtaaggttcctcgcgtatcatcgaattaaaccacatgttcctccgcttgtgcgggcccccgtcaattcctttgagtttcaccgttgccggcgtactccccaggtggatcacttaacgctttcgctgtgccgcttacagtgtatcgcaaacagctagtgatcatcgtttactgcgtggactaccagggtatctaatcctgtttgatccccacgctttcgtgcatcagcgtcagtaatggcttggcaggctgccttcgcaatcggggttctgcgtgatatctatgcatttcaccgctacaccacgcattccgcctgcctcaaacatactcaagccccccagtttcaacggcaattctatggttgagccacagactttcaccgctgacttaaaaggccgcctacgcaccctttaaacccaataaatccggataacgctcggatcctccgtattaccgcggctgctggcacggagttagccgatccttattcataaggtacatacaaaacgggacacgtcccgcactttattcccttataaaagaggtttacgatccatagaaccttcatccctcacgcgacttggctggttcaggcttacgcccattgaccaatattcctcactgctgcctcccgtaggagtttggtccgtgtctcagtaccaatgtgggggaccttcctctcagaacccctatccatcgtcggtttggtgggccgttaccccgccaactgcctaatggaacgcatgcctatctatcagcgatgaatctttagcaaatatccccatgcggggcccctgcttcatgcggtattagtccgactttcgccgggttatccccctctgataggcaagttgcatacgcgttactcacccgtgcgccggtcgccggcgggtatcgctacccccgctgcccctcgacttgcatgtgttaagcctgtcgctagcgttcatcctgagccaggatcaaactcttcgaaaggaggtgttccagccgcaccttccggtacggctaccttgttacgacttagccccagtcaccagttttaccctaggccgatcctttcggttacggacttcaggtacccccggctcccatggcttgacgggcggtgtgtacaaggcccgggaacgtattcaccgcgccatggctgatgcgcgattactagcgaatccagcttcacggagtcgggttgcagactccgatccgaactgagacgtggtttggggattcgctccctgtcgccaggtggcctccctttgtccacgccattgtaacacgtgtgtcgccccggatgtaagggccgtgctgatttgacgtcatccccgccttcctcgcagcttacgctggcagtcccaccagagtcctcagctttacctgttagtaactagtggcaagggttgcgctcgttatggcacttaagccgacacctcacggcacgagctgacgacaaccatgcagcacctcgcaaacggctattgctagaaaaggtgtttccacctcggtccgaatgcgttcaaacccgggtaaggttcctcgcgtatcatcgaattaaaccacatgttcctccgcttgtgcgggcccccgtcaattcctttgagtttcaccgttgccggcgtactccccaggtggatcacttaacgctttcgctgtgccgcttacagtgtatcgcaaacagctagtgatcatcgtttactgcgtggactaccagggtatctaatcctgtttgatccccacgctttcgtgcatcagcgtcagtaatggcttggcaggctgccttcgcaatcggggttctgcgtgatatctatgcatttcaccgctacaccacgcattccgcctgcctcaaacatactcaagccccccagtttcaacggcaattctatggttgagccacagactttcaccgctgacttaaaaggccgcctacgcaccctttaaacccaataaatccggataacgctcggatcctccgtattaccgcggctgctggcacggagttagccgatccttattcataaggtacatacaaaacgggacacgtcccgcactttattcccttataaaagaggtttacgatccatagaaccttcatccctcacgcgacttggctggttcagcctctcggccattgaccaatattcctcactgctgcctcccgtaggagtttggtccgtgtctcagtaccaatgtgggggaccttcctctcagaacccctatccatcgtcggtttggtgggccgttaccccgccaactgcctaatggaacgcatgcctatctatcagcgatgaatctttagcaaatatccccatgcggggcccctgcttcatgcggtattagtccgactttcgccgggttatccccctctgataggtaagttgcatacgcgttactcacccgtgcgccggtcgccggcgggtattgctacccccgctgcccctcgacttgcatgtgttaagcctgtcgctagcgttcatcctgagccaggatcaaactcttcgcgaagagtttgatcctggctcaggatgaacgctagcgacaggcttaacacatgcaagtcgaggggcagcacaggtagcaataccgggtggcgaccggcgcacgggtgagtaacgcgtatgcaacttacctatcagagggggataacccggcgaaagtcggactaataccgcatgaagcaggggtcccgcatggggatatttgctaaagattcatcgctgatagataggcatgcgttccattaggcagttggcggggtaacggcccaccaaaccgacgatggataggggttctgagaggaaggtcccccacattggtactgagacacggaccaaactcctacgggaggcagcagtgaggaatattggtcaatggccgagaggctgaaccagccaagtcgcgtgagggatgaaggttctatggatcgtaaacctcttttataagggaataaagtgcgggacgtgtcccgttttgtatgtaccttatgaataaggatcggctaactccgtgccagcagccgcggtaatacggaggatccgagcgttatccggatttattgggtttaaagggtgcgtaggcggccttttaagtcagcggtgaaagtctgtggctcaaccatagaattgccgttgaaactggggggcttgagtatgtttgaggcaggcggaatgcgtggtgtagcggtgaaatgcatagatatcacgcagaaccccgattgcgaaggcagcctgccaagccattactgacgctgatgcacgaaagcgtggggatcaaacaggattagataccctggtagtccacgcagtaaacgatgatcactagctgtttgcgatacactgtaagcggcacagcgaaagcgttaagtgatccacctggggagtacgccggcaacggtgaaactcaaaggaattgacgggggcccgcacaagcggaggaacatgtggtttaattcgatgatacgcgaggaaccttacccgggtttgaacgcattcggaccgaggtggaaacaccttttctagcaatagccgtttgcgaggtgctgcatggttgtcgtcagctcgtgccgtgaggtgtcggcttaagtgccataacgagcgcaacccttgccactagttactaacaggtaaagctgaggactctggtgggactgccagcgtaagctgcgaggaaggcggggatgacgtcaaatcagcacggcccttacatccggggcgacacacgtgttacaatggcgtggacaaagggaagccacctggcgacagggagcgaatccccaaaccacgtctcagttcggatcggagtctgcaacccgactccgtgaagctggattcgctagtaatcgcgcatcagccatggcgcggtgaatacgttcccgggccttgtacacaccgcccgtcaagccatgggagccgggggtacctgaagtccgtaactgcgaggatcggcctagggtaaaactggtgactggggctaagtcgtaacaaggtagccgtaccggaaggtgcggctggaacacctcctttcgaagagtttgatcctggctcaggatgaacgctagcgacaggcttaacacatgcaagtcgaggggcagcacaggtagcaataccgggtggcgaccggcgcacgggtgagtaacgcgtatgcaacttacctatcagagggggataacccggcgaaagtcggactaataccgcatgaagcaggggtcccgcatggggatatttgctaaagattcatcgctgatagataggcatgcgttccattaggcagttggcggggtaacggcccaccaaaccgacgatggataggggttctgagaggaaggtcccccacattggtactgagacacggaccaaactcctacgggaggcagcagtgaggaatattggtcaatggccgagaggctgaaccagccaagtcgcgtgagggatgaaggttctatggatcgtaaacctcttttataagggaataaagtgcgggacgtgtcccgttttgtatgtaccttatgaataaggatcggctaactccgtgccagcagccgcggtaatacggaggatccgagcgttatccggatttattgggtttaaagggtgcgtaggcggccttttaagtcagcggtgaaagtctgtggctcaaccatagaattgccgttgaaactggggggcttgagtatgtttgaggcaggcggaatgcgtggtgtagcggtgaaatgcatagatatcacgcagaaccccgattgcgaaggcagcctgccaagccattactgacgctgatgcacgaaagcgtggggatcaaacaggattagataccctggtagtccacgcagtaaacgatgatcactagctgtttgcgatacactgtaagcggcacagcgaaagcgttaagtgatccacctggggagtacgccggcaacggtgaaactcaaaggaattgacgggggcccgcacaagcggaggaacatgtggtttaattcgatgatacgcgaggaaccttacccgggtttgaacgcattcggaccgaggtggaaacaccttttctagcaatagccgtttgcgaggtgctgcatggttgtcgtcagctcgtgccgtgaggtgtcggcttaagtgccataacgagcgcaacccttgccactagttactaacaggtaaagctgaggactctggtgggactgccagcgtaagctgcgaggaaggcggggatgacgtcaaatcagcacggcccttacatccggggcgacacacgtgttacaatggcgtggacaaagggaagccacctggcgacagggagcgaatccccaaaccacgtctcagttcggatcggagtctgcaacccgactccgtgaagctggattcgctagtaatcgcgcatcagccatggcgcggtgaatacgttcccgggccttgtacacaccgcccgtcaagccatgggagccgggggtacctgaagtccgtaaccgcgaggatcggcctagggtaaaactggtgactggggctaagtcgtaacaaggtagccgtaccggaaggtgcggctggaacacctcctttcgaagagtttgatcctggctcaggatgaacgctagcgacaggcttaacacatgcaagtcgaggggcagcacaggtagcaataccgggtggcgaccggcgcacgggtgagtaacgcgtatgcaacttacctatcagagggggataacccggcgaaagtcggactaataccgcatgaagcaggggtcccgcatggggatatttgctaaagattcatcgctgatagataggcatgcgttccattaggcagttggcggggtaacggcccaccaaaccgacgatggatagggggcctagttctgagaggaaggtcccccacattggtactgagacacggaccaaactcctacgggaggcagcagtgaggaatattggtcaatggccgagaggctgaaccagccaagtcgcgtgagggatgaaggttctatggatcgtaaacctcttttataagggaataaagtgcgggacgtgtcccgttttgtatgtaccttatgaataaggatcggctaactccgtgccagcagccgcggtaatacggaggatccgagcgttatccggatttattgggtttaaagggtgcgtaggcggccttttaagtcagcggtgaaagtctgtggctcaaccatagaattgccgttgaaactggggggcttgagtatgtttgaggcaggcggaatgcgtggtgtagcggtgaaatgcatagatatcacgcagaaccccgattgcgaaggcagcctgccaagccattactgacgctgatgcacgaaagcgtggggatcaaacaggattagataccctggtagtccacgcagtaaacgatgatcactagctgtttgcgatacactgtaagcggcacagcgaaagcgttaagtgatccacctggggagtacgccggcaacggtgaaactcaaaggaattgacgggggcccgcacaagcggaggaacatgtggtttaattcgatgatacgcgaggaaccttacccgggtttgaacgcattcggaccgaggtggaaacaccttttctagcaatagccgtttgcgaggtgctgcatggttgtcgtcagctcgtgccgtgaggtgtcggcttaagtgccataacgagcgcaacccttgccactagttactaacaggtaaagctgaggactctggtgggactgccagcgtaagctgcgaggaaggcggggatgacgtcaaatcagcacggcccttacatccggggcgacacacgtgttacaatggcgtggacaaagggaagccacctggcgacagggagcgaatccccaaaccacgtctcagttcggatcggagtctgcaacccgactccgtgaagctggattcgctagtaatcgcgcatcagccatggcgcggtgaatacgttcccgggccttgtacacaccgcccgtcaagccatgggagccgggggtacctgaagtccgtaactgcgaggatcggcctagggtaaaactggtgactggggctaagtcgtaacaaggtagccgtaccggaaggtgcggctggaacacctcctttaaaggaggtgttccagccgcaccttccggtacggctaccttgttacgacttagccccagtcaccagttttaccctaggccgatcctttcggttacggacttcaggtacccccggctcccatggcttgacgggcggtgtgtacaaggcccgggaacgtattcaccgcgccatggctgatgcgcgattactagcgaatccagcttcacggagtcgggttgcagactccgatccgaactgagacgtggtttggggattcgctccctgtcgccaggtggcctccctttgtccacgccattgtaacacgtgtgtcgccccggatgtaagggccgtgctgatttgacgtcatccccgccttcctcgcagcttacgctggcagtcccaccagagtcctcagctttacctgttagtaactagtggcaagggttgcgctcgttatggcacttaagccgacacctcacggcacgagctgacgacaaccatgcagcacctcgcaaacggctattgctagaaaaggtgtttccacctcggtccgaatgcgttcaaacccgggtaaggttcctcgcgtatcatcgaattaaaccacatgttcctccgcttgtgcgggcccccgtcaattcctttgagtttcaccgttgccggcgtactccccaggtggatcacttaacgctttcgctgtgccgcttacagtgtatcgcaaacagctagtgatcatcgtttactgcgtggactaccagggtatctaatcctgtttgatccccacgctttcgtgcatcagcgtcagtaatggcttggcaggctgccttcgcaatcggggttctgcgtgatatctatgcatttcaccgctacaccacgcattccgcctgcctcaaacatactcaagccccccagtttcaacggcaattctatggttgagccacagactttcaccgctgacttaaaaggccgcctacgcaccctttaaacccaataaatccggataacgctcggatcctccgtattaccgcggctgctggcacggagttagccgatccttattcataaggtacatacaaaacgggacacgtcccgcactttattcccttataaaagaggtttacgatccatagaaccttcatccctcacgcgacttggctggttcaggcttacgcccattgaccaatattcctcactgctgcctcccgtaggagtttggtccgtgtctcagtaccaatgtgggggaccttcctctcagaacccctatccatcgtcggtttggtgggccgttaccccgccaactgcctaatggaacgcatgcctatctatcagcgatgaatctttagcaaatatccccatgcggggcccctgcttcatgcggtattagtccgactttcgccgggttatccccctctgataggcaagttgcatacgcgttactcacccgtgcgccggtcgccggcgggtatcgctacccccgctgcccctcgacttgcatgtgttaagcctgtcgctagcgttcatcctgagccaggatcaaactcttcgaaaggaggtgttccagccgcaccttccggtacggctaccttgttacgacttagccccagtcaccagttttaccctaggccgatcctcgcggttacggacttcaggtacccccggctcccatggcttgacgggcggtgtgtacaaggcccgggaacgtattcaccgcgccatggctgatgcgcgattactagcgaatccagcttcacggagtcgggttgcagactccgatccgaactgagacgtggtttggggattcgctccctgtcgccaggtggcttccctttgtccacgccattgtaacacgtgtgtcgccccggatgtaagggccgtgctgatttgacgtcatccccgccttcctcgcagcttacgctggcagtcccaccagagtcctcagctttacctgttagtaactagtggcaagggttgcgctcgttatggcacttaagccgacacctcacggcacgagctgacgacaaccatgcagcacctcgcaaacggctattgctagaaaaggtgtttccacctcggtccgaatgcgttcaaacccgggtaaggttcctcgcgtatcatcgaattaaaccacatgttcctccgcttgtgcgggcccccgtcaattcctttgagtttcaccgttgccggcgtactccccaggtggatcacttaacgctttcgctgtgccgcttacagtgtatcgcaaacagctagtgatcatcgtttactgcgtggactaccagggtatctaatcctgtttgatccccacgctttcgtgcatcagcgtcagtaatggcttggcaggctgccttcgcaatcggggttctgcgtgatatctatgcatttcaccgctacaccacgcattccgcctgcctcaaacatactcaagccccccagtttcaacggcaattctatggttgagccacagactttcaccgctgacttaaaaggccgcctacgcaccctttaaacccaataaatccggataacgctcggatcctccgtattaccgcggctgctggcacggagttagccgatccttattcataaggtacatacaaaacgggacacgtcccgcactttattcccttataaaagaggtttacgatccatagaaccttcatccctcacgcgacttggctggttcagcctctcggccattgaccaatattcctcactgctgcctcccgtaggagtttggtccgtgtctcagtaccaatgtgggggaccttcctctcagaacccctatccatcgtcggtttggtgggccgttaccccgccaactgcctaatggaacgcatgcctatctatcagcgatgaatctttagcaaatatccccatgcgggatccctgcttcatgcggtattagtccgactttcgccgggttatccccctctgataggcaagttgcatacgcgttactcacccgtgcgccggtcgccacccggtattgctacctgtgctgcccctcgacttgcatgtgttaagcctgtcgctagcgttcatcctgagccaggatcaaactcttcg

(19) United States
(12) Patent Application Publication
(10) Pub. No.: US 2025/0327021 A1
(43) Pub. Date: Oct. 23, 2025

(54) PARABACTEROIDES DISTASONIS STRAIN ISOLATED FROM HUMAN FECES AND APPLICATIONS THEREOF

(71) Applicant: GREENTECH, SAINT-BEAUZIRE (FR)

(72) Inventors: Sandie GERVASON, Pont-du-chateau (FR); Edith FILAIRE, Mirefleurs (FR); Jean-Yves BERTHON, Romagnat (FR); Frederic CARVALHO, Clermont-Ferrand (FR)

(73) Assignee: GREENTECH, SAINT-BEAUZIRE (FR)

(21) Appl. No.: 18/851,432

(22) PCT Filed: Mar. 31, 2023
(86) PCT No.: PCT/EP2023/058580

§ 371 Date: Sep. 26, 2024

(30) Foreign Application Priority Data
Apr. 1, 2022 (EP) 22166365.1

(51) Int. Cl. C12N 1/20; A61K 35/00; A61P 1/00; A61P 29/00; C12R 1/01

(52) U.S. Cl. CPC C12N 1/205; A61P 1/00; A61P 29/00; A61K 2035/115; C12R 2001/01

(57) ABSTRACT

The present invention concerns a Parabacteroides distasonis strain deposited under accession number CNCM I-5828. This strain can be useful in multiple applications, in particular incorporated in a nutritional supplement or in a composition for use as a medicament, especially in the prevention or treatment of visceral pain, preferably induced by Inflammatory Bowel Disease including Crohn's disease and Ulcerative Colitis, or by Irritable Bowel Syndrome.

The present invention concerns a Parabacteroides distasonis strain deposited under accession number CNCM I-5828. This strain can be useful in multiple applications, in particular incorporated in a nutritional supplement or in a composition for use as a medicament, especially in the prevention or treatment of visceral pain, particularly observed in Inflammatory Bowel Disease including Crohn's disease and Ulcerative Colitis, or in Irritable Bowel Syndrome.

### TECHNICAL FIELD

This invention is in the field of bacterial strains isolated from human, especially human feces, and compositions including this strain. In preferred embodiments, the invention is in the field of nutritional supplement composition and composition for use as a medicament including this strain. This composition for use as a medicament is especially for preventing or treating visceral pain.

### BACKGROUND ART

The microbiome comprises several types of microorganisms (bacteria, archaea, viruses, parasites and fungi), their genomes, and their surrounding environment, including the gastrointestinal tract, oral mucosa, urogenital and respiratory systems, and the skin surface [1]. Bacteria are the group of microorganisms that compose the microbiome which is most studied [2]. It is estimated that their number in the entire human body is of the same order as the number of human cells [3]. Many bacteria reside in the intestines [4]. Due to their huge number and diversity, they can significantly affect normal physiology and modify the host's susceptibility to diseases [5]. Indeed, changes in microbiota composition have been documented in Inflammatory Bowel Disease (IBD) and in Irritable Bowel Syndrome (IBS).

Crohn's disease (CD) and ulcerative colitis (UC) are part of IBD and are characterized by inflammation of epithelial barrier of intestinal tract. [6]. The pattern of dysbiosis mostly associated with IBD patients is a decrease in commensal bacteria diversity, and a relative increase of bacterial species belonging to Enterobacteriaceae [7]. While data are heterogeneous, the gut microbiota appears to play an important role in IBD pathogenesis. A recently published study conducted on 132 IBD patients provided the most comprehensive description to date of host and microbial activities in IBD, demonstrating that the gut microbiome, the molecular functional profile and the host immune factors are key component in IBD [8]. Beside to identify microbial, transcriptomic and metabolomic profiles, recent studies highlighted metabolites signature in patients suffered from IBD [9]. Similarly, another recently published study showed a disruption in the taxa of the Lachnospiraceae and Ruminococcaceae families in IBD patients compared to controls, and demonstrated that disturbance in the distinct networks of taxa associations is involved in CD and UC disease development [8].

In recognition of the potential positive effect that several bacterial strains may have on the animal gut, various strains have been proposed as new therapeutic strategies in various diseases. Several strains, including mostly Lactobacillus and Bifidobacterium strains, have also been proposed for treatment of inflammatory and autoimmune diseases that are not directly linked to the gastrointestinal tract. However, the relationship between different diseases and different bacterial strains, and precise effects of bacterial strains on the gut and at a systemic level and on any types of diseases are poorly characterized.

IBS or spastic colon is a disorder characterized by abdominal pain and changes in bowel habits, which are not associated with any abnormalities seen on routine clinical diagnostic. It is common and makes up 20-50% of visits to gastroenterologists. Chronic pain within the digestive tract remains a major unmet medical problem. It accompanies common digestive diseases, is poorly understood and is difficult to treat.

Lower abdominal pain, and bloating associated with alteration of bowel habits and abdominal discomfort relieved with defecation are the most frequent symptoms. It must be understood for the good comprehension of the present invention that IBS is a syndrome and that under this expression are collected several symptoms observed on patients suffering from the gastro-intestinal tract. Thus, therapeutic solutions aim to treat or to improve several symptoms associated to IBS.

Visceral pain is a common symptom observed in Inflammatory Bowel Disease (IBD: Crohn's disease, ulcerative colitis). Pain is an important manifestation of inflammation, as inflammatory cytokines and mediators sensitize primary afferent neurons. It should thus not be surprising that pain is one of the presenting symptoms in about 50%-70% of patients experiencing the initial onset or exacerbations of IBD [10]. The current treatment options are mostly focusing on the reduction of inflammation for IBD or the intestinal motility disturbances for IBS. Only a few therapies aim directly at diminishing abdominal pain [11]. Today, only symptomatic treatments are proposed and alternatives solutions are currently explored to relieve visceral and abdominal pain in patients suffering from IBD or IBS. Among, them, adapted diet, fecal microbiota transplantation or use of “biotics” products (including probiotics, prebiotics, synbiotics, metabiotics or parabiotics) are explored strategies to restore microbiota homeostasis and to limit inflammation.

At the intestinal level, the stimuli of pain can be varied, both mechanical (intestinal peristalsis, distension, stretching), or chemical (neurotransmitters or inflammation mediators for example) and can come from various origins: both intestinal cells, immunity cells or the intestinal microbiota. There are many receptors involved in pain signaling. Nociceptive stimuli, whether mechanical, chemical, or thermal, are picked up by nociceptors called primary afferent neurons and carry information to the posterior horn of the spinal cord. In pain perception process, transmission of sensorial information from the intestine to the brain is ensured by extrinsic nervous system whose cell bodies localized in dorsal root ganglia (DRG) playing a crucial function and relaying nociceptive information to the brain. Spinal afferences play a crucial role in the transmission of information about physiological disorders, causing bloating, discomfort or visceral pain. As a true sentinel system, extrinsic innervation ensures communication between the intestine and the brain, transmitting digestive and physiological information to the central nervous system where it will be treated. This is the pathway that nerve impulses use to get to the central nervous system. Pain perception involves complex cellular and molecular mechanisms in several stages. In the first step, intestinal information is encoded and relayed to the posterior horn of the spinal cord. This is the transduction step. During transduction, the nociceptive stimulus depolarizes the free termination of nociceptors, thereby activating the opening of voltage-dependent sodium channels and causing the generation of an action potential (PA). This nerve impulse induces the massive entry of positively charged ions at the central terminus in the posterior horn of the spinal cord and is transmitted to a secondary neuron up to the thalamus where it will synapse with a third neuron. This is the transmission phase in the brain. The latter then directs the nociceptive information to different regions of the somatosensory cortex, capable of distinguishing sensory information from affective or emotional information.

Animal models of colitis have been studied to develop alternative treatment options for patients with visceral pain, by measuring colonic hypersensitivity. In addition to exhibiting responses to inflammation that are similar to those seen in humans, rodent models have allowed the identification of unique characteristics of populations of sensory neurons that innervate the gut. Interestingly, there are common features of sensory neurons innervating different organs including colon, stomach, and pancreas. All these structures are densely innervated by sensory fibers that express calcitonin gene-related peptide (CGRP). This peptide, a potent vasodilator, is released by peripheral sensory nerve terminals in response to noxious and non-noxious stimulation and can have effects on vasculature and other structures. This property highlights one of the underappreciated aspects of visceral (and most somatic) sensory neurons; ie, in addition to detecting internal (visceral) or external (environmental) stimuli, sensory neurons release a number of compounds including CGRP and substance P (SP) that can have peripheral motor and/or autocrine/paracrine effects on other cells or sensory neurons. Under normal conditions, these compounds contribute to homeostatic regulation, but in disease states, including those with an inflammatory component, they may exacerbate symptoms (“neurogenic inflammation”). Most TRPV1-expressing neurons release SP and/or CGRP and it is thought that the release of these compounds may drive inflammatory responses.

IBD and IBS are characterized, among other things, by the development of visceral hypersensitivity. Several mediators may be at the origin such as serotonin, histamine, bradykinin, substance P, ATP, prostaglandins as well as all mediators of inflammation such as cytokines and chemokines. These molecules interact with neuronal receptors involved in the signaling of visceral pain. Activation of these receptors can lead to an anti-nociceptive or, on the contrary, pro-nociceptive effect depending on whether they are activators or inhibitors and participate to pain modulation. Several receptors such as serotoninergic, histaminergic, bradykinin receptors, or receptors belonging to the TRP (Transient receptor potential) family such as TRPV1, TRPV4, Transient receptor potential ankyrin 1 (TRPA1), but also purinergic or tachykinin receptors involvement in modulation of visceral painful process have been studied and quickly became therapeutic targets. One of them, TRPV1, may play a central role in setting the overall sensitivity of colonic afferences. In fact, colonic hypersensitivity developed in response to inflammatory mediators in wild-type mice is absent in TRPV1-deficient mice. In addition, TRPV1 is overexpressed and correlated with pain severity in IBD [13] and in IBS [14].

Moreover, pharmacologic inhibition of the TRPV1 blocks the development of hyperalgesia during colitis [15]. Whereas TRPV1 has received considerable attention recently, no single channel is likely to be responsible for colonic hypersensitivity. Deletion of a member of acid sensitive ion channels (ASIC3) or another member of the TRP (transient receptor potential) family (TRPV4) blunt or completely block the development of hyperalgesia in response to colonic inflammation [15]. TRPA1 has also been reported to contribute to visceral pain-like behavior in dextran sulfate sodium (DSS)-evoked colitis. Pain process involves complex molecular pathways and various neuronal receptors that can, together, contribute to generation of pain. This is the case of G-coupled protein receptor (GPCR) whose activation can sensitize nociceptors belonging to TRP family. This is the reason why GPCR has become therapeutic target for the treatment of chronic pain in the digestive system [16].

Parabacteroides distasonis is a bacterium belonging to Porphyromonadaceae family and to Parabacteroides genus. Initially classified in the Bacteroides genus based on its phenotypic and morphological characteristics, this bacterial species was recently reclassified and belongs to the Tannerellaceae including Parabacteroides and Tannerella genera. Indeed, beyond its phenotypic characteristics, this species proves to be closer phylogenetically to the species Tannerella fosythensis. The genus Parabacteroides currently counts 15 species: Parabacteroides acidifaciens, Parabacteroides bouchesdurhonensis, Parabacteroides chartae, Parabacteroides chinchillae, Parabacteroides chongii, Parabacteroides distasonis, Parabacteroides faecis, Parabacteroides goldsteinii, Parabacteroides gordonii, Parabacteroides johnsonii, Parabacteroides massiliensis, Parabacteroides merdae, Parabacteroides pacaensis, Parabacteroides provencensis, Parabacteroides timonensis. Parabacteroides distasonis represents the reference species and the strain P. distasonis ATCC 8503 (DSM20701) is the reference strain to the genus Parabacteroides to have been reclassified [17].

Parabacteroides distasonis is the type of strain for the genus Parabacteroides, a group of gram-negative anaerobic bacteria that commonly colonize the gastrointestinal tract of numerous species. Anti-inflammatory properties of P. distasonis have been demonstrated [18]. In addition to exert anti-inflammatory properties, some P. distasonis strains have demonstrated beneficial effect on strengthening of epithelial barrier.

For the above reasons, there is a requirement for new ways of treating visceral pain, in particular induced by IBS or IBD. Moreover, there is a constant need to identify new bacterial strains which can be useful in many technical domains, and especially in nutritional supplements and as medicaments.

Surprisingly, the Applicant identified from human feces a new strain of the genus Parabacteroides, from the species Parabacteroides distasonis, which demonstrates an unexpected and strong efficacy to prevent or treat visceral pain.

### DISCLOSURE OF THE INVENTION

Therefore, in a first aspect, the invention concerns a Parabacteroides distasonis strain deposited under accession number CNCM I-5828.

The Parabacteroides distasonis strain according to the invention has a 16s rRNA gene sequence as shown in SEQ ID NO:1.

The Parabacteroides distasonis strain according to the invention has been isolated from healthy human feces.

The bacterial strain according to the invention has been named “P. distasonis F1-2”. This name will be used especially in the examples to describe the strain according to the invention and deposited under accession number CNCM I-5828, and having a 16s rRNA gene sequence as shown in SEQ ID NO:1.

The bacterial strain used in the invention has been characterized in particular by sequencing 16s rRNA gene. The 16s rRNA gene sequence (SEQ ID NO:1) of this bacterial strain is at least 99.61% identical to the 16s RNA sequence of the species Parabacteroides distasonis (Parabacteroides distasonis ATCC 8503). 16s rRNA gene sequencing is commonly used as a tool to identify bacteria at the species level and assist with differentiating between closely related bacterial species. The sequence of the complete genome of Parabacteroides distasonis ATCC 8503 is known in the art, and can be found on the following link: https://www.ebi.ac.uk/ena/browser/api/fasta/CP000140.1?lineLimit=1000

The bacterial strain of the invention has been isolated from healthy male human.

In a second aspect, the invention concerns a composition comprising a Parabacteroides distasonis strain deposited under accession number CNCM I-5828.

In an embodiment, said composition can further comprises any type of other compounds which could be suitable for the purpose of the composition.

In a third aspect, the invention concerns a nutritional supplement composition comprising a bacterial strain according to the invention.

By “nutritional supplement”, “Food product”, “Food supplement”, “nutraceutical composition” or “Dietary supplement” is identically intended according to the invention a product that may provide nutritional benefit. This supplement can be taken alone or be formulated with other compounds in order to make the composition more attractive to consume by being more similar to a common food item. This supplement can be a contributing factor to prevent or diminished any superficial visceral pain problem which does not need a therapeutic treatment.

In an embodiment, the nutritional supplement composition further comprises at least one acceptable nutritional ingredient, preferably chosen among: probiotics, prebiotics, symbiotics, parabiotics, metabiotics, vitamins, minerals, herbs, amino acids and enzymes.

Probiotics intends according to the invention to live microorganisms that, when administered in adequate amounts, confer a health benefit on the host.

More preferably, said probiotics are chosen among bacteria belonging to Bifidobacterium, Lactobacillus, Lactococcus, Streptococcus genus or next generation probiotics such as bacterial strains identified as Faecalibacterium prausnitzii, Akkermansia muciniphila, Bacteroides fragilis, Bacteroides thetaiotaomicron, Clostridium butyricum, Roseburia hominis, Roseburia intestinalis, Hafnia alvei, or Christensenella minuta. It comprises also Saccharomyces fungi strains.

Prebiotics intends according to the invention to selectively fermented ingredients that results in specific changes in the composition and/or activity of the gastrointestinal microbiota, thus conferring benefit(s) upon host health.

More preferably, said prebiotics are chosen among Galactooligosaccharides, fructooligosaccharides or polysaccharides.

Symbiotics intends according to the invention to a mixture of probiotics and prebiotics that beneficially affects the host by improving the survival and activity of beneficial microorganisms in the gut.

More preferably, said synbiotics are chosen among bacteria belonging to Bifidobacterium, Lactobacillus, Lactococcus, Streptococcus genus or next generation probiotics such as bacterial strains identified as Faecalibacterium prausnitzii, Akkermansia muciniphila, Bacteroides fragilis, Bacteroides thetaiotaomicron, Clostridium butyricum, Roseburia hominis, Roseburia intestinalis, Hafnia alvei, or Christensenella minuta. It comprises also Saccharomyces fungi strains. All these bacteria or fungi should be associated with prebiotics such as galacooligosaccharides, fructooligosaccharides or polysaccharides in symbiotic formulation.

Parabiotics, paraprobiotics or inactivated probiotics intends according to the invention to non-viable microbial cells (either intact or broken) or crude cell extracts which when administered (either orally or topically) in adequate amounts, confer a benefit on the human or animal consumer.

More preferably, said parabiotics are chosen among: Inactivated microbial cells belonging to Lactobacillus, Lactococcus, Streptococcus or Bifidobacterium genus or based on Faecalibacterium prausnitzii, Akkermansia muciniphila, Bacteroides fragilis, Bacteroides thetaiotaomicron, Clostridium butyricum, Roseburia hominis, Roseburia intestinalis, Hafnia alvei, or Christensenella minuta bacterial species or Saccharomyces fungi strains.

Metabiotics or “postbiotics” are bioactive molecules produced by probiotics which provides physiological benefits to the host, by regulating the interactions between the host and the gut microbiome, for example through epigenetic regulation and cell communication. They can be found in any product which has been fermented by living bacteria.

Preferably, said metabiotics are chosen among products which have been fermented with living bacteria.

In an embodiment, the nutritional supplement composition according to the invention comprises at least 10⁹, preferably at least 10¹⁰, colony forming units/mL of the Parabacteroides distasonis strain deposited under accession number CNCM I-5828.

In an embodiment, the administration of this nutritional supplement is by oral take.

In an embodiment, the taken of the nutritional supplement is made by one or several successive doses.

In an embodiment, the successive doses are separated by at least 12 hours, preferably at least one day, more preferably at least two days, even more preferably at least 3 days, even more preferably at least 4 days, even more preferably at least 5 days, even more preferably at least 6 days.

In an even more preferred embodiment, said successive doses are separated by at least 1 week.

In an embodiment, the oral take of the nutritional supplement is made by gelatin capsules, capsules, tablets, powders, granules, oral solutions or suspensions.

In an embodiment, the oral take of the nutritional supplement is made through food products, beverage, a food additive, or a milk product, containing said nutritional supplement.

In an embodiment, the nutritional supplement composition according to the invention only comprises living bacteria.

In an embodiment, the nutritional supplement composition according to the invention only comprises killed bacteria and/or fractions of bacteria obtained by thermal, chemical or mechanical inactivation or UV inactivation that could be fractionated or purified.

According to the invention, a “killed bacteria” is a bacteria strain which is inactivated and which is no longer able to replicate.

The killed bacteria could be obtain according to the invention by heating treatment at 121° C. during 15 to 30 minutes.

In an embodiment, the nutritional supplement composition according to the invention comprises living bacteria and/or killed bacteria and/or fractions of bacteria.

In a fourth aspect, the invention concerns a composition comprising the bacterial strain according to the invention, for use as a medicament.

In an embodiment, the composition is for use in treating and/or preventing visceral pain.

In a preferred embodiment, said visceral pain is induced by Inflammatory Bowel Disease including Crohn's disease and Ulcerative Colitis, or by Irritable Bowel Syndrome.

“Visceral pain” according to the invention includes chronic abdominal pain, thoracic pain, pelvic pain, bloating associated with alteration of bowel habits, abdominal discomfort relieved with defecation, chronic pain within the digestive tract. According to the invention, this term includes pain related to the stomach, pancreas, liver, gallbladder, colon, and small and large intestine.

According to the invention, the term “preventing” indicates a reduction of the risk to develop the said pathologies.

According to the invention, the term “treating” means the alleviation of the symptoms associated with said specific disorders or conditions and/or elimination of said symptoms.

As shown in the examples of this application, this bacterial strain exhibits unusual anti-nociceptive properties by reducing visceral hypersensitivity in inflammatory and non-inflammatory mouse model that was not observed with the reference bacterial strain belonging to the same species. In addition, P. distasonis F1-2 strain exerts unusual neuromodulatory properties that contributes to decrease pain processing and reduces the severity of visceral pain, including antalgic effect.

Moreover, this composition reduces pain observed in IBS. Finally, this strain of Parabacteroides distasonis has neuroinhibitory properties on nociceptors expressing TRPV1 channel and G-coupled protein receptors.

In an embodiment the composition for use according to the invention comprises at least 10⁹, preferably at least to 10¹⁰ colony forming units/mL of the Parabacteroides distasonis strain deposited under accession number CNCM I-5828.

In an embodiment the composition for use according to the invention only comprises living bacteria.

In an embodiment the composition for use according to the invention only comprises killed bacteria and/or fractions of bacteria.

In an embodiment the composition for use according to the invention comprises living bacteria and/or killed bacteria and/or fractions of bacteria.

In an embodiment, the composition for use according to the invention further comprises at least one other acceptable active pharmaceutical ingredient, and/or at least one acceptable pharmaceutical excipient and/or at least one acceptable pharmaceutical carrier.

As acceptable active pharmaceutical ingredient is intended according to the invention every compound, molecule or else which can be useful to add into the composition to reach the purpose of the composition according to the invention. This definition includes especially anti-inflammatory molecules such as for example naproxen, aspirin or acetaminophen, as well as opioids such as codeine or morphine, or painkiller.

As acceptable pharmaceutical excipient is intended according to the invention any substances other than the active ones, that have been appropriately evaluated for safety and are intentionally included in a formulation. These excipients enable the medicament to be effectively administered to the patient, and to support the right delivery into the body.

As acceptable pharmaceutical carrier is intended according to the invention any substrate which serves to improve the selectivity, effectiveness, and/or safety of the administration of the composition according to the invention.

In an embodiment, the composition for use according to the invention is formulated to be suitable for oral, intravenous, in intestine or subcutaneous administration.

In a preferred embodiment, the composition for use according to the invention is administered orally, through gelatin capsules, capsules, tablets, powders, granules, oral solutions or suspensions.

In an embodiment, the administration is made by one or several successive doses.

In an embodiment, the successive doses are separated by at least 12 hours, preferably at least one day, more preferably at least two days, even more preferably at least 3 days, even more preferably at least 4 days, even more preferably at least 5 days, even more preferably at least 6 days.

Preferably, said successive doses are separated by at least 1 week.

In an embodiment, the subject is a mammal, including non-human mammal, and is, in particular, a human being.

In a preferred embodiment, the subject is a human adult.

In another aspect, the invention concerns a cell of the Parabacteroides distasonis strain deposited under accession number CNCM I-5828.

In another aspect, the invention concerns a cell of the Parabacteroides distasonis strain deposited under accession number CNCM I-5828, for use as a medicament.

In an embodiment, the cell according to the invention is for use in treating and/or preventing visceral pain.

More preferably, said visceral pain is induced by Inflammatory Bowel Disease including Crohn's disease and Ulcerative Colitis, or by Irritable Bowel Syndrome.

In another aspect, the invention concerns a nutritional supplement composition comprising a cell of the Parabacteroides distasonis strain deposited under accession number CNCM I-5828.

In another aspect, the invention concerns a method of preventing and/or treating visceral pain, comprising the administration of Parabacteroides distasonis strain deposited under accession number CNCM I-5828.

In a preferred embodiment, the method of preventing and/or treating according to the invention is to prevent and/or treat visceral pain induced by Inflammatory Bowel Disease including Crohn's disease and Ulcerative Colitis, or by Irritable Bowel Syndrome.

In an embodiment, the method according to the invention comprises the administration of at least 10⁹, preferably at least 10¹⁰, colony forming units/mL of the Parabacteroides distasonis strain deposited under accession number CNCM I-5828.

In another aspect, the invention concerns a strain of Parabacteroides distasonis which has a 16s rRNA gene sequence that is at least 80.00% identical to the 16s RNA sequence of a bacteria strain of the species Parabacteroides distasonis chosen among Parabacteroides distasonis ATCC 8503 or SEQ ID NO:1.

In a preferred embodiment, said strain has a 16s rRNA gene sequence that is at least 85.00%, more preferably at least 90.00%, even more preferably at least 95.00%, identical to the 16s RNA sequence of a bacteria strain of the species Parabacteroides distasonis chosen among Parabacteroides distasonis ATCC 8503 or SEQ ID NO:1.

In a preferred embodiment, said strain has a 16s rRNA gene sequence that is at least 96.00%, more preferably at least 97.00%, even more preferably at least 98.00%, identical to the 16s RNA sequence of a bacteria strain of the species Parabacteroides distasonis chosen among Parabacteroides distasonis ATCC 8503 or SEQ ID NO:1.

In a preferred embodiment, said strain has a 16s rRNA gene sequence that is at least 99.00%, more preferably at least 99.50%, identical to the 16s RNA sequence of a bacteria strain of the species Parabacteroides distasonis chosen among Parabacteroides distasonis ATCC 8503 or SEQ ID NO:1.

In a preferred embodiment, said strain has a 16s rRNA gene sequence that is at least 99.61%, identical to the 16s RNA sequence of the bacteria strain Parabacteroides distasonis ATCC 8503 or SEQ ID NO:1.

In a preferred embodiment, said strain has a 16s rRNA gene sequence as described in SEQ. ID NO:1.

In another aspect, the invention concerns a composition comprising a strain of Parabacteroides distasonis which has a 16s rRNA gene sequence that is at least 80.00% identical to the 16s RNA sequence of a bacteria strain of the species Parabacteroides distasonis chosen among Parabacteroides distasonis ATCC 8503 or SEQ ID NO:1.

In an embodiment, the composition according to the invention comprises at least 10⁹, preferably at least 10¹⁰, colony forming units/mL of a strain of Parabacteroides distasonis which has a 16s rRNA gene sequence that is at least 80.00% identical to the 16s RNA sequence of a bacteria strain of the species Parabacteroides distasonis chosen among Parabacteroides distasonis ATCC 8503 or SEQ ID NO:1.

In an embodiment, said composition comprises a strain which is at least 85.00%, preferably at least 90.00%, more preferably at least 95.00%, more preferably at least 96.00%, more preferably at least 97.00%, more preferably at least 98.00%, more preferably at least 99.00%, more preferably at least 99.50%, more preferably at least 99.61%, identical to the 16s RNA sequence of a bacteria strain of the species Parabacteroides distasonis chosen among Parabacteroides distasonis ATCC 8503 or SEQ ID NO:1.

In another aspect, the invention concerns a nutritional supplement composition comprising a strain of Parabacteroides distasonis which has a 16s rRNA gene sequence that is at least 80.00% identical to the 16s RNA sequence of a bacteria strain of the species Parabacteroides distasonis chosen among Parabacteroides distasonis ATCC 8503 or SEQ ID NO:1.

In an embodiment, the nutritional supplement composition according to the invention comprises at least 10⁹, preferably at least 10¹⁰, colony forming units/mL of a strain of Parabacteroides distasonis which has a 16s rRNA gene sequence that is at least 80.00% identical to the 16s RNA sequence of a bacteria strain of the species Parabacteroides distasonis chosen among Parabacteroides distasonis ATCC 8503 or SEQ ID NO:1.

In an embodiment, said nutritional supplement composition comprises a strain which is at least 85.00%, preferably at least 90.00%, more preferably at least 95.00%, more preferably at least 96.00%, more preferably at least 97.00%, more preferably at least 98.00%, more preferably at least 99.00%, more preferably at least 99.50%, more preferably at least 99.61%, identical to the 16s RNA sequence of a bacteria strain of the species Parabacteroides distasonis chosen among Parabacteroides distasonis ATCC 8503 or SEQ ID NO:1.

In another aspect, the invention concerns a composition comprising a strain of Parabacteroides distasonis which has a 16s rRNA gene sequence that is at least 80.00% identical to the 16s RNA sequence of a bacteria strain of the species Parabacteroides distasonis chosen among Parabacteroides distasonis ATCC 8503 or SEQ ID NO:1., for use as a medicament.

In an embodiment, the composition for use according to the invention comprises at least 10⁹, preferably at least 10¹⁰, colony forming units/mL of a strain of Parabacteroides distasonis which has a 16s rRNA gene sequence that is at least 80.00% identical to the 16s RNA sequence of a bacteria strain of the species Parabacteroides distasonis chosen among Parabacteroides distasonis ATCC 8503 or SEQ ID NO:1.

In an embodiment, said composition for use comprises a strain which is at least 85.00%, preferably at least 90.00%, more preferably at least 95.00%, more preferably at least 96.00%, more preferably at least 97.00%, more preferably at least 98.00%, more preferably at least 99.00%, more preferably at least 99.50%, more preferably at least 99.61%, identical to the 16s RNA sequence of a bacteria strain of the species Parabacteroides distasonis chosen among Parabacteroides distasonis ATCC 8503 or SEQ ID NO:1.

In an embodiment, said composition is for use in treating and/or preventing visceral pain. In an embodiment, said visceral pain is induced by Inflammatory Bowel Disease including Crohn's disease and Ulcerative Colitis, or by Irritable Bowel Syndrome.

References to a percentage sequence identity between two nucleotide sequences means that, when aligned, that percentage of nucleotides are the same in comparing the two sequences.

In another aspect, the invention concerns a cosmetic skincare composition comprising a Parabacteroides distasonis strain deposited under accession number CNCM I-5828.

In an embodiment, said composition comprises at least 10⁹, preferably at least 10¹⁰, colony forming units/mL of the Parabacteroides distasonis strain deposited under accession number CNCM I-5828.

In an embodiment, said composition is administered by topical application.

By “topical application” is meant in the sense of the present invention an application to the skin (including the scalp), and the mucous membranes.

In an embodiment, said cosmetic composition also comprises at least one other cosmetically acceptable agent, preferably chosen from soothing agents, restructuring agents, regenerating agents, revitalizing agents, sunscreens, anti-wrinkle agents, moisturizing agents, anti-aging agents, surfactants, fats, organic solvents, solubilizing agents, thickening and gelling agents, smoothing agents, firmness-enhancing agents, elasticity and/or the barrier effect of the skin, antioxidants, opacifiers, thermal waters, mattifying agents, chemical or mineral filters, trace elements, stabilizing agents, foaming agents, perfumes, ionic or non-ionic emulsifiers, fillers, sequestrants and chelators, perfumes, filters, essential oils, coloring matters, pigments, hydrophilic or lipophilic active ingredients, lipid vesicles encapsulating one or more active ingredients and/or preservatives.

By “cosmetically acceptable” is meant according to the present invention a compound which is useful in the preparation of a cosmetic composition, is generally safe, non-toxic and neither biologically nor otherwise undesirable, and is acceptable for cosmetic use, particularly by topical application to the skin.

Preferably, said cosmetic composition is formulated in the form of a cream, an ointment, a balm, a mask, a milk, a lotion, a serum, a spray, a paste, a foam, an aerosol, a stick, a shampoo, a conditioner, patches, an aqueous-alcoholic or oily solution, an oil-in-water or water-in-oil or multiple emulsion, an aqueous or oily gel, a liquid, pasty or solid anhydrous product, and/or a dispersion of oil in an aqueous phase with the aid of spherules, it being possible for these spherules to be polymeric nanoparticles, such as nanospheres and nanocapsules, or lipid vesicles of ionic and/or nonionic type.

In an embodiment, an amount of 0.2 to 3 mg/cm2 of the cosmetic composition is applied to the skin area, preferably 2 mg/cm2.

In an embodiment, the cosmetic composition according to the invention is applied once to twice a day, for at least 7 days, preferably at least 15 days, even more preferably at least one month and most preferably at least 2 months. Most preferably, the cosmetic composition is applied in the method according to the invention twice a day for at least 2 months.

In another aspect, the invention concerns a dermocosmetic composition for use in the treatment of atopic dermatitis and/or psoriasis.

In an embodiment, said composition comprises at least 10⁹, preferably at least to 10¹⁰, colony forming units/mL of the Parabacteroides distasonis strain deposited under accession number CNCM I-5828.

According to one embodiment, said composition further comprises at least one other dermatologically active principle.

More particularly, the composition according to the present invention also comprises at least one other dermatologically active principle acting either on the reinforcement of the dermal-epidermal junction, the cell-matrix adhesion of the skin, and/or the cell-cell adhesion and/or the migration of the keratinocytes, or otherwise on the skin, depending on the nature of the agent(s) used, such as a moisturizing agent, a lipid-replenishing agent a superfatting agent, an exfoliating agent, a keratolytic agent, an antioxidant agent, a soothing agent, a softening agent, a sedative agent, a cleansing agent, a makeup remover, a sanitizing agent, an antibacterial agent, an antiseptic agent, an antiseborrheic agent, a decongesting agent, a revitalizing agent, a cell renewal activating agent or one or more sunscreens.

The compositions of the invention must be in a dermatologically acceptable form, i.e. compatible with the skin, hair and/or hair. In an embodiment, said dermocosmetic composition is administered by topical application.

Thus, preferably, the said dermocosmetic composition according to the invention is in the form of a cream, an ointment, an ointment, a mask, a serum, a milk, a lotion, a paste, a foam, an aerosol, a stick, a powder, a solution, a suspension, a shampoo, a conditioner, patches, an aqueous-alcoholic or oily solution, an oil-in-water or water-in-oil or multiple emulsion, an aqueous or oily gel, a liquid, pasty or solid anhydrous product, and/or a dispersion of oil in an aqueous phase with the aid of spherules, it being possible for these spherules to be polymeric nanoparticles, such as nanospheres and nanocapsules, or lipid vesicles of the ionic and/or nonionic type.

In an embodiment, an amount of 0.2 to 3 mg/cm2 of the dermocosmetic composition is applied to the skin area.

In an embodiment, the cosmetic composition according to the invention is applied once to twice a day, for at least 7 days, preferably at least 15 days, even more preferably at least one month and most preferably at least 2 months. Most preferably, the cosmetic composition is applied in the method according to the invention twice a day for at least 2 months.

In another aspect, the invention concerns the use of a Parabacteroides distasonis strain deposited under accession number CNCM I-5828 in agronomy as a biofertilizers and/or biocontrol agent.

A biofertilizer agent is a product which contains living microorganisms that, when applied to soil, seeds, or surfaces of plant, colonize the rhizosphere or the plant internal tissues and induce plant growth.

A biocontrol agent is a product which replace pesticides to manage plant diseases by using living entities such as microorganisms.

In an embodiment, said use as a biofertilizers and/or biocontrol is done through a composition comprising at least 10⁹, preferably at least 10¹⁰, colony forming units/mL of a strain of a Parabacteroides distasonis strain deposited under accession number CNCM I-5828.

### BRIEF DESCRIPTION OF DRAWINGS

FIG. 1: P. distasonis F1-2 reverses colonic hypersensitivity in dextran sulfate sodium 0.5% colitis induced mouse model. (a) Visceromotor response to colorectal distension in control (vehicle) mice (n=8), DSS treated mice (n=10) and P. distasonis F1-2 treated mice (n=9). (b) The area under curve (AUC) was calculated between 60 and 80 mmHg for the different groups. P. distasonis F1-2 strain administration significantly alleviated colonic hypersensitivity induced by DSS treatment in mice. Mean+/−SEM. Statistical analysis: (a) Two-way ANOVA followed by Dunnett's multiple comparisons test. DSS 0.5%+veh vs Water+veh treated mice: ***p value=0.0002 (at 80 mmHg); ***p value=0.0006 (at 60 mmHg); ***p value=0.0002 (at 40 mmHg). DSS 0.5%+F1-2 vs Water+veh treated mice: no significant (40 mmHg p value=0.1945; 60 mmHg p value=0.2234; 80 mmHg p value=0.0589. (b) One-way ANOVA followed by Tukey's multiple comparisons test. Water+veh vs DSS 0.5%+veh treated mice: ***p value=0.0002; Water+veh vs DSS 0.5%+F1-2: no significant (p value=0.2015); DSS 0.5%+veh vs DSS 0.5%+F1-2 treated mice: *p value=0.0108.

FIG. 2: Administration of Parabacteroides distasonis F1-2 but not P. distasonis ATCC 8503 prevents colonic hypersensitivity in DSS-induced colitis. (a) Visceromotor response to colorectal distension in control (water+vehicle) mice (n=9), DSS 0.5%+vehicle treated mice (n=11), DSS 0.5%+P. distasonis F1-2 (n=10) or DSS 0.5%+P. distasonis ATCC 8503 (n=10) treated mice was assessed at D12. (b) The area under curve (AUC) was calculated between 60 and 80 mmHg for the different groups. Mean+/−SEM. Statistical analysis: (a) Two-way ANOVA followed by Tukey's multiple comparisons test. Water+veh vs DSS 0.5%+veh treated mice: ****p value<0.0001 (at 80 mmHg); ***p value=0.001 (at 60 mmHg); *p value=0.0243 (at 40 mmHg). Water+veh vs DSS 0.5%+ATCC 8503 treated mice: ****p value<0.0001 (at 80 mmHg); **p value=0.0012 (at 60 mmHg); *p value=0.0343 (at 40 mmHg). Water+veh vs DSS 0.5%+F1-2 treated mice: ns (at 80, 60 and 40 mmHg).

FIG. 3: Parabacteroides distasonis F1-2 alleviates colonic hypersensitivity in post-inflammatory DNBS colitis induced mouse model. (a) Visceromotor response to colorectal distension in EtOH+vehicle mice (n=7), DNBS+vehicle treated mice (n=8) and DNBS+P. distasonis F1-2 treated mice (n=10). (b) Area under curve (AUC) was calculated between 60 and 80 mmHg for the different groups. P. distasonis F1-2 administration significantly alleviated colonic hypersensitivity induced by DNBS treatment. Mean+/−SEM. Statistical analysis: (a) Two-way ANOVA followed by Dunnett's multiple comparisons test. EtOH 30%+veh vs DNBS+veh treated mice: ***p value=0.0003 (at 80 mmHg); **p value=0.0038 (at 60 mmHg). DNBS+F1-2 vs EtOH 30%+veh treated mice: no significant 80 mmHg p value=0.2504; 60 mmHg p value=0.7811. (b) One-way ANOVA followed by Tukey's multiple comparisons test. EtOH 30%+veh vs DNBS+veh treated mice: ****p value<0.0001. EtOH 30%+veh vs DNBS+F1-2 treated mice: ns (p value=6270). DNBS+veh vs DNBS+F1-2 treated mice: ****p value<0.0001.

FIG. 4: P. distasonis F1-2 alleviates colonic hypersensitivity in post-infectious IBS mouse model. (a) Visceromotor response to colorectal distension in uninfected mice (n=8), Infected+vehicle mice (n=9) and P. distasonis F1-2 infected treated mice (n=7). (b) Area under curve between 60 and 80 mmHg for the different groups. Colonic hypersensitivity developed by infected mice in post-infectious phase was reversed by P. distasonis F1-2 treatment. Mean+/−SEM. Statistical analysis: (a) Two-way ANOVA followed by Tukey's multiple comparisons test. Uninfected+veh vs Infected+veh treated mice: **p value=0.0028 (at 80 mmHg); *p value=0.0202 (at 60 mmHg). Infected+veh vs Infected+F1-2 treated mice: ##p value=0.002. (b) Kruskal-Wallis test. Uninfected+veh vs Infected+veh treated mice: *p value=0.0267. Infected+veh vs Infected+F1-2 treated mice: *p value=0.0355. Uninfected+veh vs Infected+F1-2: no significant (p value>0.99).

FIG. 5: P. distasonis F1-2 decreases nociceptors activation level. (a) Experimental protocol. Schematic representation of activation signal observed by ratiometric calcium imaging with 2 sub populations. First one corresponds to neuronal cells responding both to the first and the second stimulation (in black). The second corresponds to neuronal cells that responding only to the first stimulation (in grey). Neuronal cells were stimulated a first time with capsaicin (1 μM) or IS before being incubated with Tyrode or P. distasonis F1-2. Neuronal cells were stimulated a second time with capsaicin or IS mixed with P. distasonis F1-2 before being lastly stimulated with KCl (50 mM). IS was composed by bradykinin, serotonin, histamine, PGE2 and KCl. (b) % of sub population of neuronal cells activated by capsaicin. (c) Global kinetic signal activation measured as normalized fluorescence 340 nm/380 nm for capsaicin positive responding cells in each group. (d) % of sub population of neuronal cells activated by IS. (e) Global kinetic signal activation measured as normalized fluorescence 340 nm/380 nm for IS positive responding cells in each group. Statistical analysis: Chi square test ****p value<0.0001. GPCR: G-protein coupled receptor. IS: Inflammatory soap.

FIG. 6: P. distasonis F1-2 neuroinhibitory potential could involve G-protein coupled receptor. (a) Dose-response curve of bradykinin on ND7/23 expressing bradykinin receptor incubated with or without P. distasonis F1-2 or P. distasonis ATCC 8503 strain. (b) Experimental protocol. Schematic representation of Ca²⁺ activation signal of ND7/23 neuronal cells loaded with calcium probe (FLIPR Calcium 6 QF) stimulated with bradykinin (40 nM) after 45 min incubation with P. distasonis F1-2. (c) Activation signal of ND7/23 neuronal cells stimulated with bradykinin after 45 min incubation with P. distasonis F1-2 with extracellular Ca²⁺ or (d) in deprivation of extracellular Ca²⁺. (e) Relative response to control. Bar graphs represent maximum peaks values on normalized F-FQ relative to control. (f) Relative response to bradykinin stimulation of ND7/23 overnight pre-treated or not with pertussis toxin. Statistical analysis: (a) Mean+/−SEM. Two-way ANOVA followed by Dunnett's multiple comparisons test. P. distasonis F1-2 versus vehicle: *p value=0.0191 (at 10⁻¹¹M); **p value=0.0016 (at 10⁻¹⁰M); ***p value=0.0002 (at 10⁻⁹M); *p value=0.0205 (at 10⁻⁸M); *p value=0.0222 (at 10⁻⁷M); **p value=0.0091 (at 10⁻⁵M); *p value=0.0165 (at 10⁻⁴M). P. distasonis ATCC 8503 versus vehicle: *p value=0.0157 (at 10⁻¹¹M); *p value=0.0266 (at 10⁻¹⁰M); **p value=0.0042 (at 10⁻⁹M). Each point represents an average of 4 independent readings. (e) One-way ANOVA followed by Tukey's multiple comparisons test. Vehicle (+) Ca²⁺ vs F1-2 (+) Ca²⁺: ***p value=0.0003. Vehicle (−) Ca²⁺ vs F1-2 (−) Ca²⁺: ****p value<0.0001. Each bar graph represents an average of 3 independent readings. (f) Mean+/−SEM. Two-way ANOVA followed by Sidak's multiple comparisons test. ****p value<0.0001. Each bar graph represents an average of 6 independent readings.

FIG. 7: P. distasonis F1-2 neuroinhibitory potential could involve bradykinin receptor. (a) Experimental protocol. Schematic representation of activation signal observed by ratiometric calcium imaging with 2 sub populations. First one corresponds to neuronal cells responding both to the first and the second stimulation (in black). The second corresponds to neuronal cells that responding only to the first stimulation (in grey). Ca²⁺ responses were measured on neuronal cells from DRG mice as normalized fluorescence 340 nm/380 nm ratios. (b) % of sub population of neuronal cells activated by IS (+) bradykinin, IS (−) bradykinin or bradykinin. (c) % of sub population of neuronal cells activated by bradykinin or by histamin receptor 1 specific agonist (H1R) (2-pyridylethylamine). F1-2: P. distasonis F1-2; Stim: stimulation; IS: Inflammatory soap; Brady: bradykinin; H1R: histamine receptor 1 specific agonist. Statistical analysis: Chi square test. ****p value<0.0001.

### EXAMPLES

In the following examples, colonic hypersensitivity in animal models is measured to evaluate the efficacy of the strain according to the invention on chronic visceral pain in human.

### Example 1: Parabacteroides distasonis F1-2 Strain has Antalgic Effect in Dextran Sulfate Sodium Acute Colitis Mouse Model (Inflammatory Visceral Pain Mouse Model)

Parabacteroides distasonis Culturing Methods

The strain Parabacteroides distasonis (isolate F1-2) has been cultivated in complex anaerobic medium containing Neutralized soya peptone (10 g/L, OXOID), Yeast extract (15 g/L, OXOID), KH₂PO₄ (0.9 g/L), K₂HPO₄ (0.9 g/L), NaCl (0.9 g/L), sodium acetate (2.7 g/L), MgSO₄, 7H2O (0.09 g/L), L-cysteine hydrochloride (1 g/L), D-glucose −20 g/L), NaHCO₃ (4 g/L), Maltose (0.5 g/L), Cellobiose (0.5 g/L), adjusted at pH 6.9. For inoculum preparation, 1 mL of P. distasonis F1-2 frozen stock culture was sub-cultured in a 50 mL flask containing 25 mL of anaerobic medium (previously described) and incubated 24 h at 37° C. in strict anaerobic conditions. A second sub-cultured was necessary and consists to transferred 24 h first sub-cultured (OD600 nm of 3-4) in 225 mL of anaerobic medium, incubated for 20 h at 37° C. in strict anaerobic conditions. Cultivation with growing cells was carried out in 5 L bioreactor by inoculated 5% (v/v) of the second sub-cultured (OD600 nm=3) in the fresh anaerobic medium. Anaerobic conditions were maintained by continuous injection of a gas mixture (N₂ (85%), H₂ (5%) and CO₂ (10%)) allowing to maintain low Redox potential (−400 mV) and homogenization was ensured by 2 Rushton blades with an agitation rate of 150 rpm. Bioreactor culture was conducted during 24 h with a controlled temperature of 37° C. and reached OD600 nm of 6. Biomass of P. distasonis F1-2 was harvested by centrifuging whole culture 30 minutes at 5000 g at 4° C. in anaerobic condition to maintain cell viability and conserved in PBS+glycerol (20% (v/v)) solution at −80° C. Dry matter of biomass was adjusted to 19% with PBS+glycerol (20%) to standardized bacterial preparation. Bacterial titer was estimated between 1.2×10¹⁰ CFU/mL to 1.5×10¹⁰ CFU/mL according to the batch production by count on brucella blood agar medium (from anaerobe system).

### Animals

57 Male C57/Blk6 mice (6-9 weeks old) were obtained from Janvier Labs (53 Le Genest-Saint-Isle. France).

### Animal Treatments

Acute colitis was induced by 0.5% (w/v) Dextran Sulfate Sodium (DSS) (mol wt=36,000-50,000 Da) dissolved in drinking water for 12 days ad libitum. During 12 days, 0.2 mL of 1.52×10¹⁰ CFU/mL of P. distasonis F1-2 strain were daily administered by gavage. Animal weight loss was controlled during DSS administration and feces were harvested each day of the experiment and frozen at −80° C. for further analysis. The study groups are as follows: control (water+vehicle group (PBS+glycerol (20%)), DSS+vehicle (PBS+glycerol (20%)) and DSS+F1-2 group. Colonic hypersensitivity was measured at the end of the experiment. In a second experiment, P. distasonis F1-2 strain antalgic effect was compared to P. distasonis ATCC 8503 reference strain following the same procedure.

### Measurement Colonic Hypersensitivity by Colorectal Distension

Mice colonic sensitivity induced by DSS treatment was evaluated by quantifying visceromotor reaction in response to colorectal distensions (CRD). Mice were habituated 30 min prior to experiments, to reduce motion artefacts due to restraint stress. A polyethylene balloon with connecting catheter was inserted in the distal colon, 1 cm from the base of the balloon to the anus of mice, during light isoflurane anesthesia. The catheter was fixed to the tail with tape. The balloon was connected to pressure transducer to control intraballoon pressure during CRD procedure. Barostat was used to manage air infiltration and balloon pressure control. The CRD procedure allows assessing visceral pain-related responses and consisted of repeated phasic distensions of 20, 40, 60 and 80 mmHg, with a pulse duration of 20 seconds and spaced from each other by 4 minutes.

### Results—Conclusions

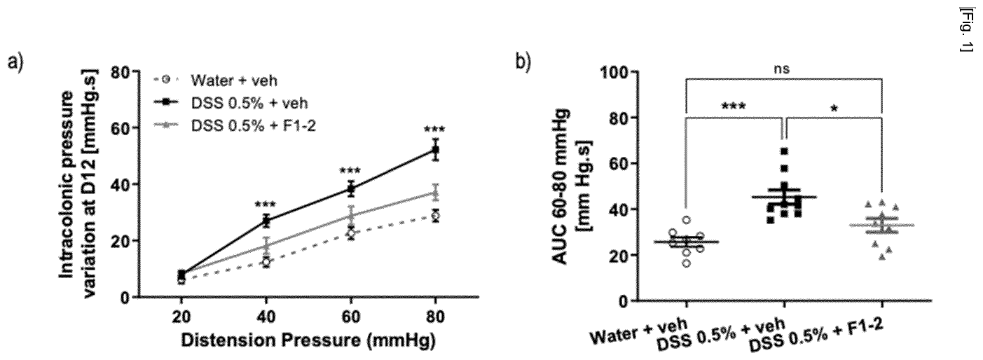

To evaluate anti-nociceptive effect of the strain P. distasonis F1-2 on colonic hypersensitivity, a model of colitis induced by 0.5% of Dextran sulfate sodium was used. P. distasonis F1-2 was administered by gavage each day throughout the duration of DSS treatment, so 12 consecutive days. While DSS 0.5% treatment increased significantly colonic hypersensitivity (***p value=0.0002 (at 80 mmHg)) (FIG. 1a) (***p value=0.0002) (FIG. 1b), P. distasonis F1-2 oral administration significantly decreased colonic hypersensitivity compared to control. In fact, there is no significant difference between Water+veh and DSS 0.5%+F1-2 mice group (FIG. 1a and FIG. 1b) traducing a partial reversion of the treatment-induced painful phenotype.

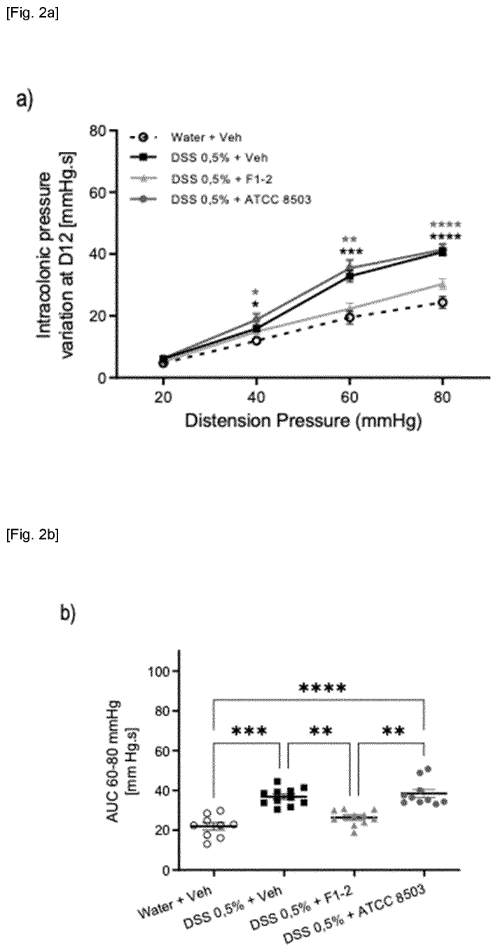

In a second experiment, anti-nociceptive effect of P. distasonis F1-2 strain was compared to P. distasonis ATCC 8503 reference strain in the same visceral pain mouse model, according to the same procedure. While P. distasonis F1-2 strain exerted significant anti-nociceptive effect in mouse treated with dextran sulfate sodium (Water+veh vs DSS 0.5%+F1-2: no significant) (FIG. 2a), P. distasonis ATCC 8503 reference strain had no beneficial effect on colonic hypersensitivity (Water+veh vs DSS 0.5%+ATCC 8503: ****p value<0.0001 (at 80 mmHg) (FIG. 2a et 2b).

### Example 2: Parabacteroides distasonis F1-2 Strain has Antalgic Effect in Dinitrobenzene Sulfonic Acid Colitis Mouse Model

Parabacteroides distasonis Culturing Methods

### Cf Example 1: Parabacteroides distasonis Culturing Methods

### Animals

25 Male C57/Blk6 mice (6-9 weeks old) were obtained from Janvier Labs (53 Le Genest-Saint-Isle. France).

### Animal Treatments

Colitis was induced by intrarectal instillation of Dinitrobenzene sulfonic acid (DNBS) (100 mg/Kg) dissolved in 30% (v/v) ethanol solution at day 1 and day 23. During 10 days (day 17 to 26), 0.2 mL of 1.52×10¹⁰ CFU/mL of P. distasonis F1-2 strain were daily administered by gavage. Animal weight loss was followed 4 days after each DNBS intrarectal instillation to control treatment efficacy. The study groups are as follows: control (EtOH 30%+vehicle (PBS+glycerol)) group, DNBS+vehicle group (PBS+glycerol) and DNBS+F1-2 group. Colonic hypersensitivity was measured at the end of the experiment.

### Measurement Colonic Hypersensitivity by Colorectal Distension

### Cf Example 1: Measurement Colonic Hypersensitivity by Colorectal Distension

### Results—Conclusions

To evaluate anti-nociceptive effect of Parabacteroides distasonis, F1-2 strain was tested in a post-inflammatory visceral pain mouse model induced by rectal instillation of DNBS. DNBS induced a transient increase of intestinal permeability and inflammatory response during 1 to 3 days after instillation while colonic hypersensitivity was affected for a longer duration.

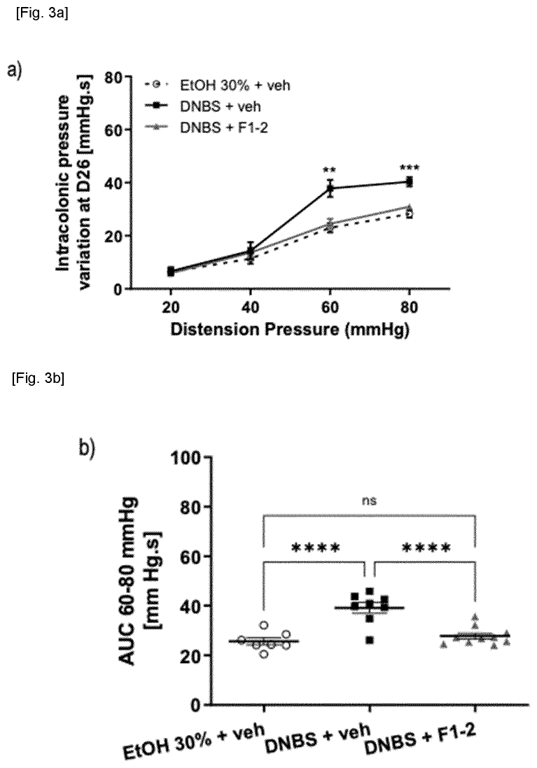

Beneficial effect of P. distasonis F1-2 was observed in post-inflammatory DNBS colitis induced mouse model (FIG. 3). In fact, while DNBS rectal instillation caused colonic hypersensitivity (FIG. 3a) (***p value=0.0003 (at 80 mmHg) and **p value=0.0038 (at 60 mmHg)), P. distasonis F1-2, administered during 10 days allowed to reverse pain phenotype (FIG. 3a) (EtOH 30%+veh vs DNBS+F1-2; ns). This is also the case when we calculated AUC (FIG. 3b).

### Example 3: P. distasonis F1-2 Strain has Antalgic Effect in IBS Post-Infectious Visceral Pain Mouse Model

### Parabacteroides Distasonis Culturing Methods

### Cf Example 1: Parabacteroides Distasonis Culturing Methods

### Animals

24 Male C57/Blk6 mice (6-9 weeks old) were obtained from Janvier Labs (53 Le Genest-Saint-Isle. France).

### Animal Treatments

After static overnight growth in a flask containing Luria broth, Citrobacter rodentium (ATCC 51459™ DBS100) cells were harvested by centrifugation and suspended in PBS buffer to reach standardized load cells of 5×10⁹ CFU/mL. At day 0, each mouse has been infected with 1×10⁹ CFU of C. rodentium. Infectious phase occurred before being totally clearing at day 16. Clearance of the pathogen was confirmed by counting C. rodentium in feces on MacConkey agar. P. distasonis F1-2 strain was daily-administered (3.03×10⁹ CFU/mice) by gavage at day 16 until day 23. The study groups are as follows: control (uninfected+vehicle (PBS+glycerol)) group (n=8), Infected mice+vehicle (PBS+glycerol) (n=9) and Infected mice+P. distasonis F1-2 strain (n=7). Colonic hypersensitivity was measured at the end of the experiment.

### Measurement Colonic Hypersensitivity by Colorectal Distension

### Cf Example 1: Measurement Colonic Hypersensitivity by Colorectal Distension

### Results—Conclusions

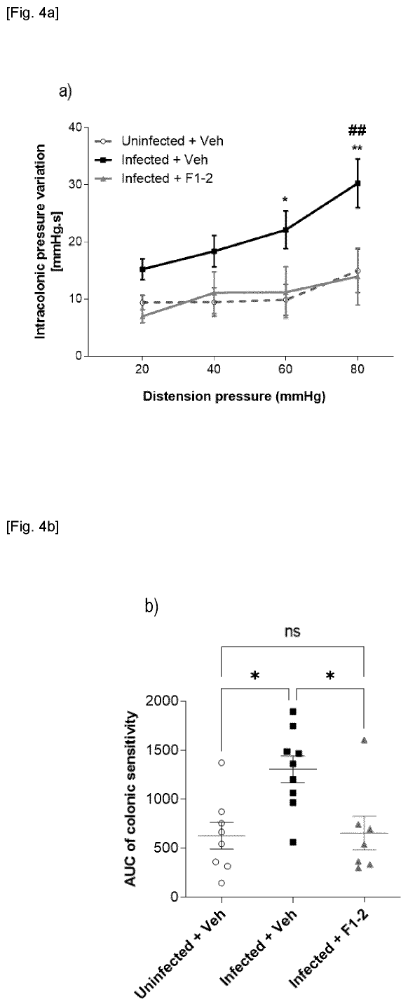

As colonic hypersensitivity can occur after gastro-intestinal infectious episode, antalgic potential of P. distasonis F1-2 strain was tested in post-infectious IBS mouse model induced by infection by the murine pathogen Citrobacter rodentium. 16 days post-infection, pathogen was totally cleared (data not shown) and mouse were daily gavaged with P. distasonis F1-2 bacterial strain during 8 days. In this model, mouse developed pain phenotype in post-infectious phase even though pathogen was totally cleared. In fact, significant colonic hypersensitivity was noted in infected group (FIG. 4a) (**p value=0.0028 (at 80 mmHg) and *p value=0.0202 (at 60 mmHg)) (FIG. 4b) (*p value=0.0267). Significant antalgic effect exerted by P. distasonis F1-2 was observed. In fact, colonic hypersensitivity developed by infected mouse was totally reversed by P. distasonis F1-2 treatment (FIG. 4a) (##p value=0.002 (at 80 mmHg)) (FIG. 4b) (Uninfected+veh vs Infected+F1-2: ns).

### Example 4: P. Distasonis F1-2 Decreases Nociceptors Activation Level

Parabacteroides distasonis Culturing Methods

### Cf Example 1: Parabacteroides distasonis Culturing Methods

### Culture of Primary Neuronal DRG Cells

Dorsal root ganglia from C57/Blk6 mouse (male) were explanted, cleaned of anterior and posterior roots and connective tissue and digested by enzyme mix containing Collagenase type III (5 mg/mL, Worthington) and Dispase (10 mg/mL; Gibco), for 45 minutes at 37° C. DRG suspension was centrifuged at 175 g for 15 seconds after which the supernatant was removed and replaced by 1 mL of Dulbecco's Modified Eagle Medium (DMEM; SIGMA). The DRG suspension was triturated through fire polished Pasteur pipettes and spined at 175 g 5 seconds. The supernatant containing dissociated cells was saved at each trituration step. Trituration was repeated 6 times using 3 fire polish Pasteur pipettes of decreasing diameter. Cell solution was spined for 5 min at 175 g. Supernatant was removed and replaced by 600 μL of DMEM supplemented with 10% (v/v) fetal bovine serum, sodium pyruvate (1 mM; Gibco), L-glutamine (2 mM), and penicillin (100 μg/mL), streptomycine (100 μg/mL), vitamins, amino acids (MEM NEAA, Gibco), and NGF (6.25 μg/mL). Cell suspension was plated on 12 poly-L-Lysine (100 μg/mL) and laminin (200 μg/mL) coated glass sheets in culture dishes. Cells were incubated during 30 minutes at 37° C. before adding 1 mL of DMEM supplemented medium in each well. Cells were incubated at 37° C. overnight.

### Evaluation of Neuronal Cells Activation by Ratiometric Calcium Imaging

Intracellular free Ca²⁺ was followed by ratiometric calcium imaging using Ca²⁺ ratiometric dye Fura-2 acetoxymethyl ester (Fura 2-AM, Invitrogen). DRG neurons were loaded with Fura-2 solution (4 μM) supplemented by pluronic acid (1 μg/mL) for 45 minutes at 37° C. with slight agitation (40 rpm). All imaging experiments were performed in a dark room temperature. After loading, glass coverslip was mounted in an imaging/perfusion chamber equipped with perfusion valve system which was mounted and viewed through an inverted microscope. Neuronal cells were alternatively illuminated with 340 nm and 380 nm wavelengths. The exposure time to excitation was 400 ms for each wavelength. Image pairs were acquired every 2 seconds. Calcium imaging experiment consisted on 2 stimulations spaced by 5 min incubation with 1.99 to 2.4×10⁸ P. distasonis F1-2 viable cells/mL after being rinsed with tyrode solution. Tyrode solution was composed as followed: NaCl (140 mM), KCl (3 mM), MgCl₂ (1 mM), CaCl₂) (2 mM), D-Glucose (10 mM), HEPES (10 mM) adjusted at pH7.4. Osmolarity was also adjusted at 300 mOsm. A lastly KCl (50 mM) stimulation was assessed at the end of experiment to evaluate cell viability. KCl solution was composed as followed: NaCl (93 mM), KCl (50 mM), MgCl₂ (1 mM), CaCl₂) (2 mM), D-Glucose (10 mM), HEPES (10 mM) adjusted at pH7.4. Osmolarity was also adjusted at 300 mOsm. In this experiment, capsaicin and P. distasonis F1-2 strain were diluted on tyrode physiological solution before used.

### Results—Conclusions

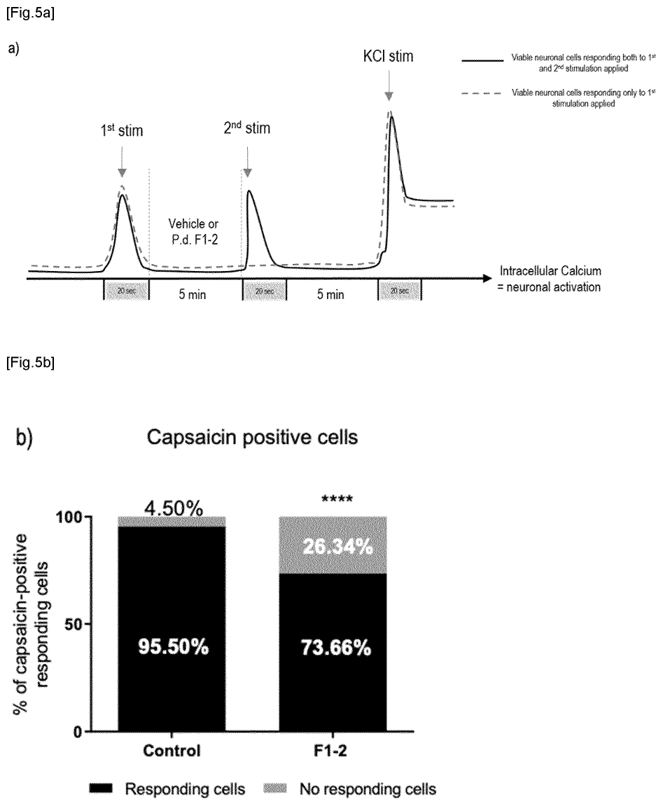

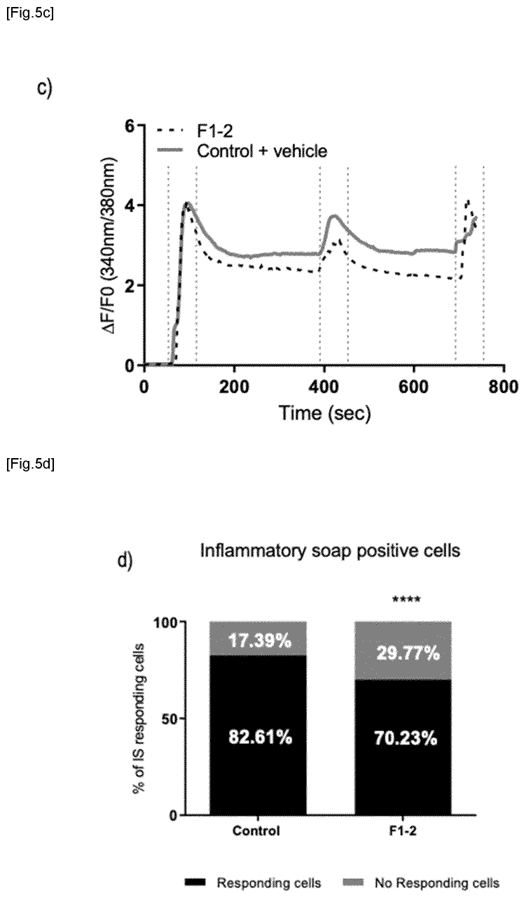

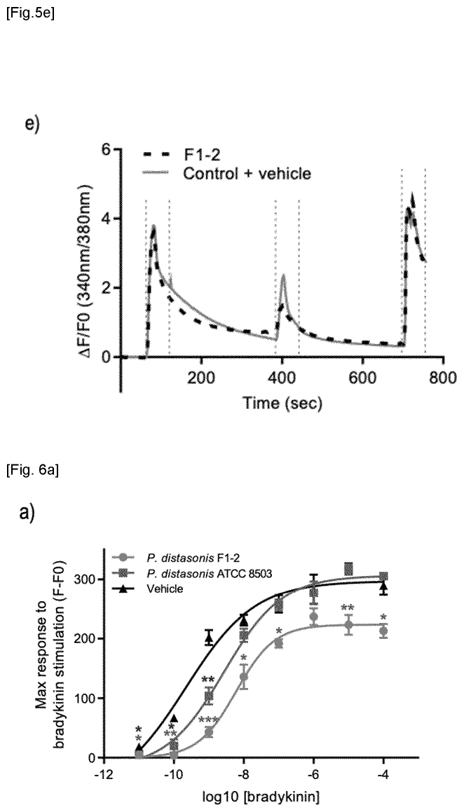

To understand molecular mechanisms taking place between P. distasonis F1-2 and nociceptors, neurons from DRG mice were cultivated and loaded with Fura-2 to follow neuronal activation under nociceptive stimulations. In these experiments, primary neuronal cells from DRG mice were stimulated a first time with stimulatory solution before being incubated with vehicle or P. distasonis F1-2 bacterial strain for 5 minutes. After that, neuronal cells were stimulated a second time with stimulatory solution mixed with vehicle or with P. distasonis F1-2 bacterial strain. A lastly KCl (50 mM) stimulation was made to control cell viability at the end of experiment (FIG. 5a).

In the first experiment, stimulatory agent consisted on capsaicin solution targeting specifically neuronal cells expressing TRPV1 channel, implied in visceral pain. Ca²⁺ responses (measured as normalized fluorescence 340 nm/380 nm ratios) of each neuronal cells was followed by ratiometric calcium imaging. Quantification of the average max ΔF/F0 of capsaicin-positive nociceptive neurons revealed 2 respective groups in each condition. In fact, in each condition tested, 2 sub populations within TRPV1 positive neurons were identified. First one corresponds to TRPV1 positive neurons that responded both to first and second stimulation; represented in black (FIG. 5b) whereas the second sub population corresponds to TRPV1 neurons that responded to the first stimulation only, represented in grey (FIG. 5b). Neuronal cells' incubation with P. distasonis F1-2 significantly increased the percentage of the second sub population by turning off 26.34% of TRPV1 cells versus 4.5% for control (FIG. 5b) (****p value<0.0001). This result was also reflected by a slightly calcium signal decreased in global kinetic (FIG. 5c).

In the second experiment, stimulatory agent consisted on inflammatory mixture solution comprising inflammatory mediators involved in visceral pain as bradykinin, histamine, serotonin and prostaglandin 2. Results indicated that neuronal cells' incubation with P. distasonis F1-2 significantly increased the percentage of neuronal cells inactivated at the second stimulation applied by turning off 29.77% versus 17.39% for control (FIG. 5d) (****p value<0.0001), resulting in calcium signal decreased in global kinetic (FIG. 5e).

### Example 5: P. distasonis F1-2 Neuroinhibitory Potential Involve G-Protein Coupled Receptor

Parabacteroides distasonis Culturing Methods

### Cf Example 1: Parabacteroides distasonis Culturing Methods

### ND7/23 Immortalized Neuronal Cells Culture and Treatment

ND7/23 cells were generated by fusion of cultured neonatal rat DRG neurons with N18TG2 mouse neuroblastoma cells [19]. This immortalized cell line has been characterized as a sensory neuron model [20, 21]. ND7/23 cells were cultivated in 15 ml of Dulbecco's Modified Eagle Medium (DMEM; SIGMA) supplemented with 10% (v/v) fetal bovine serum, sodium pyruvate (110 mg/mL), L-glutamine (2 mM), and penicillin (100 μg/mL), streptomycin (100 μg/mL) in 75-cm2 culture flask. At 80% confluence, cells were detached from the substrate, centrifuged at 175 g for 5 minutes and suspended in ND7/23 fresh culture medium at 800 000 cells/mL. ND7/23 suspension was plating on 96 microplate wells (Greiner bio-one 655096) at 80 000 cells/well (100 μL/well) and incubated at 37° C. overnight. Experiments on ND7/23 cell line were conducted over a period of several months and spanned on average a minimum of 30-40 passages. Number of cell passages did not significantly affect responses to bradykinin stimulation.
To test involvement of Gαi in direct interaction between P. distasonis F1-2 strain and neuronal cells, ND7/23 cells were cultivated with or without pertussis toxin (PTX, 250 ng/mL, SIGMA) and incubated at 37° C. overnight before used.

### Neuromodulatory Properties of F1-2 on ND7/23 Immortalized Neuronal Cells

After 24 h of incubation, ND7/23 cells were confluent. Culture medium was removed and replaced by 81 μL of calcium probe (Kit FLIPR Calcium 6-QF, Molecular Devices, A485 nm (excitation) and A525 nm (emission) and 81 μL of HBSS (Hank's Buffered Salt solution)-HEPES buffer 20 mM (adjusted at pH 7.4, 300 mOsm). Cells were incubated with calcium probe during 2 h at 37° C., following supplier recommendations. After incubation, calcium probe was removed and replaced by HBSS-HEPES buffer containing or not Ca²⁺.

To evaluate F1-2 effect on bradykinin EC50, ND7/23 cells were incubated with 1×10⁸ P. distasonis F1-2 viable cells/mL or vehicle (PBS+glycerol) during 45 minutes before being stimulated with bradykinin (at various final concentration). Data analysis: Maximum signal to stimulation in the second part of experiment was determined on F-FQ normalized kinetic signal. Maximum activation signal to bradykinin relative to control was represented for each bradykinin concentration tested.

### Results—Conclusions

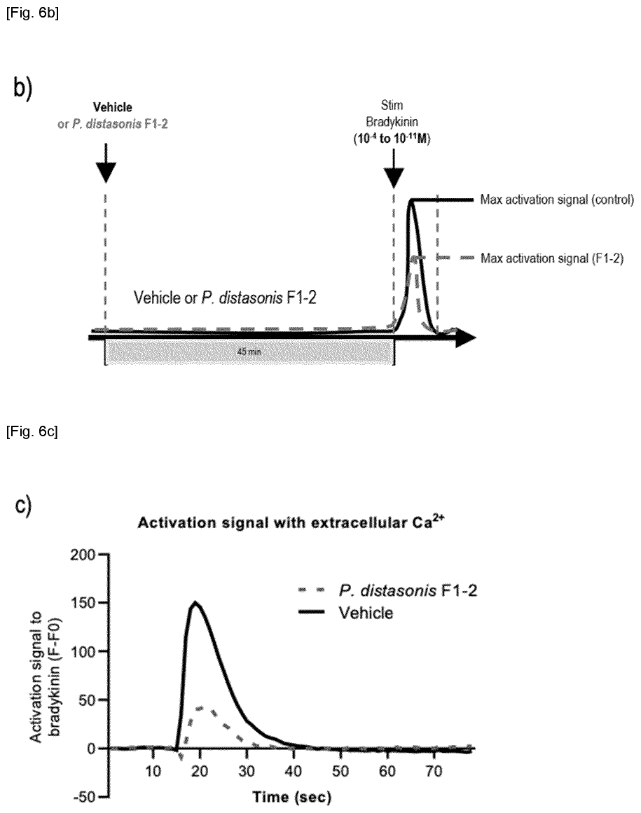

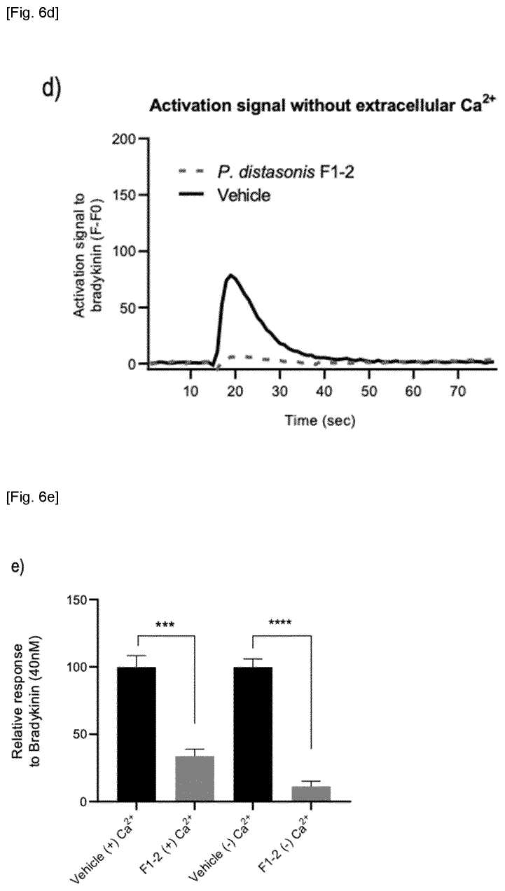

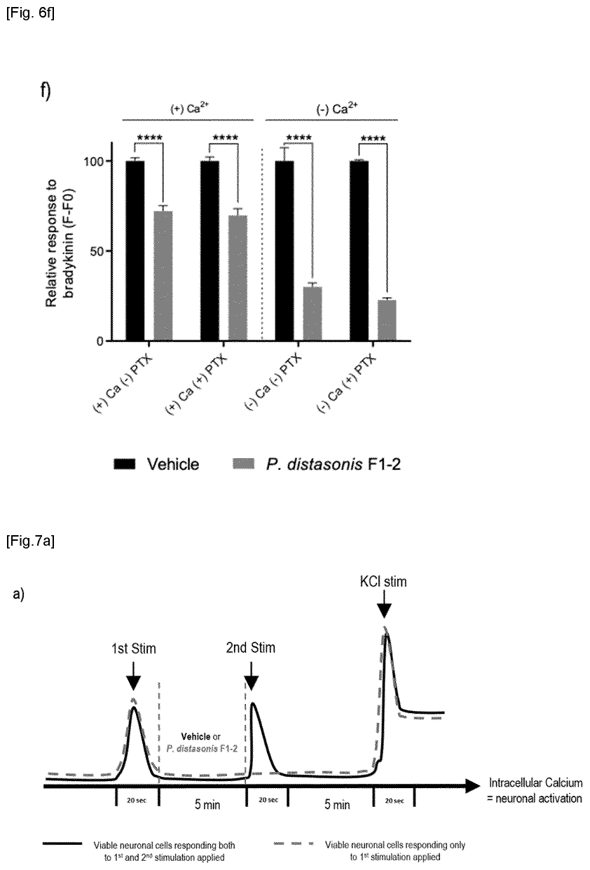

In a first experiment, immortalized ND7/23 sensory neuronal cells were incubated with P. distasonis F1-2 or vehicle during 45 min before being stimulated with various bradykinin concentrations (10 μM to 100 μM). Maximum peaks calcium signal represented on dose-response curve indicated that P. distasonis F1-2 reduced activation level of ND7/23 neuronal cells for all bradykinin concentration tested (FIG. 6a). These results indicated P. distasonis F1-2 was able to interact with bradykinin receptor by decreasing activation signal to stimulation (FIG. 6a). P. distasonis F1-2 neuro-inhibitory effect seems to be higher than those of P. distasonis ATCC 8503 reference strain (FIG. 6a).

Neuronal activation is the result of neuronal depolarization induced in part by intracellular release of calcium whose provide by endoplasmic reticulum (RE) but also by an inlet of calcium from the extracellular compartment. In fact, in our experiment, bradykinin stimulation leads to cellular pathway activation involving GPCR, leading to an increase of intracellular calcium concentration from RE. Immortalized ND7/23 sensory neuronal cells were incubated with P. distasonis F1-2 bacterial strain during 45 minutes before being stimulated with bradykinin (40 nM), with or without extracellular calcium (FIG. 6b). Deprivation of extracellular calcium allowed to measure neuronal activation specifically linked to intracellular calcium flux. Results indicated that P. distasonis F1-2 strain decreased calcium signal on ND7/23 neuronal cells (FIG. 6c and FIG. 6d). Strongest inhibitory effect was observed when neuronal cells were deprived of extracellular calcium (Vehicle (+) Ca²⁺ vs F1-2 (+) Ca²⁺: ***p value=0.0003. Vehicle (−) Ca²⁺ vs F1-2 (−) Ca²⁺: ****p<0.0001) (FIG. 6e) suggesting that G-coupled protein receptor could be involved in bacteria-neurons interaction.

Involvement of Gαi coupled protein receptor activation in neuro-inhibitory effect of P. distasonis F1-2 was evaluated by treating ND7/23 sensory neuronal cells with pertussis toxin (PTX). After 45 min of incubation with P. distasonis F1-2 strain, neuronal cells were stimulated with bradykinin (40 nM). Even if Gαi was blocked by PTX, inhibitory effect of P. distasonis F1-2 was observed (FIG. 6D with or without extracellular calcium indicating that neuro-inhibitory effect of the strain was not the result of Gαi activation.

### Example 6: P. distasonis F1-2 Neuroinhibitory Potential could Involve Bradykinin Receptor

Parabacteroides distasonis Culturing Methods

### Cf Example 1: Parabacteroides distasonis Culturing Methods

### Culture of Primary Neuronal DRG Cells

### Cf Example 4: Culture of Primary Neuronal DRG Cells

### Evaluation of Neuronal Cells Activation by Ratiometric Calcium Imaging

### Cf Example 4: Evaluation of Neuronal Cells Activation by Ratiometric Calcium Imaging

### Results—Conclusions

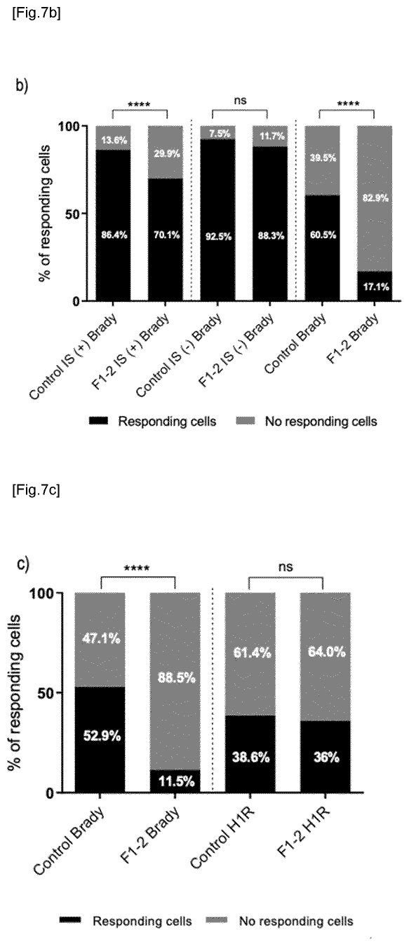

Contribution of bradykinin receptors in P. distasonis F1-2 neuroinhibitory potential was evaluated on primary neuronal cells from DRG mice. Primary neuronal cells were stimulated a first time with stimulatory solution before being incubated during 5 minutes with vehicle or with P. distasonis F1-2. Then, neuronal cells were stimulated a second time with stimulatory agent mixed with vehicle or P. distasonis F1-2 strain. A lastly KCl (50 mM) stimulation was made to control cell viability at the end of experiment (FIG. 7a). In the first experiment, stimulatory agent consisted on inflammatory soap (IS) solution targeting G-coupled protein receptors comprising bradykinin (IS (+) Brady) or not (IS (−) Brady), or bradykinin alone. Ca²⁺ responses (measured as normalized fluorescence 340 nm/380 nm ratios) of each neuronal cell was followed by ratiometric calcium imaging. Quantification of the average max ΔF/F0 of IS-positive nociceptive neurons revealed 2 respective groups in each condition. In fact, in each condition tested, 2 sub populations within responsive neurons to stimulation applied were identified. First one corresponds to responsive neurons that responded both to first and second stimulation; represented in black (FIG. 7b) whereas the second sub population corresponds to neurons that responded only to the first stimulation, represented in grey (FIG. 7b). Neuro-inhibitory potential of P. distasonis F1-2, reflected by significant increase of neuronal cells inactivated at the second stimulation, was observed only when stimulation contained bradykinin. In fact, when neuronal cells were stimulated with IS containing bradykinin, P. distasonis F1-2 cell's incubation leaded to increase inactivated neuronal cells, turning off 29.96% of responsive cells versus 13.62% for control (FIG. 7b) (****p value<0.0001). Surprisingly, inhibitory effect was lost when IS was deprived of bradykinin (FIG. 7b) while under bradykinin stimulation, neuro-inhibitory effect was observed, resulting in an increased of inactivated neuronal cells turning off 82.93% versus 39.45% for control (FIG. 7b) (****p value<0.0001).

Bradykinin receptor is a GPCR that recruiting Gαi or Gαq protein. While neuronal inhibition requires a activation in case of Gαi protein coupled receptor, inhibition of a subunit is required in case of Gαq protein coupled receptor. To evaluate interaction specificity between P. distasonis F1-2 strain and bradykinin receptor, the same experiment was conducted by replacing bradykinin stimulation by specific agonist of histamine receptor 1 or H1R (2-pyridilethylamine), which is a Gαq coupled protein receptor as bradykinin receptor. Although P. distasonis F1-2 significantly increased the percentage of no responding cells to the second bradykinin stimulation (FIG. 7c) (****p value<0.0001), neuroinhibitory effect was not observed with 2-pyridylethylamine stimulation (FIG. 6c) suggesting that H1 receptor was not involved in P. distasonis F1-2 and neuronal cells direct interaction. In addition, this result suggested that P. distasonis F1-2 does not interact specifically with Gαq protein because inhibitory effect was not observed with H1R stimulation.
Name of depositary Institution: Collection nationale de cultures de micro-organismes (CNCM)
Address of depositary Institution: Institut Pasteur, 28, rue du Dr Roux, 75724 Paris Cedex 15
Accession Number: CNCM I-5828
Date of deposit: Feb. 16, 2022

### Explanation of Sequence Listing

SEQ ID NO:1 Parabacteroides distasonis strain F1-2 according to the invention, gene for 16S ribosomal RNA.

### REFERENCES

1. Marchesi, J. R. & Ravel, J. The vocabulary of microbiome research: a proposal. Microbiome 3, 31 (2015).
2. Eckburg, P. B. et al. Diversity of the human intestinal microbial flora. Science 308, 1635-1638 (2005).
3. Sender, R., Fuchs, S. & Milo, R. Are We Really Vastly Outnumbered? Revisiting the Ratio of Bacterial to Host Cells in Humans. Cell 164, 337-340 (2016).
4. Sender, R., Fuchs, S. & Milo, R. Revised Estimates for the Number of Human and Bacteria Cells in the Body. PLOS Biology 14, e1002533 (2016).
5. Rinninella, E. et al. What is the Healthy Gut Microbiota Composition? A Changing Ecosystem across Age, Environment, Diet, and Diseases. Microorganisms 7, E14 (2019).
6. Casén, C. et al. Deviations in human gut microbiota: a novel diagnostic test for determining dysbiosis in patients with IBS or IBD. Aliment Pharmacol Ther 42, 71-83 (2015).
7. Li, J., Butcher, J., Mack, D. & Stintzi, A. Functional impacts of the intestinal microbiome in the pathogenesis of inflammatory bowel disease. Inflamm Bowel Dis 21, 139-153 (2015).
8. Lloyd-Price, J. et al. Multi-omics of the gut microbial ecosystem in inflammatory bowel diseases. Nature 569, 655-662 (2019).
9. Caenepeel, C., Sadat Seyed Tabib, N., Vieira-Silva, S. & Vermeire, S. Review article: how the intestinal microbiota may reflect disease activity and influence therapeutic outcome in inflammatory bowel disease. Aliment Pharmacol Ther 52, 1453-1468 (2020).
10. Aghazadeh, R. et al. Inflammatory bowel disease in Iran: a review of 457 cases. J Gastroenterol Hepatol 20, 1691-1695 (2005).
11. Ceuleers, H. et al. Visceral hypersensitivity in inflammatory bowel diseases and irritable bowel syndrome: The role of proteases. World J Gastroenterol 22, 10275-10286 (2016).
12. Jones, R. C. W., Xu, L. & Gebhart, G. F. The mechanosensitivity of mouse colon afferent fibers and their sensitization by inflammatory mediators require transient receptor potential vanilloid 1 and acid-sensing ion channel 3. J Neurosci 25, 10981-10989 (2005).
13. Akbar, A. et al. Expression of the TRPV1 receptor differs in quiescent inflammatory bowel disease with or without abdominal pain. Gut 59, 767-774 (2010).
14. Akbar, A. et al. Increased capsaicin receptor TRPV1-expressing sensory fibres in irritable bowel syndrome and their correlation with abdominal pain. Gut 57, 923-929 (2008).
15. Miranda, A. et al. The role of transient receptor potential vanilloid 1 in mechanical and chemical visceral hyperalgesia following experimental colitis. Neuroscience 148, 1021-1032 (2007).
16. Gottesman-Katz, L., Latorre, R., Vanner, S., Schmidt, B. L. & Bunnett, N. W. Targeting G protein-coupled receptors for the treatment of chronic pain in the digestive system. Gut 70, 970-981 (2021).
17. Sakamoto, M. & Benno, Y. Reclassification of Bacteroides distasonis, Bacteroides goldsteinii and Bacteroides merdae as Parabacteroides distasonis gen. nov., comb. nov., Parabacteroides goldsteinii comb. nov. and Parabacteroides merdae comb. nov. International Journal of Systematic and Evolutionary Microbiology 56, 1599-1605 (2006).
18. Hiippala, K. et al. Isolation of Anti-Inflammatory and Epithelium Reinforcing Bacteroides and Parabacteroides Spp. from A Healthy Fecal Donor. Nutrients 12, 935 (2020).
19. Wood, J. N. et al. Novel cell lines display properties of nociceptive sensory neurons. Proceedings of the Royal Society of London. Series B: Biological Sciences 241, 187-194 (1990).
20. Haberberger, R. V., Barry, C. & Matusica, D. Immortalized Dorsal Root Ganglion Neuron Cell Lines. Front. Cell. Neurosci. 14, (2020).
21. Kobrinsky, E. M., Pearson, H. A. & Dolphin, A. C. Low- and high-voltage-activated calcium channel currents and their modulation in the dorsal root ganglion cell line ND7-23. Neuroscience 58, 539-552 (1994).

1. A Parabacteroides distasonis strain deposited under accession number CNCM I-5828.

2. The Parabacteroides distasonis strain according to claim 1, wherein the 16s rRNA gene sequence of the Parabacteroides distasonis strain is as shown in SEQ ID NO:1.

3. The Parabacteroides distasonis strain according to any one of claims 1 and 2, wherein said strain is isolated from healthy human feces.

4. A nutritional supplement composition comprising a bacterial strain according to any one of the preceding claims.

5. The nutritional supplement composition according to the preceding claim, further comprising at least one acceptable nutritional ingredient, preferably chosen among: probiotics, prebiotics, symbiotics, parabiotics, metabiotics vitamins, minerals, herbs, amino acids and enzymes.

6. A composition comprising a bacterial strain according to any one of claims 1 to 3, for use as a medicament.

7. The composition for use according to the preceding claim, for use in treating and/or preventing visceral pain.

8. The composition for use according to the preceding claim, wherein said visceral pain is induced by Inflammatory Bowel Disease including Crohn's disease and Ulcerative Colitis, or by Irritable Bowel Syndrome.

9. The nutritional supplement composition according to any one of claims 4 to 5, or the composition for use according to any one of claims 6 to 8, wherein the composition comprises at least 10⁹, preferably at least 10¹⁰, colony forming units/mL of the Parabacteroides distasonis strain deposited under accession number CNCM I-5828.

10. The nutritional supplement composition according to any one of claims 4, 5 and 9, or the composition for use according to any one of claims 6 to 9, wherein the composition only comprises living bacteria.

11. The nutritional supplement composition according to any one of claims 4, 5 and 9, or the composition for use according to any one of claims 6 to 9, wherein the composition only comprises killed bacteria and/or fractions of bacteria.

12. The composition for use according to any one of claims 6 to 11, further comprising at least one other acceptable active pharmaceutical ingredient, and/or at least one acceptable pharmaceutical excipient and/or at least one acceptable pharmaceutical carrier.

13. The composition for use according to any one of claims 6 to 12, wherein the composition is for oral, intravenous, in intestine or subcutaneous administration, preferably oral administration.

14. The composition for use according to the preceding claim, wherein the oral administration is made by gelatin capsules, capsules, tablets, powders, granules, oral solutions or suspensions.

15. A cell of the Parabacteroides distasonis strain deposited under accession number CNCM I-5828
